(19) United States
(12) Patent Application Publication
(10) Pub. No.: US 2017/0121391 A1
(43) Pub. Date: May 4, 2017

(54) POTENT GLYCOPROTEIN ANTIBODY AS A THERAPEUTIC AGAINST EBOLA VIRUS

(71) Applicant: Wisconsin Alumni Research Foundation (WARF), Madison, WI (US)

(72) Inventors: Yoshihiro Kawaoka, Middleton, WI (US); Peter J. Halfmann, Madison, WI (US); Ayato Takada, Toyohira-ku (JP)

(21) Appl. No.: 15/204,381

(22) Filed: Jul. 7, 2016

Related U.S. Application Data
(60) Provisional application No. 62/189,466, filed on Jul. 7, 2015.

(51) Int. Cl. C07K 16/10

(52) U.S. Cl. CPC C07K 16/10; C07K 2317/76

(57) ABSTRACT

Antibodies useful to prevent, inhibit or treat Ebola virus infection, vectors encoding and host cells expressing one or more heavy chains or light chains that bind Ebola virus, are provided.

### CROSS-REFERENCE TO RELATED APPLICATIONS

This application claims the benefit of the filing date of U.S. application Ser. No. 62/189,466, filed on Jul. 7, 2015, the disclosure of which is incorporated by reference herein.

### STATEMENT OF GOVERNMENT RIGHTS

This invention was made with government support under AI057153 awarded by the National Institutes of Health. The government has certain rights in the invention.

### BACKGROUND

Ebola virus, a filamentous, enveloped, nonsegmented negative-strand RNA virus in the family Filoviridae, has caused sporadic outbreaks of lethal hemorrhagic disease for which no effective vaccine or antiviral treatment is available. The virus contains at least seven structural proteins, all of which are translated from monocistronic polyadenylated mRNA transcripts (Feldmann and Kiley, 1999; Sanchez et al., 2001). The fourth gene from the 3′ end of the Ebola virus genome encodes two glycoproteins: the envelope glycoprotein (GP), which is responsible for receptor binding and fusion of the virus with the host cell membrane (Takada et al., 1997; Wool-Lewis and Bates, 1998), and the nonstructural secretory glycoprotein (sGP), which is released from infected cells (Sanchez et al., 1996; Volchkov et al., 1995).

Since GP is the only viral surface protein responsible for virus entry (Takada et al., 1997; Wool-Lewis and Bates, 1998), it must be an important target of neutralizing antibodies. However, DNA immunization of mice with the GP of the Zaire species of Ebola virus produced infectivity-enhancing antibodies, as well as neutralizing antibodies, raising issues about the development of passive prophylaxis or treatment with Ebola virus GP antibodies (Takada, 2001). The passive transfer of hyperimmune animal sera has been evaluated in mice, guinea pigs, and monkeys (Jahrling et al., 1999; Jahrling et al., 1996; Kudoyarova-Zubavichene et al., 1999; Peters and Khan, 1999) with inconsistent results. Although whole-blood transfusion from convalescent patients was also tested in patients during the Kikwit outbreak of Ebola hemorrhagic fever in 1995 (Mupapa, 1999), reliable conclusions could not be drawn from these studies owing to the inevitable lack of controls. Serum from mice subcutaneously infected with live Ebola virus protected recipient mice from a lethal challenge (Gupta et al., 2001). However, it is unclear whether virus-induced immune factors other than antibodies (e.g., cytokines) may have affected the efficacy of such treatment. Although the protective efficacy of immune sera varies, as described above, passive transfer of neutralizing MAbs completely protected mice from a lethal Ebola virus infection (Wilson et al., 2000).

B-cell epitopes are not well defined on Ebola virus GP. Thus, it is important not only to analyze the antigenic structure of the proteins hut also to understand the mechanisms by which the antibodies interfere with the protein's function (e.g., inhibition of viral receptor binding and fusion). By using synthetic peptides derived from amino acid sequences of Ebola virus species Zaire GP, Wilson et al. (2000) identified three epitopes recognized by neutralizing antibodies. However, it is generally believed that the use of synthetic peptides provides limited information about the B-cell epitopes of heavily glycosylated proteins such as Ebola virus GP (Sanchez et al., 2001). Since sugar chains are often important in the tertiary structure of these proteins, small synthetic peptides are not usually identical to those of the corresponding regions in the actual glycoprotein. Finally, synthetic peptides do not provide an optimal means of identifying conformational epitopes.

One potential therapeutic option in the treatment of Ebola virus infection is antibody therapy. ZMapp, a combination of three monoclonal antibodies, is one such therapy in human clinical trials.

### SUMMARY

The nucleotide sequences disclosed herein, when recombinantly expressed, have therapeutic activity against Ebola virus. In one embodiment, the expressed polypeptides have therapeutic activity against Ebola virus that is equal to or greater than the ZMapp cocktail. A side-by-side blinded comparison of over 85 Ebola virus glycoprotein monoclonal antibodies including those antibodies in the ZMapp cocktail, were screened for activity. In particular, mAb226, described herein, demonstrated potent therapeutic activity against Ebola virus. In one embodiment, a disclosed polypeptide, e.g., in the form of an antibody, that is encoded by an expression vector, may be administered before or after exposure to Ebola virus, e.g., 1, 2, 3, 4, 5 or more days after exposure. In one embodiment, neutralizing antibody cocktails (including antibodies such as those disclosed herein) provide for cross-reactive protection. In one embodiment, an expression vector (e.g., DNA or RNA vector) encoding a polypeptide having one or more of the disclosed antibody variable region sequences, e.g., encoding a ScFv, is administered to a mammal. In one embodiment, the expression vector encodes an antibody heavy chain comprising a variable region having SEQ ID NO:1 or SEQ ID NO:3, or a polypeptide with at least 80%, 85%, 90%, 95%, 97%, 98% or more amino acid sequence identity thereto. In one embodiment, the expression vector encodes an antibody light chain comprising a variable region having SEQ ID NO:2 or SEQ ID NO:4 or a polypeptide with at least 80%, 85%, 90%, 95%, 97%, 98% or more amino acid sequence identity thereto. In one embodiment, a sequence with less than 100% identity to one of SEQ ID NOs. 1-4 is one with one or more substitutions relative to SEQ ID NOs:1-4, including conservative substitutions. In one embodiment, a sequence with less than 100% identity to one of SEQ ID NOs. 1-4 is one with one or more substitutions relative to SEQ ID NOs:1-4, including non-conservative substitutions. In one embodiment, a sequence with less than 100% identity to one of SEQ ID NOs. 1-4 is one with one or more substitutions relative to SEQ ID NOs:1-4, including a combination of conservative and non-conservative substitutions. In one embodiment, the invention relates to antibody sequences, as described herein, including any functional parts (fragment or portion) thereof, which antibody or part thereof binds to Ebola virus and may have 1, 2, 3, 4, 5 or at least 10, and up to 20 or 30 substitutions, e.g., conservative substitutions, relative to a polypeptide having one of SEQ ID Nos. 1-4. In one embodiment, the expression vector comprises a nucleotide sequence with at least 80%, 85%, 90%, 95%, 97%, 98% or more nucleic acid sequence identity to a nucleic acid sequence encoding an antibody heavy chain comprising a variable region having SEQ ID NO:1 or SEQ ID NO:3, or a polypeptide with at least 80%, 85%, 90%, 95%, 97%, 98% or more amino acid sequence identity thereto, wherein the nucleotide sequence encodes a heavy chain that alone or in combination with a light chain, binds Ebola virus with substantially the same efficiency as, e.g., a Kd(M) within about 0.5, 1 or 2 logs that of or has a protective effect that is at least 70% that of, an antibody heavy chain comprising a variable region having SEQ ID NO:1 or SEQ ID NO:3 alone or in combination with a light chain. In one embodiment, the expression vector comprises a nucleotide sequence with at least 80%, 85%, 90%, 95%, 97%, 98% or more nucleic acid sequence identity to a nucleic acid sequence encoding an antibody light chain comprising a variable region having SEQ ID NO:2 or SEQ ID NO:4, or a polypeptide with at least 80%, 85%, 90%, 95%, 97%, 98% or more amino acid sequence identity thereto, wherein the nucleotide sequence encodes a light chain that alone or in combination with a heavy chain, binds Ebola virus with substantially the same efficiency, e.g., a Kd(M) within about 0.5, 1 or 2 logs that of or has a protective effective that is at least 70% that of, an antibody light chain comprising a variable region having SEQ ID NO:2 or SEQ ID NO:4 alone or in combination with a heavy chain. In one embodiment, the nucleotide sequence includes one or more nucleotide substitutions, relative to the nucleic acid sequence encoding one of SEQ ID NO:1-4, that increase expression of the antibody sequences in a host cell.

In one embodiment of the invention, nucleic acid sequences, or host cells, e.g., CHO cells, having at least one of the disclosed variable regions, encoding an antibody or a polypeptide having SEQ ID NO:1 or SEQ ID NO:3, or a polypeptide with at least 80%, 85%, 90%, 95%, 97%, 98% or more amino acid sequence identity thereto, SEQ ID NO:2 or SEQ ID NO:4 or a polypeptide with at least 80%, 85%, 90%, 95%, 97%, 98% or more amino acid sequence identity thereto, are provided, e.g., a polypeptide, such as an Ig heavy chain, Ig light chain, or a fusion of Ig heavy and light chain sequences, including any functional parts thereof, which bind to, neutralizes or otherwise inhibits Ebola virus infection or replication.

Accordingly, it is an embodiment to provide host cells expressing an antibody, e.g., a monoclonal antibody, including any functional parts thereof as described herein, which antibody is capable of inhibiting Ebola virus infection in a mammal.

Accordingly, it is an embodiment to provide a method of making and using an antibody, including any functional parts thereof, which antibody or part thereof is capable of inhibiting Ebola virus infection or replication in a mammal. In one embodiment, the antibody according to the present invention, including any functional parts thereof, binds to the Zaire strain of Ebola virus. An antibody according to the invention including any functional parts thereof may decrease viral load by at least 20%, by at least 25%, by at least 30%, or more than 30%.

Thus, a vector is provided having a nucleic acid sequence encoding a polypeptide having SEQ ID NO:1, a polypeptide having SEQ ID NO:2 or a polypeptide having at least 90% amino acid identity to SEQ ID NO:1 or 2, or an Ebola virus antigen binding fragment of the polypeptide. In one embodiment, the nucleic acid sequence is a cDNA, In one embodiment, the nucleic acid sequence is not SEQ ID NO:5. In one embodiment, the nucleic acid sequence is not SEQ ID NO:2& In one embodiment, the polypeptide having SEQ ID NO:1 is encoded by any of SEQ ID NO:5-25. In one embodiment, the polypeptide having SEQ ID NO:2 is encoded by any of SEQ ID NO:26-46. In one embodiment, the vector further comprises a promoter In one embodiment, the polypeptide has at least 95% amino acid identity to SEQ ID NO:1 or 2. In one embodiment, the polypeptide encodes an Ig heavy chain or an Ig light chain, or a ScFv. In one embodiment, the vector encodes an IgG or IgA heavy chain. The vector may be employed as a nucleic acid vaccine.

Also provided is a vector having a nucleic acid sequence encoding a polypeptide having SEQ ID NO:3, a polypeptide having SEQ ID NO:4, or a polypeptide having at least 90% amino acid identity to SEQ ID NO:3 or 4, or an Ebola virus binding fragment of the polypeptide. In one embodiment, the polypeptide having SEQ ID NO:3 is encoded by any of SEQ ID NO:48-68, In one embodiment, the nucleic acid sequence is a cDNA. In one embodiment, the nucleic acid sequence is not SEQ ID NO:48. In one embodiment, the nucleic acid sequence is not SEQ ID NO:69. In one embodiment, the polypeptide having SEQ ID NO:4 is encoded by any of SEQ ID NO:69-89. In one embodiment, the vector further comprises a promoter. In one embodiment, the polypeptide has at least 95% amino acid identity to SEQ ID NO:3 or 4. In one embodiment, the polypeptide encodes an Ig heavy chain or an Ig light chain, or a ScFv. In one embodiment, the vector encodes an IgG or IgA heavy chain. The vector may be employed as a nucleic acid vaccine.

Further provided is an isolated host cell having one or more of the vectors. In one embodiment, the host cell is a bacterium. In one embodiment, the host cell is a mammalian cell. In one embodiment, the host cell is not a hybridoma. In one embodiment, the host cell is an insect cell. In one embodiment, the host cell is a plant cell, e.g., a dicot cell. Host cells useful to express antibody sequences include but are not limited to mammalian cells, e.g., CHO cells, PERC.6 cells and HEK293 cells, yeast cells, bacterial cells, insect cells, and plant cells, e.g., tobacco cells. Further provide is a hybridoma which secretes the antibodies described herein. For example, a hybridoma is provided that secretes an antibody comprising a polypeptide comprising at least 90% amino acid sequence identity to SEQ ID NO:1, and optionally comprising a polypeptide comprising at least 90% amino acid sequence identity to SEQ ID NO:2; or an isolated antibody comprising a polypeptide comprising at least 90% amino acid sequence identity to SEQ ID NO:2, and optionally comprising a polypeptide comprising at least 90% amino acid sequence identity to SEQ ID NO:1; or an isolated antibody comprising a polypeptide comprising at least 90% amino acid sequence identity to SEQ ID NO:3, and optionally comprising a polypeptide comprising at least 90% amino acid sequence identity to SEQ ID NO:4; or an isolated antibody comprising a polypeptide comprising at least 90% amino acid sequence identity to SEQ ID NO:4, and optionally comprising a polypeptide comprising at least 90% amino acid sequence identity to SEQ ID NO:3.

Also provided is a composition comprising a combination of antibodies including an antibody or an Ebola virus binding portion thereof comprising, or a polypeptide having SEQ ID NO:1, 2, 3 or 4, or a sequence with at least 90% amino acid sequence identity thereto. In one embodiment, the composition comprises a combination of antibodies including an antibody comprising a polypeptide having SEQ ID NO:1, 2, 3 or 4, or a sequence with at least 90% amino acid sequence identity thereto, or an Ebola virus binding portion thereof.

The vectors, polypeptides, antibodies and parts thereof, and compositions having the vectors, polypeptide or antibody or antibody parts, including a composition having a combination of antibodies, may be employed in a method to prevent, inhibit or treat Ebola virus infection. In one embodiment, an effective amount of a composition having an antibody comprising a polypeptide comprising SEQ ID NO:1 or a polypeptide having at least 90% amino acid identity thereto, or an Ebola virus binding portion thereof, and a polypeptide comprising SEQ ID NO:1 or a polypeptide having at least 90% amino acid identity thereto, or an Ebola virus binding portion thereof, is administered. In one embodiment, an effective amount of a composition having an antibody comprising a polypeptide comprising SEQ ID NO:3 or a polypeptide having at least 90% amino acid identity thereto, or an Ebola virus binding portion thereof, and a polypeptide comprising SEQ ID NO:4 or a polypeptide having at least 90% amino acid identity thereto, or an Ebola virus binding portion thereof, is administered. Also provided is an isolated antibody comprising a polypeptide having at least 90% but not 100% identity to SEQ ID NO:1, comprising a polypeptide having at least 90% but not 100% identity to SEQ ID NO:2, comprising a polypeptide having at least 90% but not 100% identity to SEQ ID NO:3, or comprising a polypeptide having at least 90% but not 100% identity to SEQ ID NO:4.

In one embodiment, an antibody comprising a polypeptide having at least 90% but not 100% identity to SEQ ID NO:1 and comprising a polypeptide having at least 90% identity to SEQ ID NO:2 is provided. In one embodiment, an antibody comprising a polypeptide having at least 90% identity to SEQ ID NO:1 and comprising a polypeptide having at least 90% identity but not 100% to SEQ ID NO:2 is provided. In one embodiment, an antibody comprising a polypeptide having at least 90% but not 100% identity to SEQ ID NO:1 and comprising a polypeptide having at least 90% identity but not 100% to SEQ ID NO:2 is provided. In one embodiment, the antibody comprises IgG1.

In one embodiment, an antibody comprising a polypeptide having at least 90% but not 100% identity to SEQ ID NO:3 and comprising a polypeptide having at least 90% identity to SEQ ID NO:4 is provided. In one embodiment, an antibody comprising a polypeptide having at least 90% identity to SEQ ID NO:3 and comprising a polypeptide having at least 90% identity but not 100% to SEQ ID NO:4 is provided. In one embodiment, an antibody comprising a polypeptide having at least 90% but not 100% identity to SEQ ID NO:3 and comprising a polypeptide having at least 90% identity but not 100% to SEQ ID NO:4 is provided. In one embodiment, the antibody comprises IgG1.

### BRIEF DESCRIPTION OF THE FIGURES

FIGS. 1A-1E. Heavy chain variable region sequences for MAb 133/316 (SEQ ID NOs:5-25 (nucleic acid)).

FIGS. 2A-2E. Light chain variable region sequences for MAb 133/316 (SEQ ID NOs:26-46 (nucleic acid)).

FIGS. 3A-3D. Heavy chain variable region sequences for MAb 226/81 (SEQ ID NOs:48-68 (nucleic acid)).

FIGS. 4A-4D, Light chain variable region sequences for MAb 226/81 (SEQ ID NOs:69-89 (nucleic acid)).

FIGS. 5A-5FF. Representative genome sequences for the Zaire strain of Ebola virus (SEQ ID NOs:90-93).

FIG. 6A-6B. Heavy chain and light chain variable region amino acid and nucleotide sequences for MAb 226/81 (SEQ ID NO:3 and 4 (amino acid) and SEQ ID NOs:7 and 8 (murine nucleic acid)), and heavy chain and light chain variable region amino acid and nucleotide sequences for MAb 133/316 (SEQ ID NO:1 and 2 (amino acid) and SEQ ID NOs:5 and 6 (murine nucleic acid)).

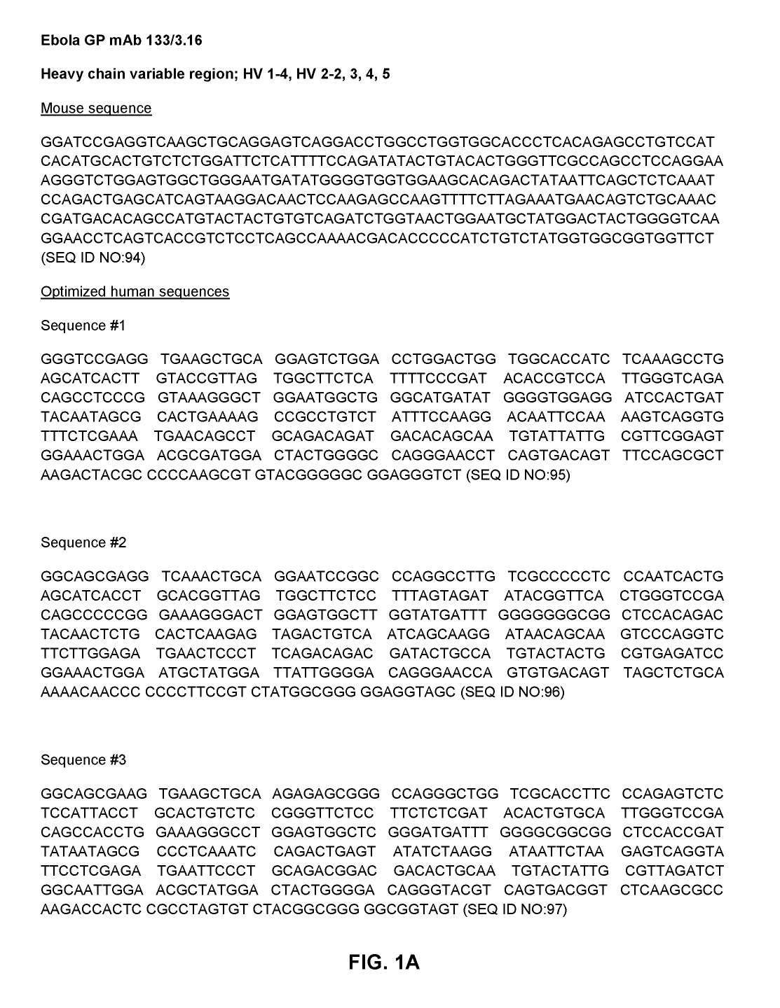

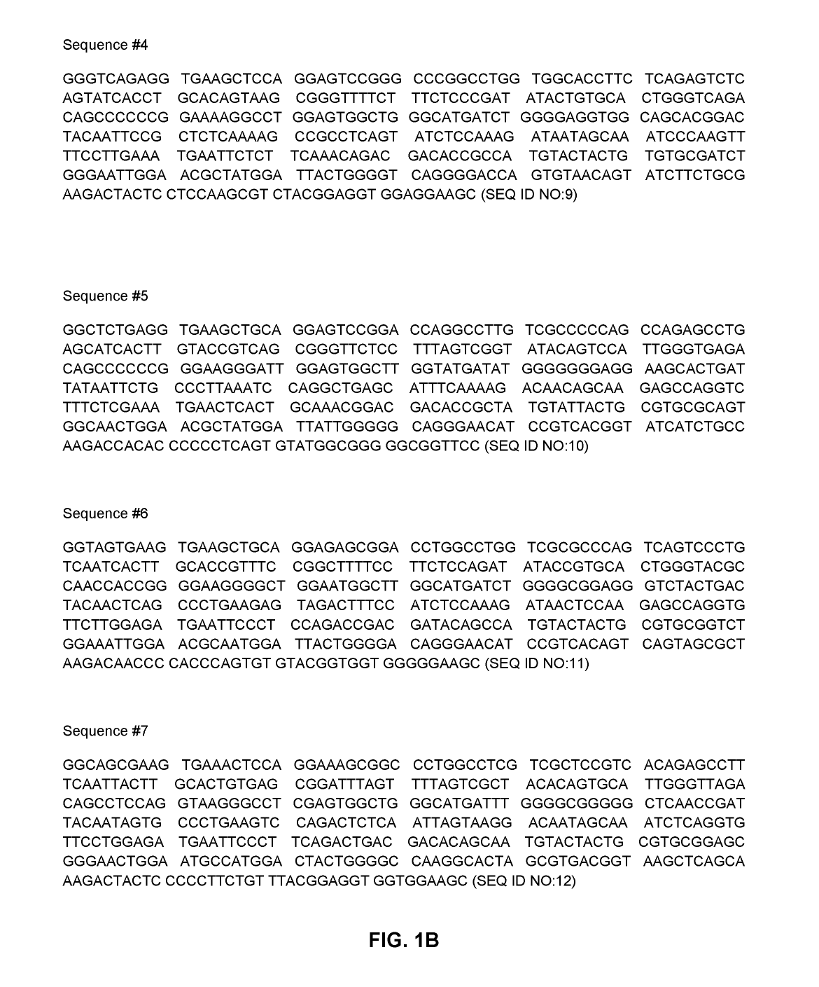

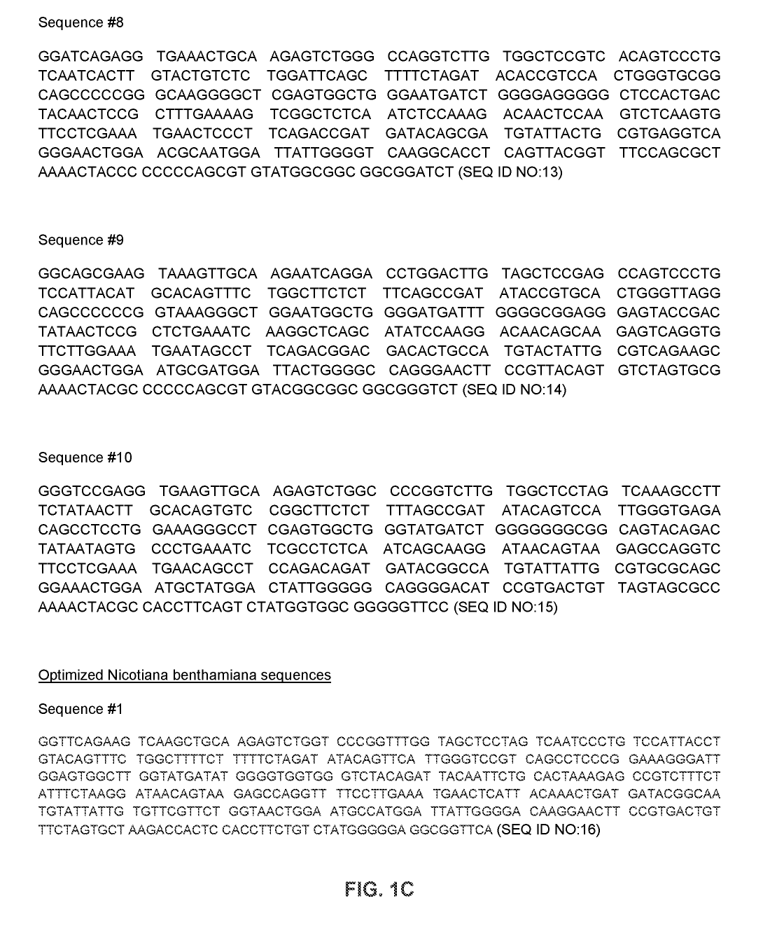

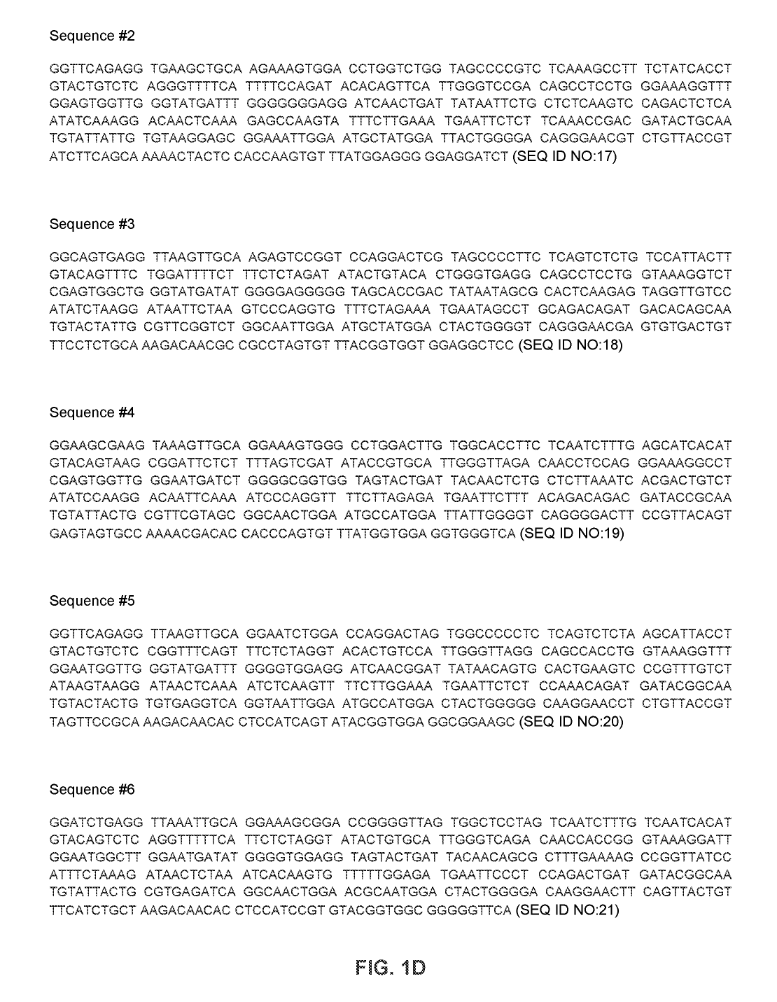

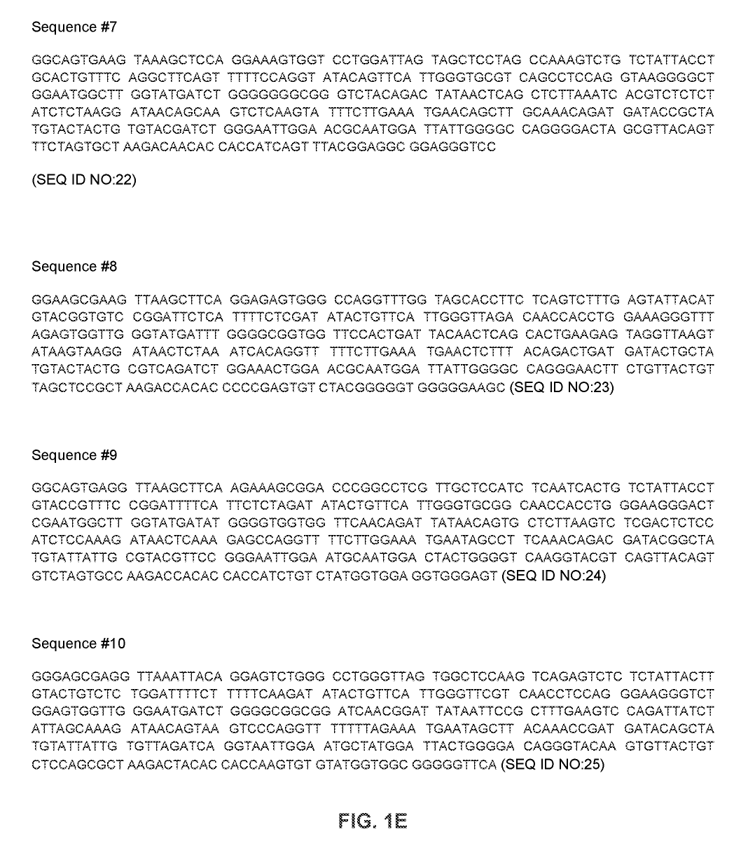

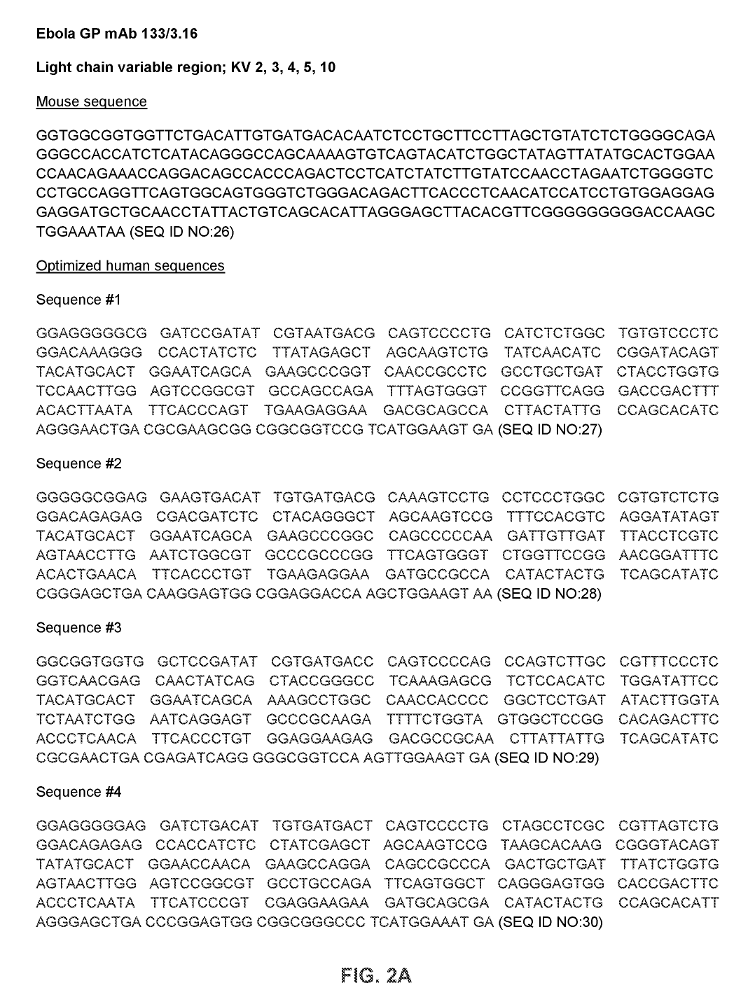

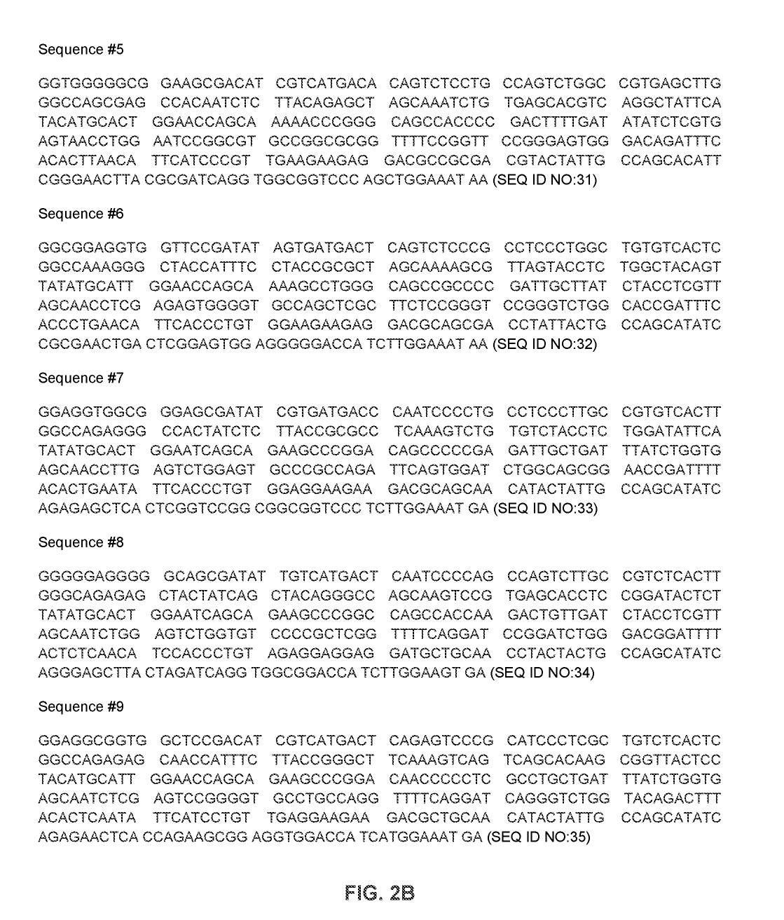

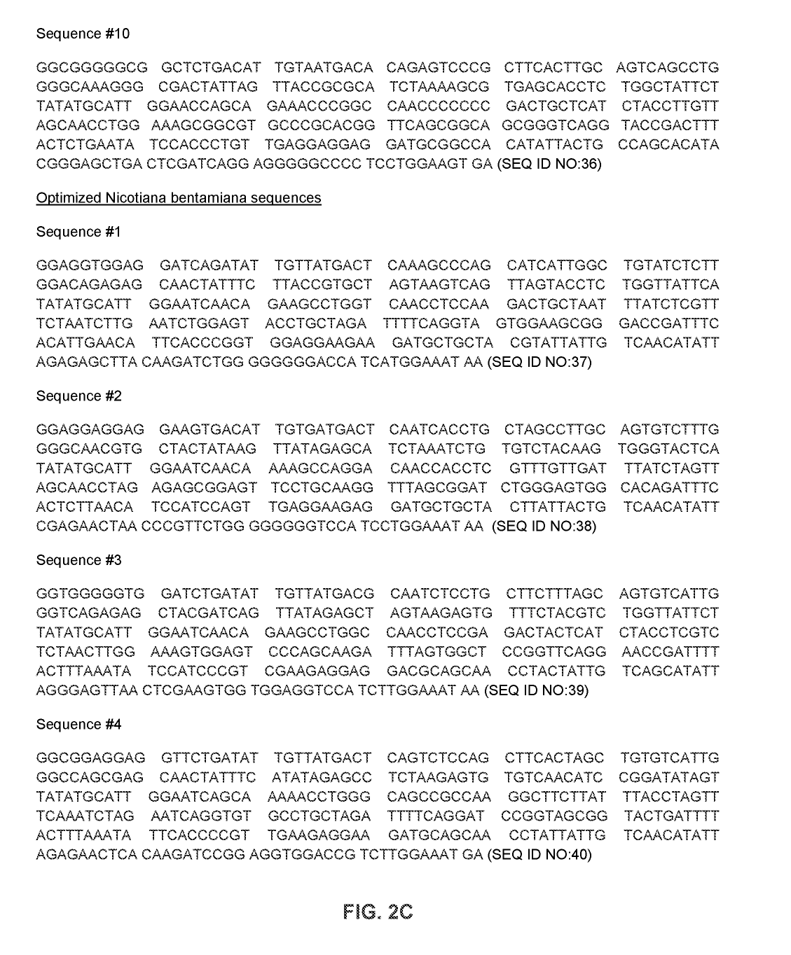

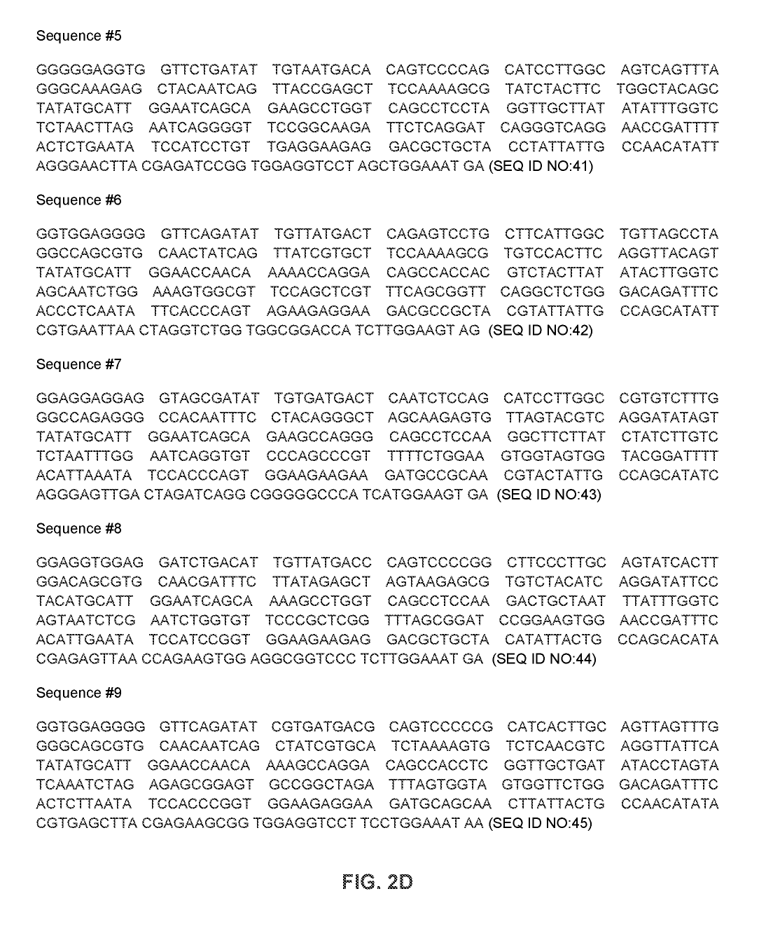

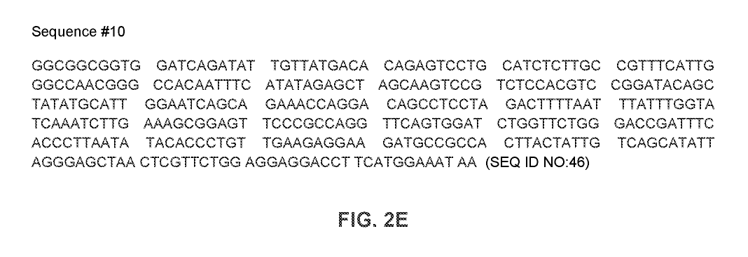

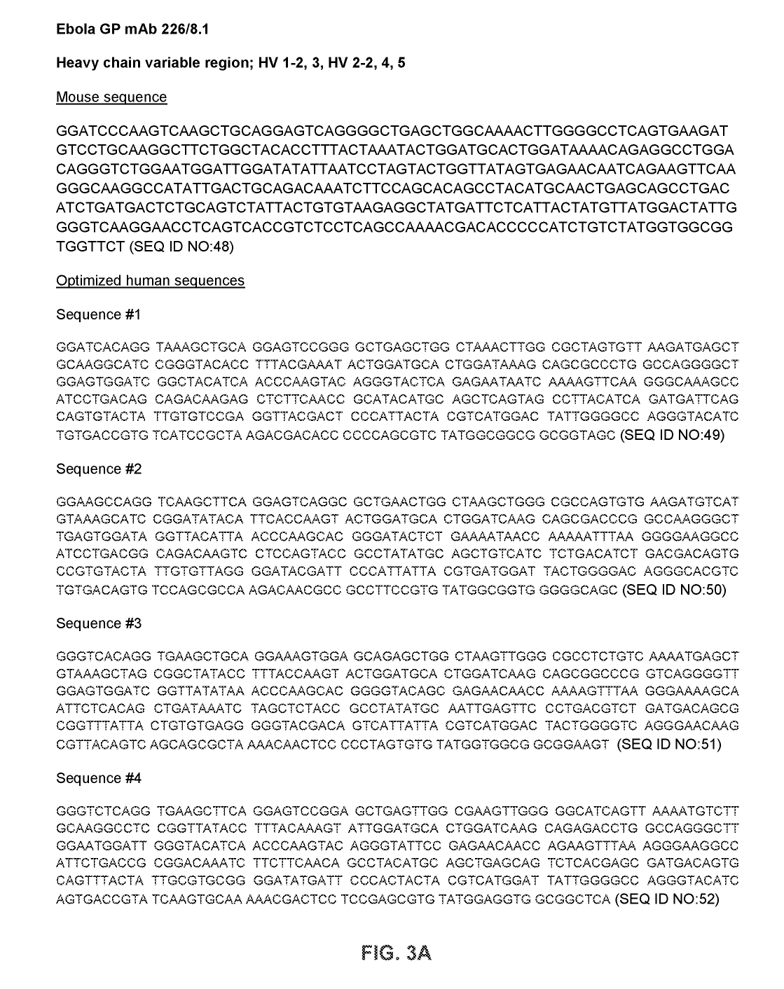

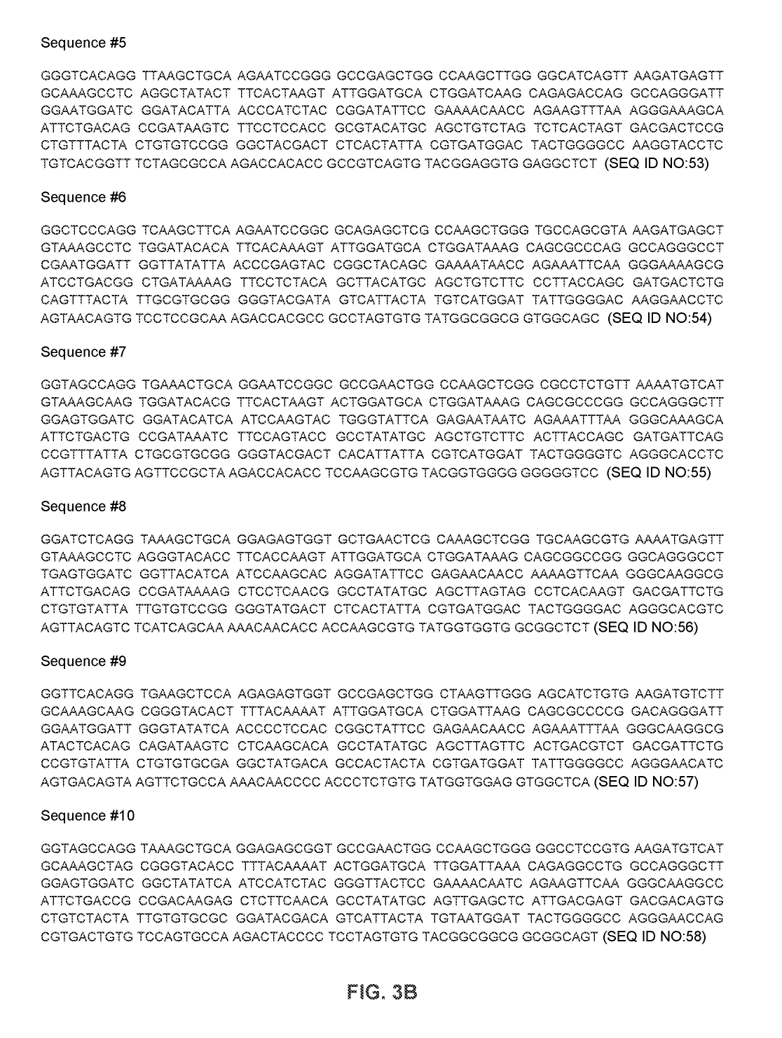

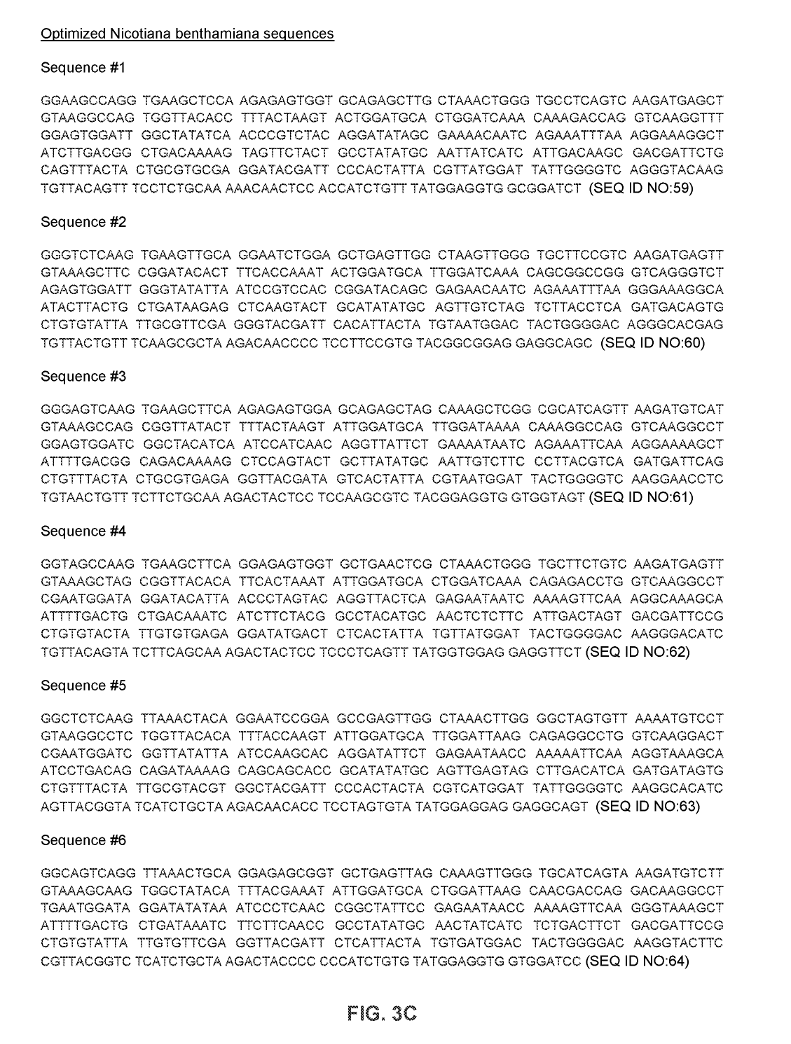

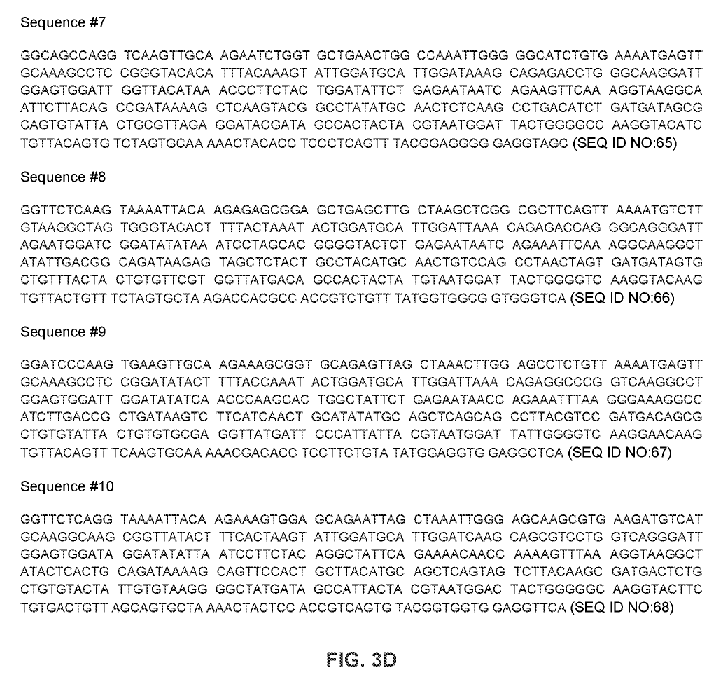

### DETAILED DESCRIPTION

### Definitions

As used herein with respect to polypeptides, the term “substantially pure” means that the polypeptides are essentially free of other substances with which they may be found in nature or in vivo systems to an extent practical and appropriate for their intended use. In particular, the polypeptides are sufficiently pure and are sufficiently free from other biological constituents of their host cells so as to be useful in, for example, generating antibodies, sequencing, or producing pharmaceutical preparations. By techniques well known in the art, substantially pure polypeptides may be produced in light of the nucleic acid and amino acid sequences disclosed herein. Because a substantially purified polypeptide of the invention may be admixed with a pharmaceutically acceptable carrier in a pharmaceutical preparation, the polypeptide may comprise only a certain percentage by weight of the preparation. The polypeptide is nonetheless substantially pure in that it has been substantially separated from the substances with which it may be associated in living systems.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

As used herein, a coding sequence and regulatory sequences are said to be “operably joined” or “operably linked” when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribing and 5′ non-translating sequences involved with initiation of transcription and translation respectively, such as a TATA box, capping sequence, CAAT sequence, and the like. Especially, such 5′ non-transcribing regulatory sequences will include a promoter region which includes a promoter sequence for transcriptional control of the operably joined gene. Regulatory sequences may also include enhancer sequences or upstream activator sequences, as desired.

As used herein, a “vector” may be any of a number of nucleic acids into which a desired sequence may be inserted by restriction and ligation for transport between different genetic environments or for expression in a host cell, Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell, An expression TO vector is one into which a desired DNA sequence may be inserted by restriction and ligation such that it is operably joined to regulatory sequences and may be expressed as an RNA transcript. Vectors may further contain one or more marker sequences suitable for use in the identification and selection of cells which have been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. In some embodiments, the vectors are those capable of autonomous replication and expression of the structural gene products present in the DNA segments to which they are operably joined.

The terms “polypeptide”, “peptide”, and “protein”, as used herein, are interchangeable and are defined to mean a biomolecule composed of amino acids linked by a peptide bond.

The terms “a”, “an” and “the” as used herein are defined to mean “one or more” and include the plural unless the context is inappropriate.

The terms “detecting” or “detected” as used herein mean using known techniques for detection of biologic molecules such as immunochemical or histological methods and refer to qualitatively or quantitatively determining the presence or concentration of the biomolecule under investigation. For example, the binding of an antibody including any functional parts thereof, to Ebola virus may be determined by an ELISA-type or immunofluorence-based assay.

The terms “antibody” or “antibodies” as used herein are art recognized terms and are understood to refer to molecules or active fragments of molecules that bind to known antigens, particularly to immunoglobulin molecules and to immunologically active portions of immunoglobulin molecules, i.e. molecules that contain a binding site that immunospecifically binds an antigen. The immunoglobulin according to the invention can be of any type (IgG, IgM, IgD, IgE, IgA and IgY) or class (IgG1, IgG2, IgG3, IgG4, IgA1 and IgA2) or subclasses of immunoglobulin molecule.

“Antibodies” are intended within the scope of the present invention to include monoclonal, polyclonal, chimeric, single chain, bispecific or bi-effective, simianized, human and humanized antibodies as well as active fragments thereof. Examples of active fragments of molecules that bind to known antigens include Fab, F(ab′)₂, scFv and Fv fragments, including the products of an Fab immunoglobulin expression library and epitope-binding fragments of any of the antibodies and fragments mentioned above, Such active fragments can be derived from an antibody of the present invention by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw et al., (1982); Rousseaux et al., (1986).

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al. (1988). Antibodies or immunoglobulins include broad classes of polypeptides that can be distinguished biochemically. Those skilled in the art will appreciate that heavy chains are classified as gamma, mu, alpha, delta, or epsilon, with some subclasses among them (e.g., gamma1-gamma4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernible to the skilled artisan in view of the instant disclosure and JCV neutralizing antibodies of different classes can be obtained or engineered as described herein. It should be appreciated that all immunoglobulin classes are within the scope of the present invention. However, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda. Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As described herein, the variable region allows the antibody to selectively recognize and specifically bind epitopes on antigens. That is, the VL domain and VH domain, or subset of the complementarity determining regions (CDRs), of an antibody combine to form the variable region that defines a three dimensional antigen binding site. This quaternary antibody structure forms the antigen binding site present at the end of each arm of the Y, More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al. (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a beta-sheet conformation and the CDRs form loops which connect, and in some cases form part of, the beta-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, Kabat et al. (1983); and Chothia and Leak, (1987)), which are incorporated herein by reference in their entireties).

It should be appreciated that antibodies obtained as described herein can be altered to remove or replace one or more CDRs. In some embodiments, antigen binding fragments can be generated that retain antigen specificity but that lack one or more of the six CDRs of a full-length antibody. Alternatively, one or more CDRs from an antibody can be retained (for example CDR3) and one or more of the other CDRs can be engineered and or replaced with a different CDR, for example, to alter antigen binding specificity and/or affinity.

As used herein, the term “heavy chain portion” includes amino acid sequences derived from an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. For example, a binding polypeptide for use in the invention may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the invention comprises a polypeptide chain comprising a CH3 domain. Further, a binding polypeptide for use in the invention may lack at least a portion of a CH2 domain (e.g., all or part of a CH2 domain). As set forth above, it will be understood by one of ordinary skill in the art that these domains (e.g., the heavy chain portions) may be modified such that they vary in amino acid sequence from the naturally occurring immunoglobulin molecule.

As used herein, the term “light chain portion” includes amino acid sequences derived from an immunoglobulin light chain. In one embodiment, the light chain portion comprises at least one of a VL or CL domain.

By “specifically binds,” it is generally meant that an antibody binds to an epitope via its antigen binding domain, and that the binding entails some complementarity between the antigen binding domain and the epitope. An antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope.

An antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

The minimum size of a peptide or polypeptide epitope for an antibody is thought to be about four to five amino acids. Peptide or polypeptide epitopes may contain at least seven, at least nine or between at least about 15 to about 30 amino acids. Since a CDR can recognize an antigenic peptide or polypeptide in its tertiary form, the amino acids comprising an epitope need not be contiguous, and in some cases, may not even be on the same peptide chain. In some embodiments, a peptide or polypeptide epitope recognized by neutralizing antibodies may contain a sequence of at least 4, at least 5, at least 6, at least 7, e.g., at least 8, at least 9, at least 10, at least 15, at least 20, at least 25, or about 15 to about 30 contiguous or non-contiguous amino acids.

As used herein, the term “affinity” refers to a measure of the strength of the binding of an individual epitope with the CDR of an immunoglobulin molecule. See, e.g., Harlow et al. (1988). As used herein, the term “avidity” refers to the overall stability of the complex between a population of immunoglobulins and an antigen, that is, the functional combining strength of an immunoglobulin mixture with the antigen. See, e.g., Harlow, Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Neutralizing antibodies, or antigen-binding fragments, variants, or derivatives thereof disclosed herein may be described or specified in terms of the epitope(s) or portion(s) of an antigen, e.g., a target polypeptide of the Ebola virus protein that they recognize or specifically bind. The portion of a target polypeptide which specifically interacts with the antigen binding domain of an antibody is an “epitope,” or an “antigenic determinant.” A target polypeptide may comprise a single epitope, but typically comprises at least two epitopes, and can include any number of epitopes, depending on the size, conformation, and type of antigen, Furthermore, it should be noted that an “epitope” on a target polypeptide may be or include non-polypeptide elements, e.g., an “epitope may include a carbohydrate side chain.

Neutralizing antibodies or antigen-binding fragments, variants or derivatives thereof described herein may, also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

Neutralizing antibodies or antigen-binding fragments, variants or derivatives thereof described herein may also be described or specified in terms of their binding affinity to a polypeptide. For example, a Ebola virus neutralizing antibody may bind to a Ebola virus peptide with a dissociation constant or Kd less than 10⁻²M, 10⁻³M, 10⁻⁴M, 10⁻⁵M, 10⁻⁸M, 10⁻⁹M, 10⁻¹⁰M, 10⁻¹¹M, 10⁻¹²M, 10⁻¹³M, 10⁻¹⁴M, or 10⁻¹⁵M.

Neutralizing antibodies or antigen-binding fragments, variants or derivatives thereof described herein may be “multispecific,” e.g., bispecific, trispecific or of greater multispecificity, meaning that it recognizes and binds to two or more different epitopes present on one or more different antigens (e.g., proteins) at the same time. Thus, whether a neutralizing antibody is “monospecfic” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity, Methods to obtain “humanized antibodies” are well known to those skilled in the art. (See, e.g., Queen et al., (1989), Hodgson et al., (1991)).

A “humanized antibody” may also be obtained by a novel genetic engineering approach that enables production of affinity-matured humanlike polyclonal antibodies in large animals such as, for example, rabbits (see, e.g. U.S. Pat. No. 7,129,084).

The term “monoclonal antibody” is also well recognized in the art and refers to an antibody that is mass produced in the laboratory from a single clone and that recognizes only one antigen. Monoclonal antibodies are typically made by fusing a normally short-lived, antibody-producing B cell to a fast-growing cell, such as a cancer cell (sometimes referred to as an “immortal” cell), The resulting hybrid cell, or hybridoma, multiplies rapidly, creating a clone that produces large quantities of the antibody. For the purpose of the present invention, “monoclonal antibody” is also to be understood to comprise antibodies that are produced by a mother clone which has not yet reached full monoclonality.

The term “CDR” refers to the hypervariable region of an antibody. The term “hypervariable region”, “HVR”, or “HV”, when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions are based on sequence variability and are the most commonly used (Kabat et al., Sequences of Proteins of Immunological Interest, 5th Ed. Public Health Service, National Institutes of Health, Bethesda, Md. (1991)).

The letters “HC” and “LC” preceding the term “CDR” refer, respectively, to a CDR of a heavy chain and a light chain. Chothia refers instead to the location of the structural loops (Chothia and Leak J. Mol. Biol. 196:901-917 (1987)). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these hypervariable regions are noted below.

| Loop | Kabat | AbM | Chothia | Contact |
|---|---|---|---|---|
| L1 | L24-L34 | L24-L34 | L26-L32 | L30-L36 |
| L2 | L50-L58 | L50-L56 | L50-L52 | L46-L55 |
| L3 | L89-L97 | L89-L97 | L91-L96 | L89-L96 |
| H1 | H31-H35B | H28-H35B | H28-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

Hypervariable regions may comprise “extended hypervariable regions” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., Sequences of Proteins of immunological interest, 5th Ed. Public Health Service, National Institutes of Health, Bethesda, Md. (1991).

Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

In camelid species, the heavy chain variable region, referred to as VHH, forms the entire antigen-binding domain. The main differences between camelid VHH variable regions and those derived from conventional antibodies (VH) include (a) more hydrophobic amino acids in the light chain contact surface of VH as compared to the corresponding region in VHH, (b) a longer CDR3 in VHH, and (c) the frequent occurrence of a disulfide bond between CDR1 and CDR3 in VHH.

“Functional part” is understood within the scope of the present invention to refer to a polypeptide or complex of polypeptides which substantially shares at least one major functional property with an antibody, for example, binding to or inhibiting infection or replication by, for example, the Zaire strain of Ebola virus, when administered prophylactically or therapeutically. The antibodies can be of any class such as IgG, IgM, or IgA, etc or any subclass such as IgG1, IgG2a, etc and other subclasses described herein above or known in the art, but particularly of the IgG4 class. Further, the antibodies can be produced by any method, such as phage display, or produced in any organism or cell line, including bacteria, insect, mammal or other type of cell or cell line which produces antibodies with desired characteristics, such as humanized antibodies. Antibodies can also be formed by combining a Fab portion and an Fc region from different species.

The term “bispecific” or “bifunctional” and “hi-effective” is used synonymously within the scope of this application to characterize an antibody which exhibits both an inhibition property on amyloid or amyloid-like fiber formation as well as a disaggregation property of amyloid or amyloid-like fibers.

As used herein, the term “soluble” means partially or completely dissolved in an aqueous solution.

Also as used herein, the term “immunogenic” refers to substances which elicit or enhance the production of antibodies, T-cells or other reactive immune cells directed against an immunogenic agent, e.g., Ebola virus, and contribute to an immune response in humans or animals.

The term “hybridoma” is art recognized and is understood by those of ordinary skill in the art to refer to a cell produced by the fusion of an antibody-producing cell and an immortal cell, e.g., a multiple myeloma cell. Such a hybrid cell is capable of producing a continuous supply of antibody, See the definition of “monoclonal antibody” above and the Examples below for a more detailed description of one art known method of fusion.

Further, the term “therapeutically effective amount” refers to the amount of antibody or polypeptide, or DNA encoding the polypeptide or antibody, which, when administered to a human or animal, elicits an immune response which is sufficient to result in a therapeutic effect in said human or animal. The effective amount is readily determined by one of ordinary skill in the art following routine procedures.

“Homology” between two sequences is determined by sequence identity. If two sequences which are to be compared with each other differ in length, sequence identity may relate to the percentage of the nucleotide residues of the shorter sequence which are identical with the nucleotide residues of the longer sequence, Sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711), Bestfit utilizes the local homology algorithm of Smith and Waterman, (1981), in order to find the segment having the highest sequence identity between two sequences. When using Bestfit or another sequence alignment program to determine whether a particular sequence has for example 95% identity with a reference sequence of the present invention, the parameters may be adjusted so that the percentage of identity is calculated over the entire length of the reference sequence and homology gaps of up to 5% of the total number of the nucleotides in the reference sequence are permitted. When using Bestfit, the so-called optional parameters may be left at their preset (“default”) values. The deviations appearing in the comparison between a given sequence and the above-described sequences of the invention may be caused for instance by addition, deletion, substitution, insertion or recombination. Such a sequence comparison may also be carried out with the program “fasta20u66” (version 2.0u66, September 1998 by William R. Pearson and the University of Virginia; see also Pearson (1990), appended examples and http://workbench.sdsc.edu/). For this purpose, the “default” parameter settings may be used.

As used herein a “conservative change” refers to alterations that are substantially conformationally or antigenically neutral, producing minimal changes in the tertiary structure of the mutant polypeptides, or producing minimal changes in the antigenic determinants of the mutant polypeptides, respectively, as compared to the native protein. When referring to the antibodies and antibody fragments of the invention, a conservative change means an amino acid substitution that does not render the antibody incapable of binding to the subject receptor. One of ordinary skill in the art will be able to predict which amino acid substitutions can be made while maintaining a high probability of being conformationally and antigenically neutral, Such guidance is provided, for example in Berzofsky (1985) and Bowie et al, (1990), Factors to be considered that affect the probability of maintaining conformational and antigenic neutrality include, but are not limited to: (a) substitution of hydrophobic amino acids is less likely to affect antigenicity because hydrophobic residues are more likely to be located in a protein's interior; (b) substitution of physiochemically similar, amino acids is less likely to affect conformation because the substituted amino acid structurally mimics the native amino acid; and (c) alteration of evolutionarily conserved sequences is likely to adversely affect conformation as such conservation suggests that the amino acid sequences may have functional importance. One of ordinary skill in the art will be able to assess alterations in protein conformation using well-known assays, such as, but not limited to microcomplement fixation methods (see, e.g. Wasserman et al. (1961); Levine et al. (1967)) and through binding studies using conformation-dependent monoclonal antibodies (see, e.g. Lewis et al. (1983)).

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine: a group of amino acids having amide-containing side chains is asparagine and glutamine; a group of amino acids having aromatic side chains is phenylalanine, tyrosine and tryptophan; a group of amino acids having basic side chains is lysine, arginine and histidine: and a group of amino acids having sulfur-containing side chain is cysteine and methionine. In one embodiment, conservative amino acid substitution groups are: threonine-valine-leucine-isoleucine-alanine; phenylalanine-tyrosine; lysine-arginine; alanine-valine; glutamic-aspartic; and asparagine-glutamine.

The term “hybridize” as used herein refers to conventional hybridization conditions, e.g., hybridization conditions at which 5×SSPE, 1% SDS, 1×Denhardts solution is used as a solution and/or hybridization temperatures are between 35° C. and 70° C., for instance, 65° C. After hybridization, washing may be carried out first with 2×SSC, 1% SDS and subsequently with 0.2×SSC at temperatures between 35° C. and 70° C., e.g., at 65° C. (regarding the definition of SSPE, SSC and Denhardts solution see Sambrook et al. Molecular Biology: A Laboratory Manual, Cold Spring Harbor Press, Cold Spring Harbor, N.Y., 1989). Stringent hybridization conditions as for instance described in Sambrook et al, supra, may be employed. In one embodiment, stringent hybridization conditions are for instance present if hybridization and washing occur at 65° C. as indicated above. Non-stringent hybridization conditions, for instance with hybridization and washing carried out at 45° C. or at 35° C. or even less.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of an antibody or other material other than directly into the central nervous system, such that it enters the patient's system and, thus, is subject to metabolism and other like processes, for example, subcutaneous administration.

### Antibody Sequences

The present invention derives, in part, from the isolation and characterization of antibodies that selectively bind to and/or neutralize Ebola virus and are defined by the amino acid (aa) sequences of the immunoglobulin heavy and light chain V-regions described in SEQ ID NO:1 through SEQ ID NO:4.

In one set of embodiments, the present invention provides full-length antibodies or fragments thereof in isolated form and in pharmaceutical preparations. Similarly, as described below, the present invention provides isolated nucleic acids, host cells transformed with nucleic acids, and pharmaceutical preparations including isolated nucleic acids encoding the antibodies or fragments thereof, isolated polypeptides or isolated antibodies or parts thereof. The present invention also provides methods, as described more fully below, employing these antibodies and nucleic acids in the in vitro and in vivo diagnosis, prevention and therapy of Ebola virus infection.

Significantly, as is well-known in the art, only a small portion of an antibody molecule is involved in the binding of the antibody to its epitope (see, in general, Clark (1986); Roitt, (1991)). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of a full-length antibody. Similarly, an antibody from which the Fc region has been enzymatically cleaved, or which has been produced without the Fc region, designated an Fab fragment, retains one of the antigen binding sites of a full-length antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general. Clark, 1986, supra; Roitt, 1991, supra). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

The non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of full-length antibodies with antigen-binding ability, are often referred to as “chimeric” antibodies.

Thus, as will be apparent to one of ordinary skill in the art, the present invention also provides for F(ab′)2, Fab, Fv and Fd fragments of anti-Ebola virus antibodies; chimeric antibodies in which the Fc and/or FR and/or CDR1 and/or CDR2 and/or light chain CDR3 regions of the anti-Ebola virus antibodies have been replaced by homologous human or non-human sequences; chimeric F(ab′)2 fragment antibodies in which the FR and/or CDR1 and/or CDR2 and/or light chain CDR3 regions of the anti-Ebola virus antibodies have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. Thus, those skilled in the art may alter the anti-Ebola virus antibodies by the construction of CDR grafted or chimeric antibodies or antibody fragments containing all, or part thereof, of the disclosed heavy and light chain V-region CDR aa sequences (Jones et al, (1986); Verhoeyen et al. (1988); and Tempest et al. (1991)), without destroying the specificity of the antibodies. Such CDR grafted or chimeric antibodies or antibody fragments can be effective in prevention and treatment of Ebola virus infection in animals (e.g., horses) and man.

In some embodiments, the antibodies are fully human monoclonal antibodies including at least the heavy chain CDR3 region of the Ebola virus antibodies. As noted above, such chimeric antibodies may be produced in which some or all of the FR regions of the anti-Ebola virus antibodies have been replaced by other homologous human FR regions. In addition, the Fc portions may be replaced so as to produce IgA or IgM as well as IgG antibodies bearing some or all of the CDRs of the anti-Ebola virus antibodies, Of particular importance is the inclusion of the Ebola virus antibodies heavy chain CDR3 region and, to a lesser extent, the other CDRs of the anti-Ebola virus antibodies. Such fully human or chimeric antibodies will have particular utility in that they will not evoke an immune response against the antibody itself.

For inoculation or prophylactic uses, the antibodies of the present invention may be full-length antibody molecules including the Fc region. Such full-length antibodies often have longer half-lives than smaller fragment antibodies (e.g., Fab) and may be more suitable for intravenous, intraperitoneal, intramuscular, intracavity, subcutaneous, or transdermal administration.

In some embodiments, Fab fragments, including chimeric Fab fragments, may be employed. Fabs offer several advantages over F(ab′)₂ and whole immunoglobulin molecules for this therapeutic modality. First, because Fabs have only one binding site for their cognate antigen, the formation of immune complexes is precluded whereas such complexes can be generated when bivalent F(ab′)₂ s and whole immunoglobulin molecules encounter their target antigen. This is of some importance because immune complex deposition in tissues can produce adverse inflammatory reactions. Second, because Fabs lack an Fc region they cannot trigger adverse inflammatory reactions that are activated by Fc, such as activation of the complement cascade. Third, the tissue penetration of the small Fab molecule is likely to be much better than that of the larger whole antibody. Fourth. Fabs can be produced easily and inexpensively in bacteria, such as E. coli, whereas whole immunoglobulin antibody molecules require mammalian cells for their production in useful amounts. The latter entails transfection of immunoglobulin sequences into mammalian cells with resultant transformation, Amplification of these sequences must then be achieved by rigorous selective procedures and stable transformants must be identified and maintained. The whole immunoglobulin molecules must be produced by stably transformed, high expression mammalian cells in culture with the attendant problems of serum-containing culture medium. In contrast, production of Fabs in E. coli eliminates these difficulties and makes it possible to produce these antibody fragments in large fermenters which are less expensive than cell culture-derived products.

In addition to Fabs, smaller antibody fragments and epitope-binding peptides having binding specificity for the epitopes defined by the anti-Ebola virus antibodies are also contemplated by the present invention and can also be used to bind or neutralize the virus. For example, single chain antibodies can be constructed according to the method of U.S. Pat. No. 4,946,778, to Ladner et al. Single chain antibodies comprise the variable regions of the light and heavy chains joined by a flexible linker moiety. Yet smaller is the antibody fragment known as the single domain antibody or Fd, which comprises an isolated VH single domain. Techniques for obtaining a single domain antibody with at least some of the binding specificity of the full-length antibody from which they are derived are known in the art.

It is possible to determine whether an altered or chimeric antibody or fragment thereof has the same specificity as the anti-Ebola virus antibodies by ascertaining whether the former blocks the latter from binding to the virus. If the antibody or fragment thereof being tested competes with the anti-Ebola virus antibody as shown by a decrease in binding of the anti-Ebola virus antibody, then it is likely that the two monoclonal antibodies bind to the same, or a closely spaced, epitope. Still another way to determine whether an antibody has the specificity of the anti-Ebola virus antibodies is to pre-incubate the anti-Ebola virus antibody with the virus with which it is normally reactive, and then add the antibody being tested to determine if the antibody being tested is inhibited in its ability to bind the virus. If the antibody being tested is inhibited then, in all likelihood, it has the same, or a functionally equivalent, epitope and specificity as the disclosed anti-Ebola virus antibodies. Screening of anti-Ebola virus antibodies also can be carried out by utilizing Ebola viruses and determining whether the mAb neutralizes the virus.

By using the disclosed antibodies, it is now possible to produce anti-idiotypic antibodies which can be used to screen other antibodies to identify whether the antibody has the same binding specificity as an antibody of the invention. In addition, such antiidiotypic antibodies can be used for active immunization (Herlyn et al. (1986)). Such anti-idiotypic antibodies can be produced using well-known hybridoma techniques (Kohler and Milstein (1975)). An anti-idiotypic antibody is an antibody which recognizes unique determinants present on the monoclonal antibody produced by the cell line of interest. These determinants are located in the hypervariable region of the antibody. It is this region which binds to a given epitope and, thus, is responsible for the specificity of the antibody. An anti-idiotypic antibody can be prepared by immunizing an animal with the monoclonal antibody of interest. The immunized animal will recognize and respond to the idiotypic determinants of the immunizing antibody and produce an antibody to these idiotypic determinants. By using the anti-idiotypic antibodies of the immunized animal, it is possible to identify other clones with the same idiotype as the antibody of the hybridoma used for immunization. Idiotypic identity between monoclonal antibodies of two cell lines demonstrates that the two monoclonal antibodies are the same with respect to their recognition of the same epitopic determinant. Thus, by using anti-idiotypic antibodies, it is possible to identify other hybridomas expressing monoclonal antibodies having the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the image of the epitope bound by the first monoclonal antibody. Thus, the anti-idiotypic monoclonal antibody can be used for immunization, since the anti-idiotype monoclonal antibody binding domain effectively acts as an antigen.

### Nucleic Acids Encoding Anti-Ebola Virus Antibodies

Given the disclosure herein of the amino acid sequences of the heavy chain Fd and light chain variable domains of the anti-Ebola virus antibodies, one of ordinary skill in the art is now enabled to produce nucleic acids which encode this antibody or which encode the various fragment antibodies or chimeric antibodies described above. It is contemplated that such nucleic acids will be operably joined to other nucleic acids forming a recombinant vector for cloning or for expression of the antibodies. The present invention includes any recombinant vector containing the coding sequences, or part thereof, whether for prokaryotic or eukaryotic transformation, transfection or gene therapy. Such vectors may be prepared using conventional molecular biology techniques, known to those with skill in the art, and would comprise DNA coding sequences for the immunoglobulin V-regions of the anti-Ebola virus antibodies, including framework and CDRs or parts thereof, and a suitable promoter either with (Whittle et al. (1987) and Burton et al, (1994)) or without (Marasco et al. (1993) and Duan et al. (1994)) a signal sequence for export or secretion. Such vectors may be transformed or transfected into prokaryotic (Huse et al. (1989); Ward et al. (1989); Marks et al. (1991); and Barbas et al. (1991)) or eukaryotic (Whittle et al. (1987) and Burton et al. (1994)) cells or used for gene therapy (Marasco et al. (1993) and Duan et al. (1994)) by conventional techniques, known to those with skill in the art.

The expression vectors of the present invention include regulatory sequences operably joined to a nucleotide sequence encoding one of the antibodies. As used herein, the term “regulatory sequences” means nucleotide sequences which are necessary for or conducive to the transcription of a nucleotide sequence which encodes a desired polypeptide and/or which are necessary for or conducive to the translation of the resulting transcript into the desired polypeptide. Regulatory sequences include, but are not limited to, 5′ sequences such as operators, promoters and ribosome binding sequences, and 3′ sequences such as polyadenylation signals. The vectors of the invention may optionally include 5′ leader or signal sequences, 5′ or 3′ sequences encoding fusion products to aid in protein purification, and various markers which aid in the identification or selection of transformants. The choice and design of an appropriate vector is within the ability and discretion of one of ordinary skill in the art. The subsequent purification of the antibodies may be accomplished by any of a variety of standard means known in the art.

One vector for screening antibodies, but not necessarily for the mass production of antibodies, is a recombinant DNA molecule containing a nucleotide sequence that codes for and is capable of expressing a fusion polypeptide containing, in the direction of amino- to carboxy-terminus, (1) a secretion signal domain, (2) a polypeptide of the invention, and, optionally, (3) a fusion protein domain. The vector includes DNA regulatory sequences for expressing the fusion polypeptide, in one embodiment, eukaryotic, regulatory sequences, Such vectors can be constructed by those with skill in the art and have been described by Smith et al. (1985); Clackson et al. (1991); Kang et al. (1991); Barbas et al. (1991); Roberts et al. (1992)).

To achieve high levels of gene expression in E. Golf, it is necessary to use not only strong promoters to generate large quantities of mRNA, but also ribosome binding sites to ensure that the mRNA is efficiently translated. In E. coli, the ribosome binding site includes an initiation codon (AUG) and a sequence 3-9 nucleotides long located 3-11 nucleotides upstream from the initiation codon (Shine and Dalgarno (1975)). The sequence, which is called the Shine-Dalgarno (SD) sequence, is complementary to the 3′ end of E, coil 16S rRNA. Binding of the ribosome to mRNA and the sequence at the 3′ end of the mRNA can be affected by several factors: the degree of complementarity between the SD sequence and 3′ end of the 16S rRNA; the spacing lying between the SD sequence and the AUG; and the nucleotide sequence following the AUG, which affects ribosome binding. The 3′ regulatory sequences define at least one termination (stop) codon in frame with and operably joined to the heterologous fusion polypeptide.

In some embodiments with a prokaryotic expression host, the vector utilized includes a prokaryotic origin of replication or replicon, i.e., a DNA sequence having the ability to direct autonomous replication and maintenance of the recombinant DNA molecule extrachromosomally in a prokaryotic host cell, such as a bacterial host cell, transformed therewith. Such origins of replication are well known in the art. In one embodiment, the origins of replication are those that are efficient in the host organism. One host cell is E. coli. For use of a vector in E. coli, one origin of replication is ColEI found in pBR322 and a variety of other common plasmids. Also, the p15A origin of replication found on pACYC and its derivatives. The ColEI and p15A replicons have been extensively utilized in molecular biology, are available on a variety of plasmids and are described by Sambrook et al., 1989, in Molecular Cloning: A Laboratory Manual, 2nd edition, Cold Spring Harbor Laboratory Press.

In addition, those embodiments that include a prokaryotic replicon may also include a gene whose expression confers a selective advantage, such as drug resistance, to a bacterial host transformed therewith. Typical bacterial drug resistance genes are those that confer resistance to ampicillin, tetracycline, neomycin/kanamycin or chloramphenicol. Vectors typically also contain convenient restriction sites for insertion of translatable DNA sequences. Exemplary vectors are the plasmids pUC18 and pUC19 and derived vectors such as those commercially available from suppliers such as Invitrogen (San Diego, Calif.).

When the antibodies include both heavy chain and light chain sequences, these sequences may be encoded on separate vectors or, more conveniently, may be expressed by a single vector. The heavy and light chain may, after translation or after secretion, form the heterodimeric structure of natural antibody molecules. Such a heterodimeric antibody may or may not be stabilized by disulfide bonds between the heavy and light chains.

A vector for expression of heterodimeric antibodies, such as the full-length antibodies or the F(ab)₂, Fab or Fv fragment antibodies, is a recombinant DNA molecule adapted for receiving and expressing translatable first and second DNA sequences, That is, a DNA expression vector for expressing a heterodimeric antibody provides a system for independently cloning (inserting) the two translatable DNA sequences into two separate cassettes present in the vector, to form two separate cistrons for expressing the first and second polypeptides of a heterodimeric antibody. The DNA expression vector for expressing two cistrons is referred to as a dicistronic expression vector.

The vector comprising a first cassette includes upstream and downstream DNA regulatory sequences operably joined via a sequence of nucleotides adapted for directional ligation to an insert DNA. The upstream translatable sequence may encode the secretion signal. The cassette includes DNA regulatory sequences for expressing the first antibody polypeptide that is produced when an insert translatable DNA sequence (insert DNA) is directionally inserted into the cassette via the sequence of nucleotides adapted for directional ligation.

A dicistronic expression vector also contains a second cassette for expressing the second antibody polypeptide. The second cassette includes a second translatable DNA sequence that may encode a secretion signal, as described above, operably joined at its 3′ terminus via a sequence of nucleotides adapted for directional ligation to a downstream DNA sequence of the vector that typically defines at least one stop codon in the reading frame of the cassette. The second translatable DNA sequence is operably joined at its 5′ terminus to DNA regulatory sequences forming the 5′ elements. The second cassette is capable, upon insertion of a translatable DNA sequence (insert DNA), of expressing the second fusion polypeptide comprising a secretion signal with a polypeptide coded by the insert DNA.

The antibodies of the present invention may be produced by eukaryotic cells such as CHO cells, insect cells, human or mouse hybridomas, immortalized B-lymphoblastoid cells, and the like. In this case, a vector is constructed in which eukaryotic regulatory sequences are operably joined to the nucleotide sequences encoding the antibody polypeptide or polypeptides. The design and selection of an appropriate eukaryotic vector is within the ability and discretion of one of ordinary skill in the art. The subsequent purification of the antibodies may be accomplished by any of a variety of standard means known in the art.

The antibodies of the present invention may furthermore, of course, be produced in plants. In 1989, Hiatt et al. (1989) first demonstrated that functional antibodies could be produced in transgenic plants. Since then, a considerable amount of effort has been invested in developing plants for antibody (or “plantibody”) production (for reviews see Giddings et al. (2000); Fischer and Emans, (2000)). Recombinant antibodies can be targeted to seeds, tubers, or fruits, making administration of antibodies in such plant tissues advantageous for immunization programs in developing countries and worldwide.

In another embodiment, the present invention provides host cells, both prokaryotic and eukaryotic, transformed or transfected with, and therefore including, the vectors of the present invention.

### Diagnostic and Pharmaceutical Antibody Preparations

The invention also relates to a method for preparing diagnostic or pharmaceutical compositions comprising antibodies or polynucleotide sequences encoding the disclosed antibodies or parts thereof, the pharmaceutical compositions being used for immunoprophylaxis or immunotherapy of Ebola virus. The pharmaceutical preparation includes a pharmaceutically acceptable carrier. Such carriers, as used herein, means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The antibodies are suited for in vitro use, for example, in immunoassays in which they can be utilized in liquid phase or bound to a solid phase carrier. In addition, the monoclonal antibodies in these immunoassays can be detectably labeled in various ways, Examples of types of immunoassays which can utilize the antibodies are competitive and non-competitive immunoassays in either a direct or indirect format. Examples of such immunoassays are the radioimmunoassay (RIA) and the sandwich (immunometric) assay. Detection of antigens using the disclosed antibodies can be done utilizing immunoassays which are run in either the forward, reverse, or simultaneous modes, including immunohistochemical assays on physiological samples. Those of skill in the art will know, or can readily discern, other immunoassay formats without undue experimentation.

The I antibodies can be bound to many different carriers and used to detect the presence of Ebola virus. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble for purposes of the invention. Those skilled in the art will know of other suitable carriers for binding monoclonal antibodies, or will be able to ascertain such, using routine experimentation.

For purposes of the invention. Ebola virus may be detected by antibodies when present in biological fluids and tissues. Any sample containing a detectable amount of Ebola virus can be used. A sample can be a liquid such as urine, saliva, cerebrospinal fluid, blood, serum or the like; a solid or semi-solid such as tissues, feces, or the like; or, alternatively, a solid tissue such as those commonly used in histological diagnosis.

### In Vivo Detection of Ebola Virus

In using antibodies for the in vivo detection of antigen, the detectably labeled antibody is given in a close which is diagnostically effective. The term “diagnostically effective” means that the amount of detectably labeled antibody is administered in sufficient quantity to enable detection of the site having the Ebola virus antigen for which the antibodies are specific.

The concentration of detectably labeled antibody which is administered should be sufficient such that the binding to Ebola virus is detectable compared to the background. Further, it is desirable that the detectably labeled antibody be rapidly cleared from the circulatory system in order to give the best target-to-background signal ratio.

As a rule, the dosage of detectably labeled antibody for in vivo diagnosis will vary depending on such factors as age, sex, and extent of disease of the individual. The dosage of antibody can vary from about 0.01 mg/kg to about 50 mg/kg, e.g., 0.1 mg/kg to about 20 mg/kg, or about 0.1 mg/kg to about 2 mg/kg. Such dosages may vary, for example, depending on whether multiple injections are given, on the tissue being assayed, and other factors known to those of skill in the art.

For in vivo diagnostic imaging, the type of detection instrument available is a major factor in selecting an appropriate radioisotope. The radioisotope chosen must have a type of decay which is detectable for the given type of instrument, Still another important factor in selecting a radioisotope for in vivo diagnosis is that the half-life of the radioisotope be long enough such that it is still detectable at the time of maximum uptake by the target, but short enough such that deleterious radiation with respect to the host is acceptable. Ideally, a radioisotope used for in vivo imaging will lack a particle emission but produce a large number of photons in the 140-250 keV range, which may be readily detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions are the bifunctional chelating agents such as diethylenetriaminepentacetic acid (DTPA) and ethylenediaminetetra-acetic acid (EDTA) and similar molecules, Typical examples of metallic ions which can be bound to antibodies are ¹¹¹In, ⁹⁷Ru, ⁶⁷Ga, ⁶⁸Ga, ⁷²As, ⁸⁹Zr and ²⁰¹Tl.

The antibodies can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include ¹⁵⁷Gd, ⁵⁵Mn, ¹⁶²Dy, ⁵²Cr and ⁵⁸Fe.

The antibodies can be used in vitro and in vivo to monitor the course of Ebola virus therapy. Thus, for example, by measuring the increase or decrease in the number of cells infected with Ebola virus or changes in the concentration of Ebola virus present in the body or in various body fluids, it would be possible to determine whether a particular therapeutic regimen aimed at ameliorating Ebola virus is effective.

### Prophylaxis and Therapy of Ebola Virus Disease

The antibodies, polypeptides or nucleic acid molecules described herein can also be used in prophylaxis and as therapy for Ebola virus in both humans and other animals. The terms, “prophylaxis” and “therapy” as used herein in conjunction with the antibodies, polypeptides or nuclenic acid molecules described herein denote both prophylactic as well as therapeutic administration and both passive immunization with substantially purified polypeptide products, as well as gene therapy by transfer of polynucleotide sequences encoding the product or part thereof. Thus, the antibodies, polypeptides or nucleic acid molecules described herein can be administered to high-risk subjects in order to lessen the likelihood and/or severity of Ebola virus disease or administered to subjects already evidencing active Ebola virus infection.

As used herein, a “prophylactically effective amount” of the antibodies or polypeptides is a dosage large enough to produce the desired effect in the protection of individuals against Ebola virus infection for a reasonable period of time, such as one to two months or longer following administration. A prophylactically effective amount is not, however, a dosage so large as to cause adverse side effects, such as hyperviscosity syndromes, pulmonary edema, congestive heart failure, and the like. Generally, a prophylactically effective amount may vary with the subject's age, condition, and sex, as well as the extent of the disease in the subject and can be determined by one of skill in the art. The dosage of the prophylactically effective amount may be adjusted by the individual physician or veterinarian in the event of any complication. A prophylactically effective amount may vary from about 0.01 mg/kg to about 50 mg/kg, e.g., y from about 0.1 mg/kg to about 20 mg/kg, or from about 0.2 mg/kg to about 2 mg/kg, in one or more administrations (priming and boosting).

As used herein, a “therapeutically effective amount” of the antibodies, polypeptides or nucleic acid molecules described herein is a dosage large enough to produce the desired effect in which the symptoms of Ebola virus are ameliorated or the likelihood of infection is decreased. A therapeutically effective amount is not, however, a dosage no large as to cause adverse side effects, such as hyperviscosity syndromes, pulmonary edema, congestive heart failure, and the like. Generally, a therapeutically effective amount may vary with the subject's age, condition, and sex, as well as the extent of the disease in the subject and can be determined by one of skill in the art. The dosage of the therapeutically effective amount may be adjusted by the individual physician or veterinarian in the event of any complication. A therapeutically effective amount may vary from about 0.01 mg/kg to about 50 mg/kg, for instance from about 0.1 mg/kg to about 20 mg/kg, or from about 0.2 mg/kg to about 2 mg/kg, in one or more dose administrations daily, for one or several days. In one embodiment, administration of the antibody is for 2 to 5 or more consecutive days in order to avoid “rebound” of virus replication from occurring.

The antibodies or polypeptides, or isolated nucleic acid encoding the antibody or polypeptide, can be administered by injection or by gradual infusion over time. The administration of the antibodies, polypeptides or nucleic acid molecules described hereimay, for example, be intravenous, intraperitoneal, intramuscular, intracavity, subcutaneous, or transdermal. Techniques for preparing injectate or infusate delivery systems containing antibodies are well known to those of skill in the art.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution. Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and the like.

As described herein, Ebola virus binding antibodies or antigen-binding fragments, or varia thereof include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to neutralizing antibodies disclosed herein). ScFv molecules are known in the art and are described, e.g., in U.S. Pat. No. 5,892,019. Neutralizing antibody molecules can be of any type (e.g., IgG, IgE, 10/1, IgD, IgA, and IgY), class (e.g., IgG1, IgG2, IgG3, IgG4, IgA1 and IgA2) or subclass of immunoglobulin molecule.

Ebola virus neutralizing antibody fragments, including single-chain antibodies, may comprise the variable region(s) alone or in combination with the entirety or a portion of the following: hinge region, CH1. CH2, and CH3 domains. Also, Ebola virus neutralizing antigen-binding fragments can comprise any combination of variable region(s) with a hinge region, CH1, CH2, and CH3 domains.

### Humanization

In some embodiments, an animal antibody (e.g., rabbit antibody) can be modified, for example by, exchanging the Fe region with an Fe region from a different species (for example with a human Fc region). In some embodiments, one or more humanization changes also may be made (for example in one or more of the framework regions of the antibody).

In some embodiments, antibodies described herein may be engineered, by partial framework region replacement and sequence changing. In some embodiments. CDRs are derived from an antibody of a different class and/or a different species than the framework regions. In some embodiments, an engineered antibody contains one or more “donor” CDRs from a non-human antibody of known specificity that are grafted into a human heavy or light chain framework region. It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site, Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

EP 239 400 (Winter et al.) describes altering antibodies by substitution (within a given variable region) of their complementarity determining regions (CDRs) for one species with those from another. CDR-substituted antibodies are predicted to be less likely to elicit an immune response in humans compared to true chimeric antibodies because the CDR-substituted antibodies contain considerably less non-human components. (Riechmann et al. (1988); Verhoeyen et al. (1988)). Typically, CDRs of a murine antibody substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes are co-expressed in mammalian cells to produce soluble humanized antibody.

Queen et al. (1989) and WO 90/07861 have described a process that includes choosing human V framework regions by computer analysis for optimal protein sequence homology to the V region framework of the original murine antibody, and modeling the tertiary structure of the murine V region to visualize framework amino acid residues which are likely to interact with the murine CDRs. These murine amino acid residues are then superimposed on the homologous human framework. See also U.S. Pat. Nos. 5,693,762; 5,693,761; 5,585,089; and U.S. Pat. No. 5,530,101. Tempest et al. (1991) utilize, as standard, the V region frameworks derived from NEWM and REI heavy and light chains respectively for CDR-grafting without radical introduction of mouse residues. An advantage of using the Tempest et al., approach to construct NEWM and REI based humanized antibodies is that the three-dimensional structures of NEWM and REI variable regions are known from x-ray crystallography and thus specific interactions between CDRs and V region framework residues can be modeled. However, it should be appreciated that similar approaches may be based on one or more other known antibody structures (e.g., based on one or more Fab structures). In some embodiments, a human germline framework may be used (e.g., as described for antibody 399 herein).

Non-human antibodies can be modified to include substitutions that insert human immunoglobulin sequences, e.g., consensus human amino acid residues at particular positions, e.g., at one or more of the following positions (e.g., at least five, ten, twelve, or all); (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H5 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213.

### Applications

In some embodiments, an antibody, isolated polypeptide or isolated nucleic acid can be administered to a subject to prevent or treat an Ebola virus infection.

In some embodiments, aspects of the invention relate to compositions that inhibit Ebola virus activity, for example, that inhibit one or more of viral proliferation (e.g., viral replication) and infectivity. In some embodiments, such compositions can be used to treat or suppress conditions associated with Ebola virus activity in subjects that are infected with an Ebola virus, or to lower the risk of infection with the Ebola virus, Such compositions may be used to prevent viral infection, to prevent an increase in virus viral activity, to prevent virus proliferation, to prevent symptoms associated with viral infection, to treat a subject infected with a virus, or treat a subject at risk of infection with a virus, or to treat a subject that has developed a disease or condition associated with infection by a virus. Compositions of the invention also may be administered to a subject at risk of a viral infection or at risk of an increase in viral activity (e.g., viral proliferation), regardless of whether the subject is actually known to have been exposed to, or infected by, the virus.

In some embodiments, one or more compositions of the invention may be administered alone or in combination with other compositions described herein or along with other therapeutic agents. Compositions of the invention may be provided (e.g., administered) in pharmaceutical preparations. Compositions of the invention may be provided in kits.

In some embodiments, an isolated antibody, isolated polypeptide or isolated nucleic acid can be useful to slow the progression of an Ebola virus infection.

In some embodiments, the invention provides methods of inhibiting viral replication, the methods comprising contacting a cell comprising an Ebola virus with an isolated antibody, isolated polypeptide or isolated nucleic acid composition. In certain embodiments, the preparation is administered intravenously. In other embodiments, the preparation is administered orally. Alternative routes of administration include sublingual, intramuscular, and transdermal administrations. Accordingly, preparations of the present invention may be given orally, parenterally, topically, or rectally. They are of course given in forms suitable for each administration route.

The compositions may be administered to humans and other animals for therapy by any suitable route of administration. Actual dosage levels may be adjusted to obtain an amount that is effective to achieve the desired therapeutic response for a particular patient, composition, and mode of administration, without being toxic to the patient.

The selected dosage level will depend upon a variety of factors including the activity of the isolated antibody, isolated polypeptide or isolated nucleic acid the clearance rate of the isolated antibody, isolated polypeptide or isolated nucleic acid, the duration of the treatment, other drugs, compounds and/or materials used in combination with the particular isolated antibody, isolated polypeptide or isolated, the age, sex, weight, condition, general health and prior medical history of the patient being treated, and like factors well known in the medical arts. Such an effective dose will generally depend upon the factors described above. In some embodiments, at least 0.5-1 mg/kg may be used. However, higher or lower amounts may be used. In some embodiments, an effective dose of an antibody or polypeptide described herein may be about 100 mg/kg or more. In some embodiments, 300 to 600 mg/kg may be used.

A physician or veterinarian having ordinary skill in the art can readily determine and prescribe the effective amount of the isolated antibodies, isolated polypeptides or isolated nucleic acid molecules described herein. For example, the physician or veterinarian could start doses of the compositions at levels lower than that required to achieve the desired therapeutic effect and then gradually increasing the dosage until the desired effect is achieved.

For example, antibody preparations may be formulated for administration in any convenient way for use in human or veterinary medicine, by analogy with other antibodies. In some embodiments, aspects of the invention also relate to a method of making a medicament for use in treating a subject, e.g., for treating or preventing an Ebola virus infection, or for inhibiting Ebola virus replication or proliferation. Such preparations can be used for prophylactic treatment of a subject at risk for or suspected of having an Ebola virus infection. Accordingly, one or more antibody compositions described herein that modulate virus replication or proliferation as described herein may be used for the preparation of a medicament for use in any of the methods of treatment described herein. In some embodiments, the invention provides for the use of one or more antibody compositions of the invention for the manufacture of a medicament or pharmaceutical for treating a mammal (e.g., a human) having one or more symptoms of, or at risk for, Ebola virus infection, replication and/or proliferation. Accordingly, the invention also relates to one or more antibody compositions described herein for use as a medicament. The invention also relates to one or more of these antibody compositions for use in methods described herein, for example in methods of inhibiting replication, or of treating or preventing a disease associated with Ebola virus replication or proliferation.

Antibodies can be prepared in a physiologically acceptable formulation and may comprise a pharmaceutically acceptable carrier, diluent and/or excipient using known techniques. Suitable pharmaceutical carriers, diluents and/or excipients are well known in the art and include, for example, phosphate buffered saline solutions, water, emulsions such as oil/water emulsions, various types of wetting agents, sterile solutions, etc.

Formulation of the pharmaceutical composition according to the invention can be accomplished according to standard methodology know to those of ordinary skill in the art.

The compositions of the present invention may be administered to a subject in the form of a solid, liquid or aerosol at a suitable, pharmaceutically effective dose. Examples of solid compositions include pills, creams, and implantable dosage units. Pills may be administered orally. Therapeutic creams may be administered topically. Implantable dosage units may be administered locally, for example, at a tumor site, or may be implanted for systematic release of the therapeutic composition, for example, subcutaneously, Examples of liquid compositions include formulations adapted for injection intramuscularly, subcutaneously, intravenously, intra-arterially, and formulations for topical and intraocular administration, Examples of aerosol formulations include inhaler formulations for administration to the lungs.

The compositions may be administered by standard routes of administration. In general, the composition may be administered by topical, oral, rectal, nasal, interdermal, intraperitoneal, or parenteral (for example, intravenous, subcutaneous, or intramuscular) routes. In addition, the composition may be incorporated into sustained release matrices such as biodegradable polymers, the polymers being implanted in the vicinity of where delivery is desired, for example, at the site of a tumor. The method includes administration of a single dose, administration of repeated doses at predetermined time intervals, and sustained administration for a predetermined period of time.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. In one embodiment, the biodegradable matrix is a matrix of one of either polylactide, polyglycolide, or polylactide co-glycolide (co-polymers of lactic acid and glycolic acid).

It is well known to those of ordinary skill in the art that the dosage of the composition will depend on various factors such as, for example, the condition of being treated, the particular composition used, and other clinical factors such as weight, size, sex and general health condition of the patient, body surface area, the particular compound or composition to be administered, other drugs being administered concurrently, and the route of administration.

Proteinaceous pharmaceutically active matter may be present in amounts between 1 ng and 10 mg per dose. Generally, the regime of administration should be in the range of between 0.1 μg and 10 mg of the antibody according to the invention, particularly in a range 1.0 μg to 1.0 mg, and more particularly in a range of between 1.0 μg and 100 μg, with all individual numbers falling within these ranges also being part of the invention. If the administration occurs through continuous infusion a more proper dosage may be in the range of between 0.01 μg and 10 mg units per kilogram of body weight per hour with all individual numbers falling within these ranges also being part of the invention.

Preparations for parenteral administration include sterile aqueous or non-aqueous solutions, suspensions and emulsions, Non-aqueous solvents include without being limited to it, propylene glycol, polyethylene glycol, vegetable oil such as olive oil, and injectable organic esters such as ethyl oleate, Aqueous solvents may be chosen from the group consisting of water, alcohol/aqueous solutions, emulsions or suspensions including saline and buffered media. Parenteral vehicles include sodium chloride solution, Ringers dextrose, dextrose and sodium chloride, lactated Ringers, or fixed oils, Intravenous vehicles include fluid and nutrient replenishers, electrolyte replenishers (such as those based on Ringers dextrose) and others. Preservatives may also be present such as, for example, antimicrobials, anti-oxidants, chelating agents, inert gases, etc.

### Diagnostic Applications and Kits:

In some embodiments, antibodies or polypeptides described herein can be used as detection reagents for in vivo diagnostics, and/or coupled to contrast dye reagents for radiology,

In some embodiments, aspects of the invention include using immobilized or non-immobilized, anti-Ebola virus antibodies or polypeptides as detection moieties to assess the presence and/or level of Ebola virus in a sample. Detection assays may include the use of one or more labeled detection moieties (VP-binding antibody containing or attached to a detectable label). A detectable label is defined as any moiety that can be detected using an assay. The antibodies and functional antibody fragments can be coupled to specific labeling agents for detecting binding according to standard coupling procedures. A wide variety of detectable labels can be used, such as those that provide direct detection (e.g., a radioactive label, a fluorophore, [e.g. Green Fluorescent Protein (GFP), Red Fluorescent Protein (RFP), etc.), a chromophore, an optical or electron dense label, etc.) or indirect detection (e.g., an enzyme tag such as horseradish peroxidase, etc.). Non-limiting examples of detectable labels that have been attached to or incorporated into antibodies include: enzymes, radiolabels, fluorescent labels, phosphorescent molecules, chemiluminescent molecules, chromophores, luminescent molecules, photoaffinity molecules, and colored particles or ligands such as biotin, etc. In some embodiments, detection methods of the invention may include electrochemiluminescence methods (ECL).

A variety of methods may be used to detect a label, depending on the nature of the label and other assay components. Labels may be directly detected through optical or electron density, radioactive emissions, non-radiative energy transfers, etc. or indirectly detected with antibody conjugates, streptavidin-biotin conjugates, etc. Many additional detectable labels are known in the art, as are methods for their attachment to antibodies.

Labeled antibodies or polypeptides may be used in vitro, e.g., in an immunoassay such as an ELISA. Such detectably labeled antibodies or polypeptides that have a detectable label incorporated into the antibody or polypeptide or may be linked to a secondary binding ligand and/or to an enzyme (an enzyme tag) that will generate a detectable (e.g., colored) product upon contact with a chromogenic substrate, Examples of suitable enzymes include, but are not limited to, urease, alkaline phosphatase, (horseradish) hydrogen peroxidase or glucose oxidase. Examples of suitable secondary binding ligands include, but are not limited to, biotin and/or avidin and streptavidin compounds. The use of such labels is well known to those of skill in the art and is described, for example, in U.S. Pat. Nos. 3,817,837; 3,850,752; 3,939,350; 3,996,345; 4,277,437; 4,275,149 and 4,366,241; each incorporated herein by reference.

Numerous methods for the attachment or conjugation of an antibody or polypeptide to its detectable label are known in the art. An attachment method may include the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3alpha-6alpha-diphenylglycouril-3 attached to the antibody (see, for example, U.S. Pat. Nos. 4,472,509 and 4,938,948, each incorporated herein by reference). Antibodies or parts thereof also can be reacted with an enzyme in the presence of a coupling agent such as glutaraldehyde or periodate. Antibodies may be labeled with fluorescein markers in the presence of these coupling agents or by reaction with an isothiocyanate. In other embodiments, antibodies may be labeled by derivatization, for example, by selectively introducing sulfhydryl groups in the Fc region of the antibody, using reaction conditions that do not alter the antibody recognition site.

Detection of a detectable label in an assay of the invention is also referred to herein as detecting the “signal” Methods for detecting the signal in an immunoassay are well known in the art. In some embodiments, an assay signal can be detected using a multi-well plate reader (e.g., microplate reader) to assess the amount and/or location of a signal, Signal detection can be optical detection or other detection means suitable for detecting a detectable label utilized in the invention.

The invention will be further described by the following non-limiting example.

### Example

### Materials and Methods

Viruses and Cells.

Ebola virus species Zaire, strain Mayinga (wild type), and a mouse-adapted Ebola virus strain were propagated in Vero E6 cells and stored at −80° C. until use. VSV pseudotyped with Ebola virus GP (VSVΔG*-EbolaGP) expressing green fluorescent protein (GFP) was generated as previously described (Takada et al., 1997). Human embryonic kidney 293 cells were grown in Dulbecco modified Eagle medium complemented with 10% fetal bovine serum, L-glutamine, and antibiotics. VSV genomic plasmid pVSV-XN2 and plasmids for nucleoprotein, polymerase, and phosphoprotein expression were kindly provided by J. Rose, Yale University, New Haven, Conn. A recombinant VSV containing the Ebola virus GP-encoding gene instead of the VSV G protein-encoding gene (chimeric VSV-EbolaGP) was generated as follows. The open reading frame of the Ebola virus GP-encoding gene was cloned into plasmid pVSV-XN2 lacking the VSV G protein-encoding gene (VSV-AG) at the site where the VSV G protein-encoding gene was deleted. The recombinant VSV expressing Ebola virus GP instead of VSV G protein (chimeric VSV-EbolaGP) was then generated as previously described (Schnell et al., 1996). The virus was propagated in Vero E6 cells, and its titer was determined by plaque assay (10⁷ PFU/mL). A characterization of the recombinant virus will be published elsewhere. All infectious materials involving chimeric VSV-EbolaGP were handled in a biosafety level 4 facility at the Canadian Science Centre for Human and Animal Health.

MAbs.

MAbs were produced as described previously (Takada et al., 2001). The hybridomas producing MAbs 133/3.16 (immunoglobulin G1 [IgG1]), 226/8.1 (IgG1), and 42/3.7 (IgG1) were grown in PFHM II (GIBCO BRL), and the antibodies were purified from the supernatants with protein A agarose columns (Bio-Rad). Mouse ascites was obtained by a standard procedure, and the concentration of GP-specific antibodies in the ascites was determined by enzyme-linked immunosorbent assay (ELISA) by using the purified antibodies as standards.

Virus Neutralization Tests of VSV Pseudotyped with Ebola Virus GP and Ebola Virus.

VSVΔG*-EbolaGP or Ebola virus species Zaire was incubated with MAbs for 1 hour at room temperature and inoculated onto monolayers of 293 cells. Infectivities of the viruses were determined by counting the fluorescent cells as described previously (Takada et al., 1997). The relative percentage of infected cells was determined by setting the number of infected cells in the presence of normal mouse IgG or ascites (approximately 50 to 100 fluorescent cells per microscopic field) to 100.

Immunofluorescence Assay.

293 cells infected with Ebola virus were fixed with 2% paraformaldehyde 1 day after infection and treated with 0.1% Triton X-100 in phosphate-buffered saline, To detect virus-infected cells, rabbit antiserum to VP40 of Ebola virus (Jasenosky et al., 2001) was used as the primary antibody. Goat anti-rabbit IgG conjugated with fluorescein isothiocyanate was purchased from Sigma (St. Louis, Mo.).

Selection of Escape Mutants.

Tenfold dilutions of chimeric VSV-EbolaGP were incubated with appropriately diluted mouse ascites (250 to 500 μg of specific antibodies/ml) at room temperature for 1 hour, and the mixtures were inoculated onto Vero E6 cells. Mutant viruses that grew in the presence of the MAbs were harvested from the highest dilution of the virus. This procedure was repeated, After confirming the growth of the virus in the presence of the antibodies, the viral RNA was extracted and the nucleotide TO sequences of the GP-encoding genes determined by standard procedures.

Passive Immunization and Protection Tests with Mice.

Five-week-old female BALB/c mice (Charles River) were given 100 μL of appropriately diluted ascites (250 μg of specific antibodies/mouse) intraperitoneally on days—1 and 2. On day 0, all mice were intraperitoneally infected with 30 50% lethal doses of the mouse-adapted Ebola virus strain. The mice were monitored for clinical signs of infection for 24 days after the challenge.

Results

Specificity of MAbs.

VSV pseudotyped with GP from species Zaire was first used for virus neutralization tests and found that of the 10 clones we generated, two MAbs, 133/3.16 (IgG1) and 226/8.1 (IgG1), neutralized the infectivity of the virus. Then it was confirmed that authentic Ebola virus species Zaire infectivity was also neutralized by these antibodies. MAb 42/3.7 recognized GPs from all of the Ebola virus species in an ELISA (data not shown) but did not neutralize virus infectivity. While both MAbs 133/3.16 and 226/8.1 efficiently neutralized the infectivity of VSV pseudotyped with GP from species Zaire, neither of these MAbs appreciably neutralized the infectivity of the virus pseudotyped with GPs from the other Ebola virus species, Sudan, Ivory Coast, and Reston. Limited cross-neutralizing activity was found with MAb 133/3.16 when the viruses were treated with the antibody at a higher concentration (100 μg/mL). The species specificity of these MAbs was also confirmed by ELISA with cells transfected with plasmids expressing these GPs (data not shown).

Protective Effects of Passive Immunization of Mice with Neutralizing Antibodies.

Next, the protective potential of the neutralizing antibodies was tested in a mouse model (Table 1). Mice were treated with the antibodies twice, 1 day prior to and 2 days after a challenge with the mouse-adapted Ebola virus strain (Bray et al., 1998). All mice treated with either MAb 133/3.16 or 226/8.1 were protected from a lethal infection without disease signs, while untreated mice and those treated with MAb 42/3.7, which lacks virus-neutralizing activity, lost weight and died by day 8 post-challenge,

TABLE 1
Protection conferred by passive immunization
of mice with neutralizing antibodies^{a}

| Antibody | No. of survivors/total |
|---|---|
| 133/3.16 | 6/6 |
| 226/8.1 | 6/6 |
| 42/3.7b | 0/5 |
| None | 0/7 |

^{a}Each mouse was intraperitoneally inoculated with 250 μg of the indicated antibody 1 day before and 2 days after an intraperitoneal challenge with 30 50% lethal doses of Ebola virus.
bThis antibody reacts with the GPs of all Ebola virus species but does not neutralize virus infectivity in vitro.

Identification of Neutralizing Epitopes with Chimeric GPs.

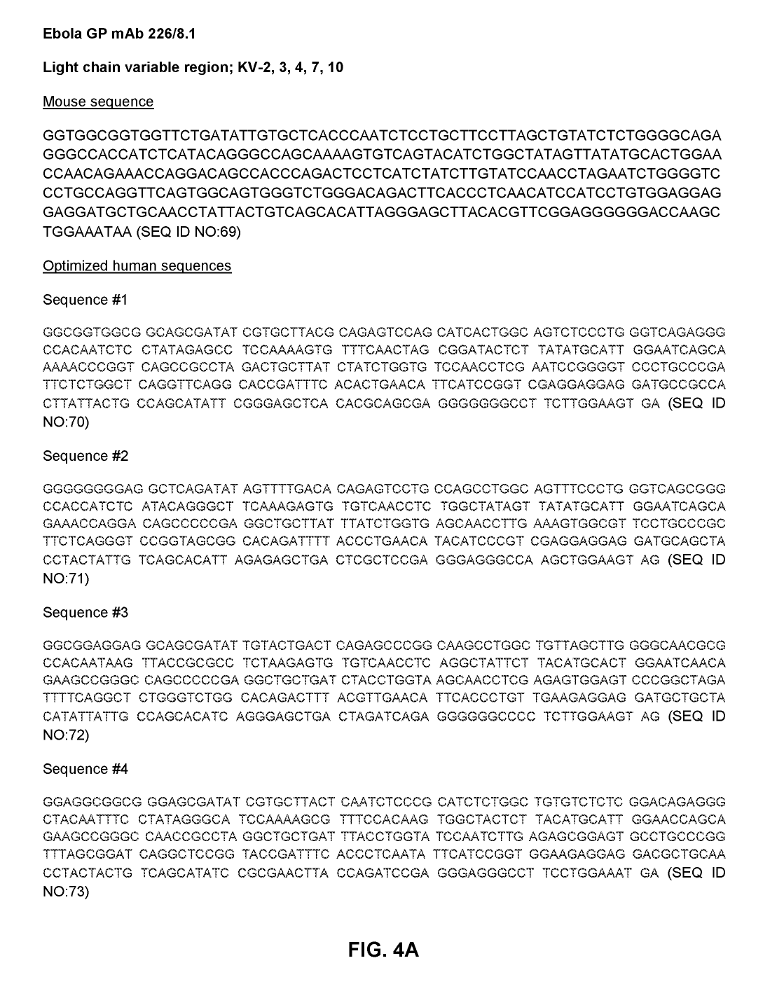

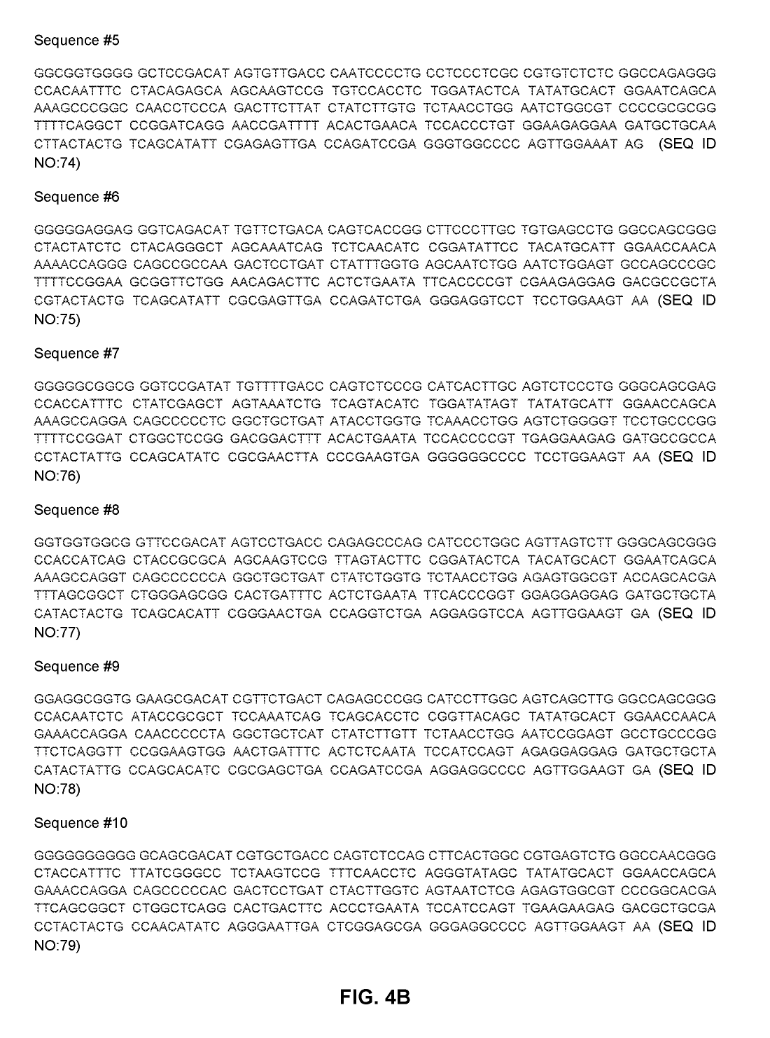

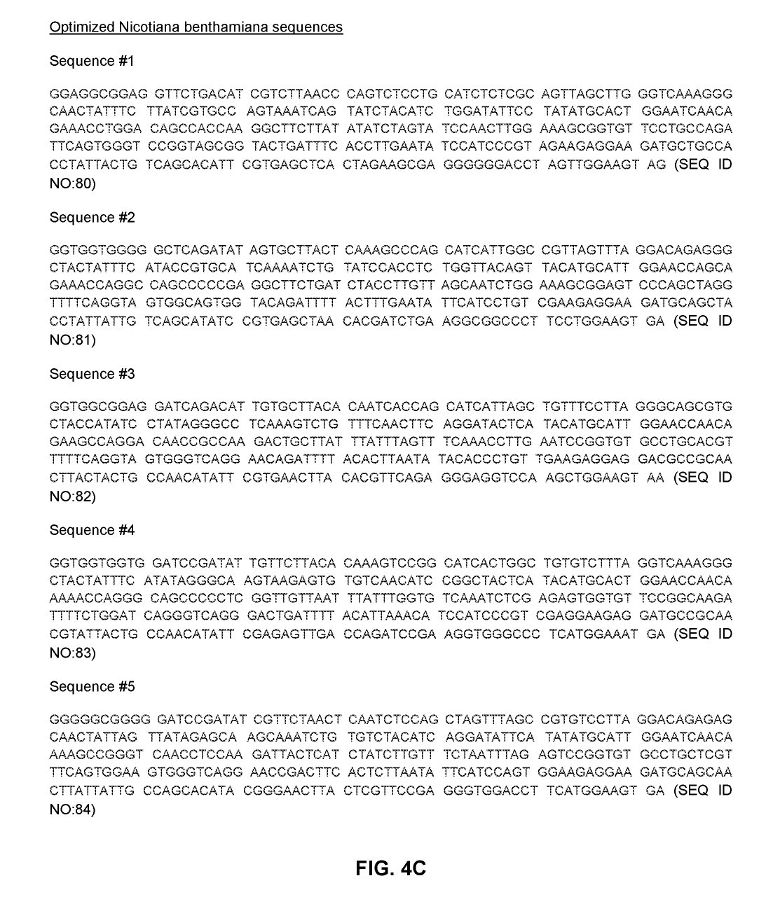

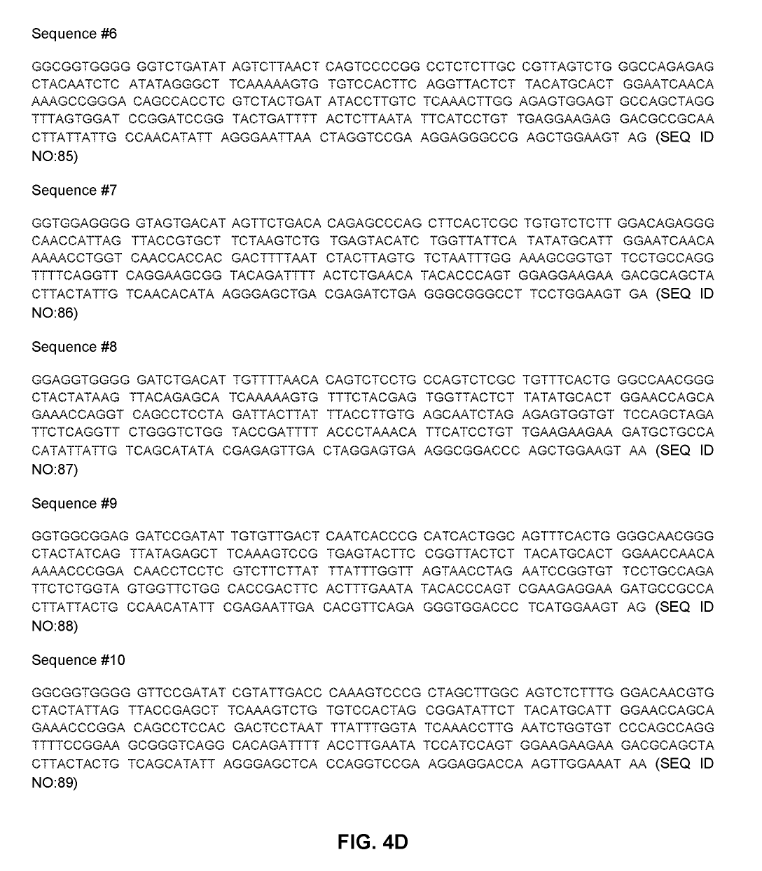

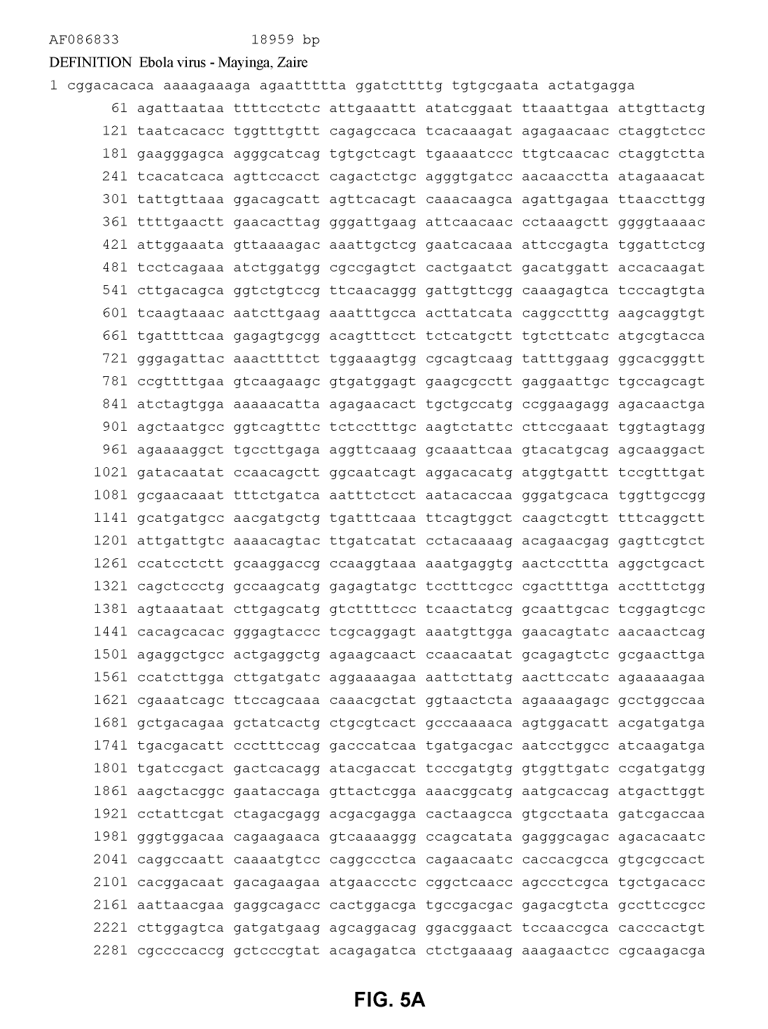

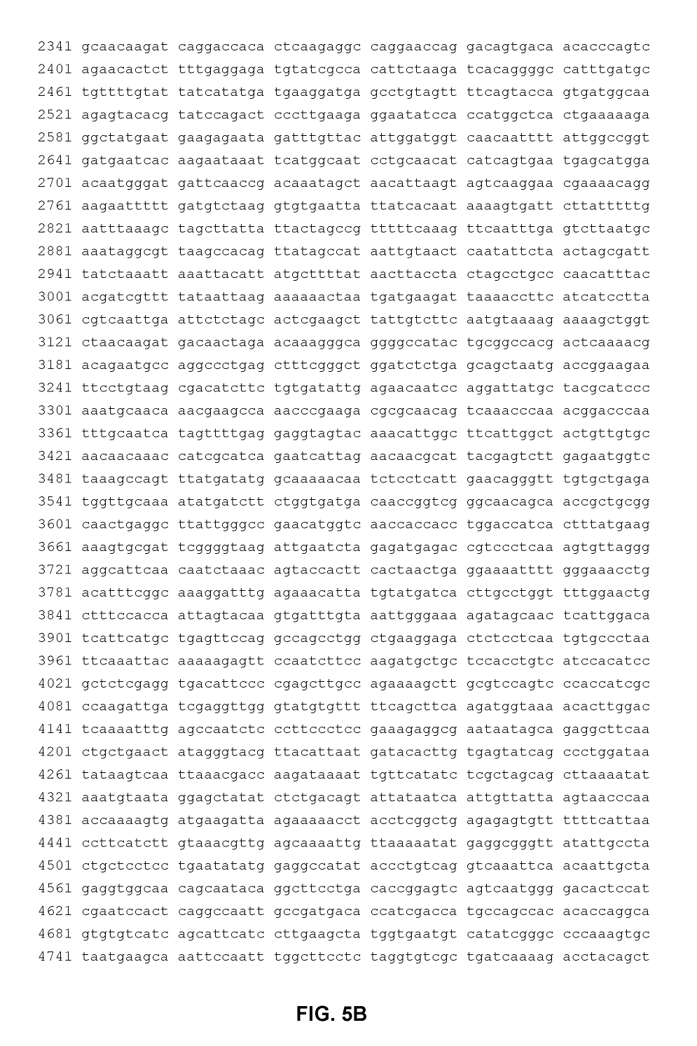

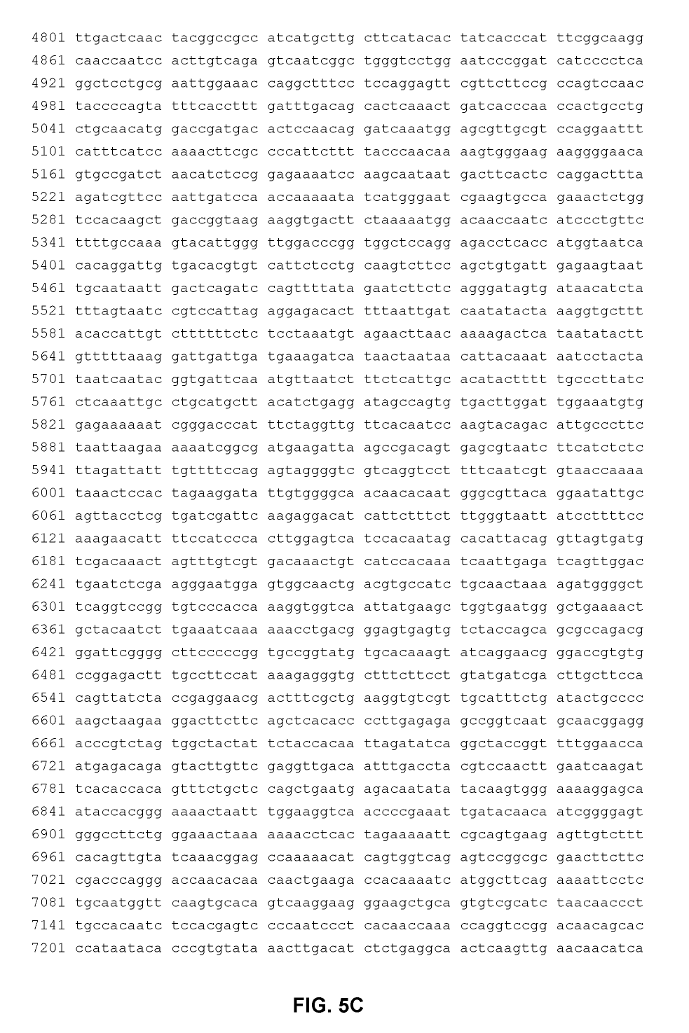

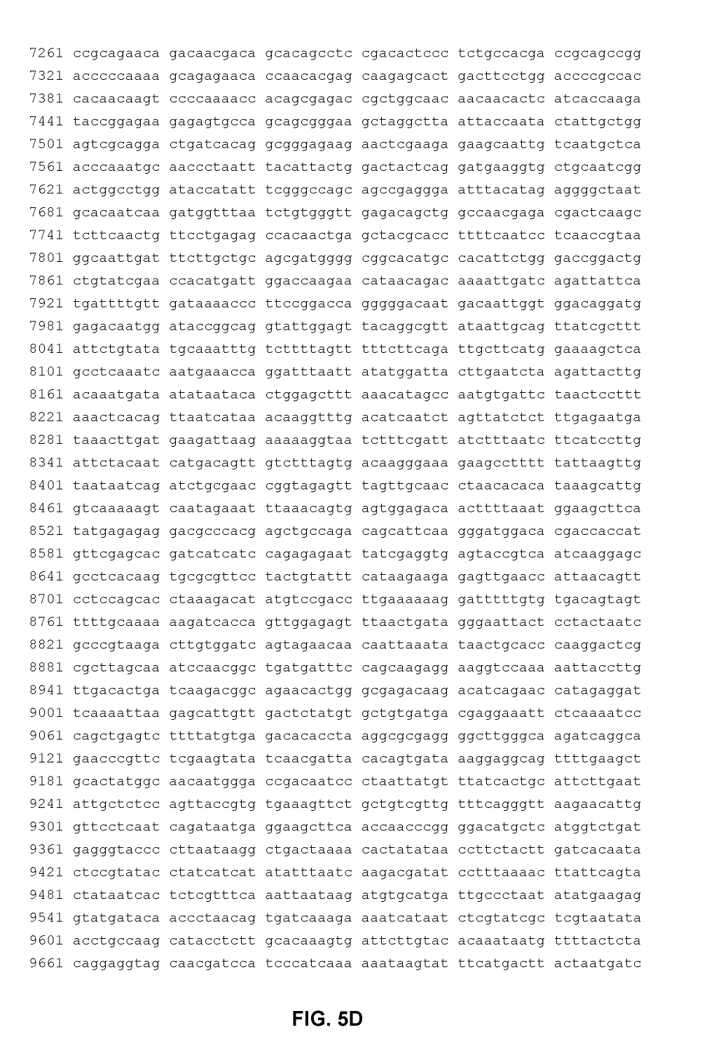

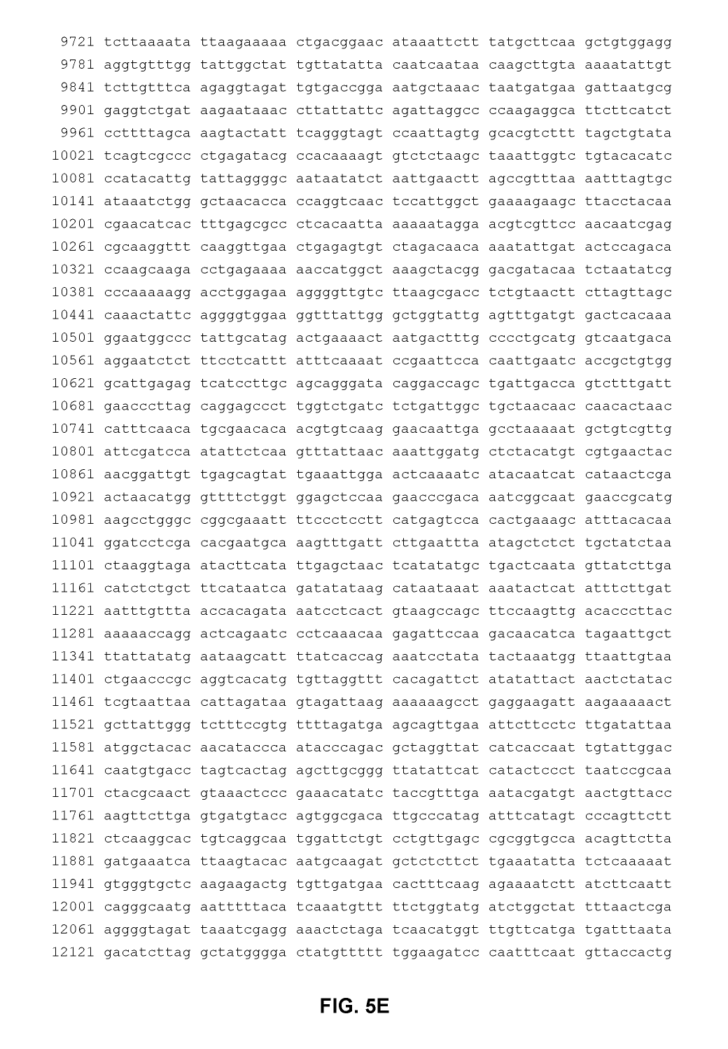

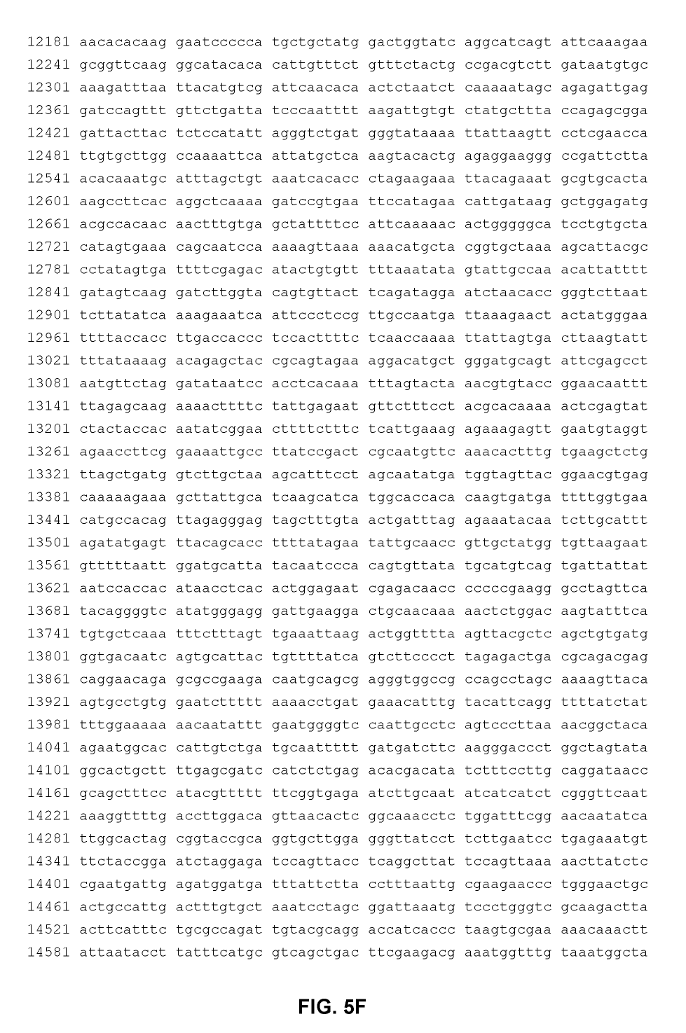

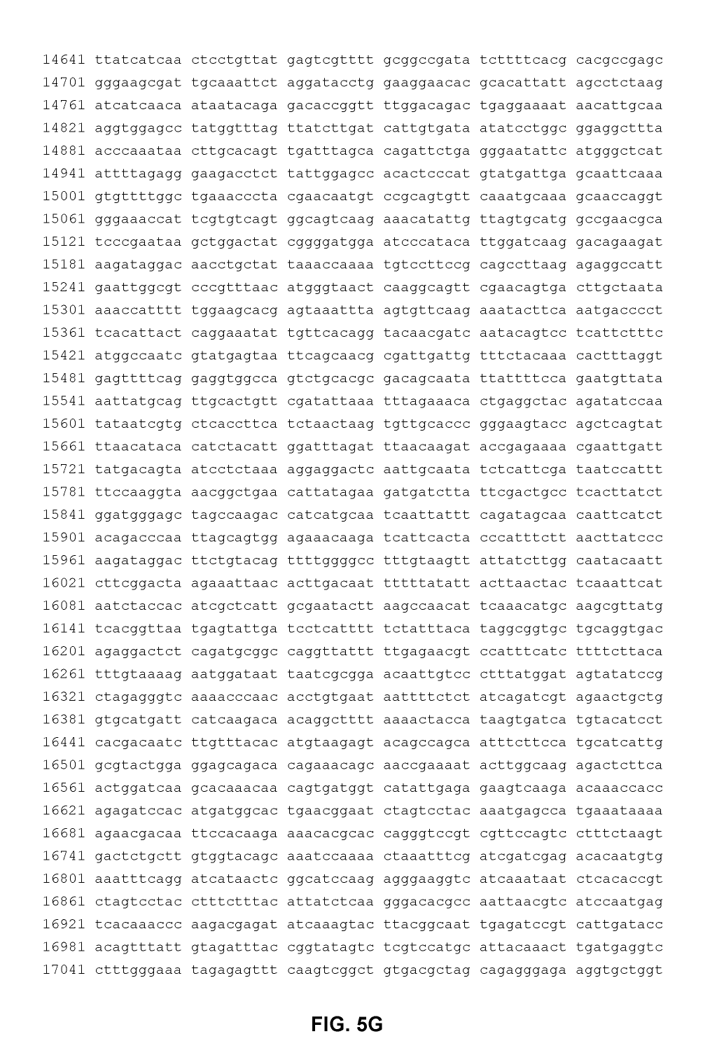

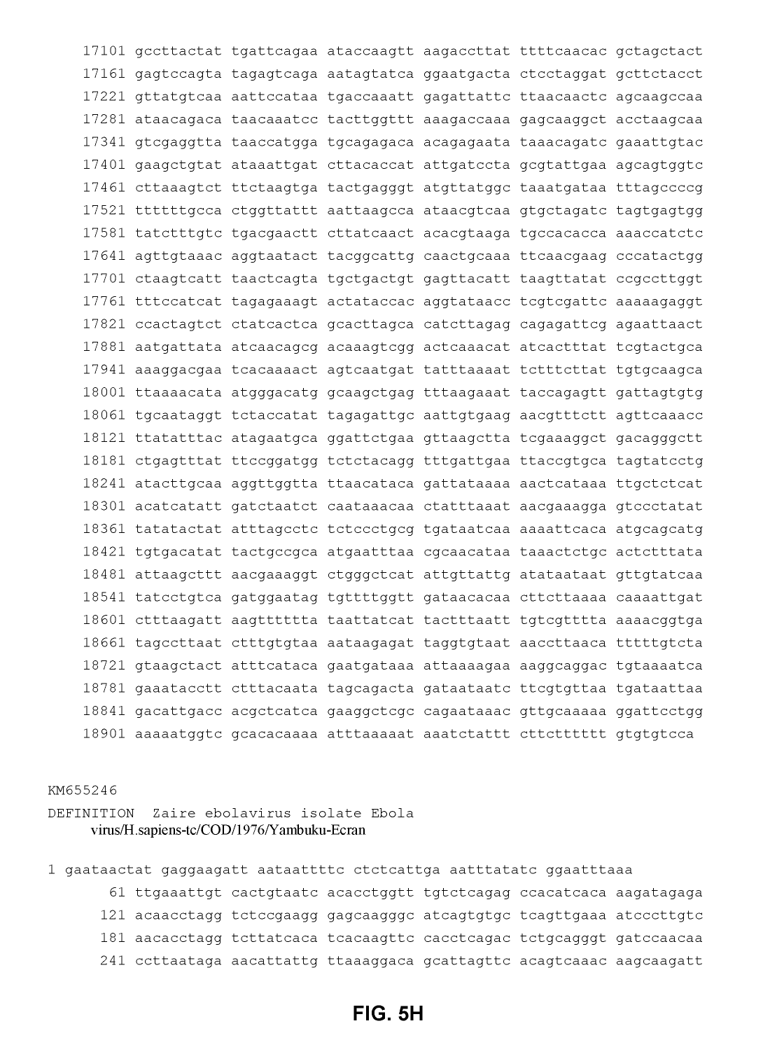

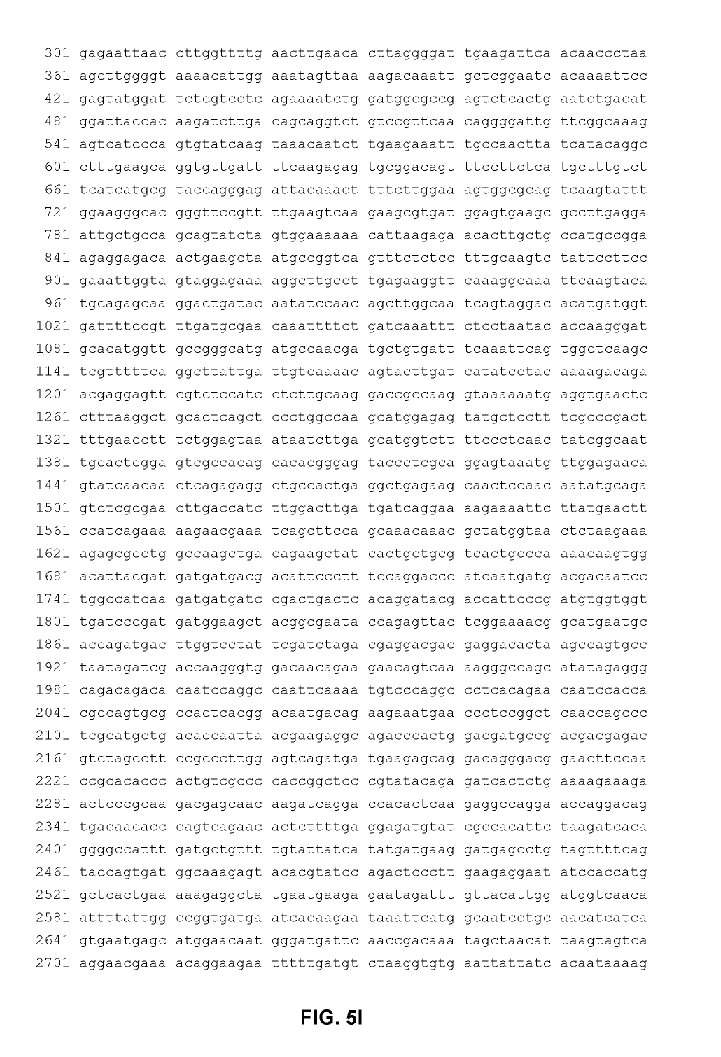

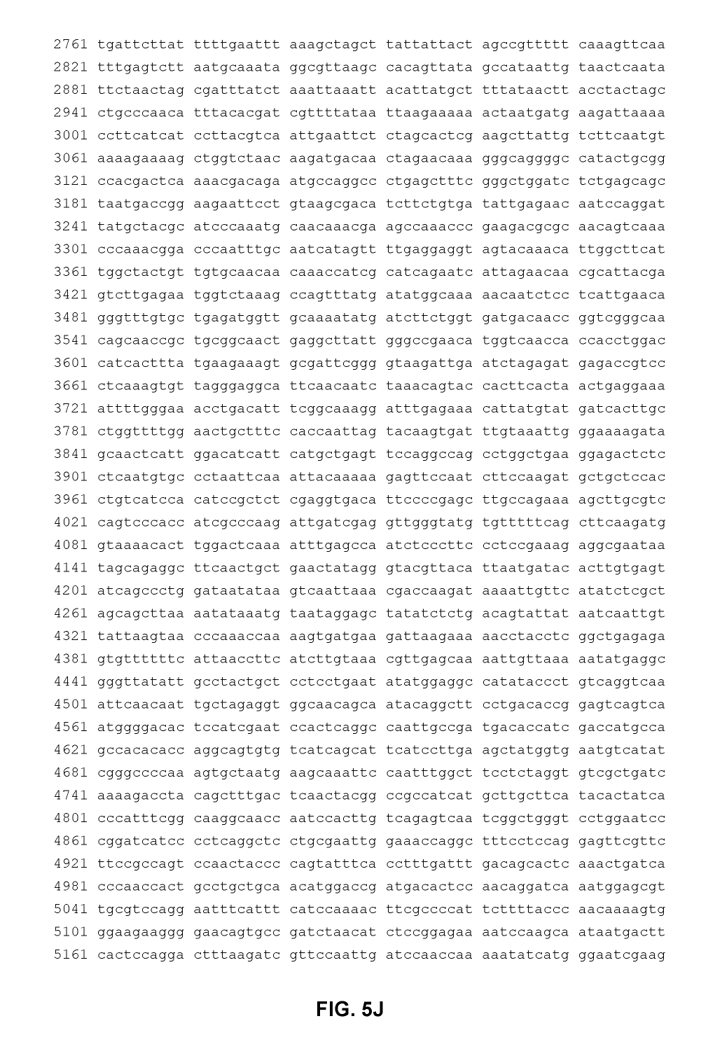

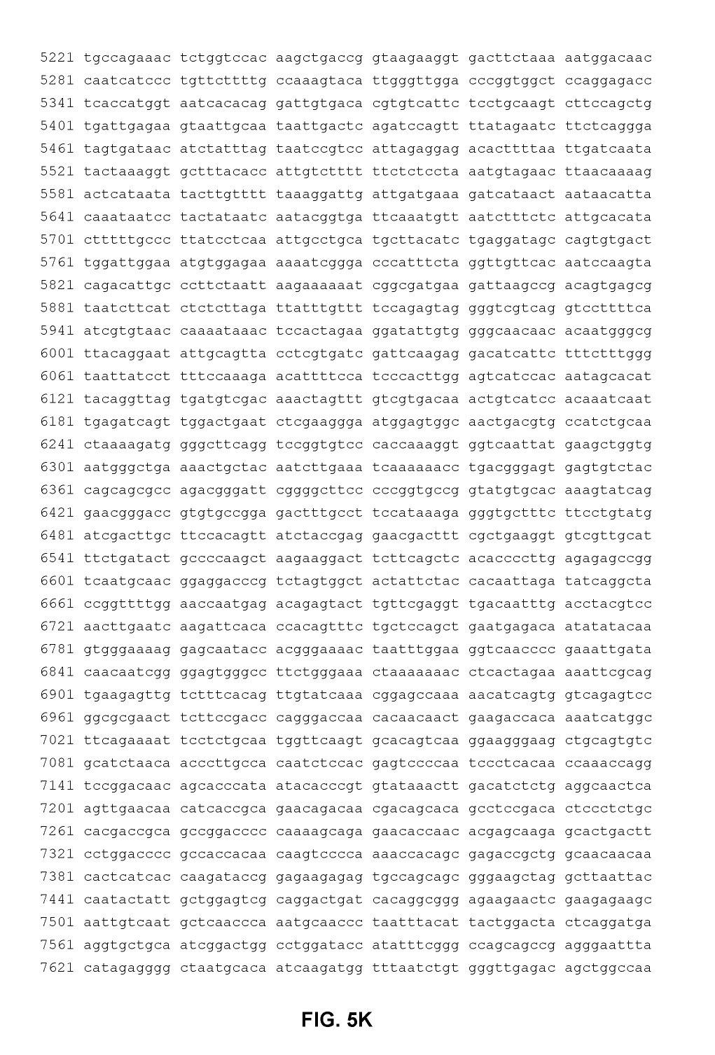

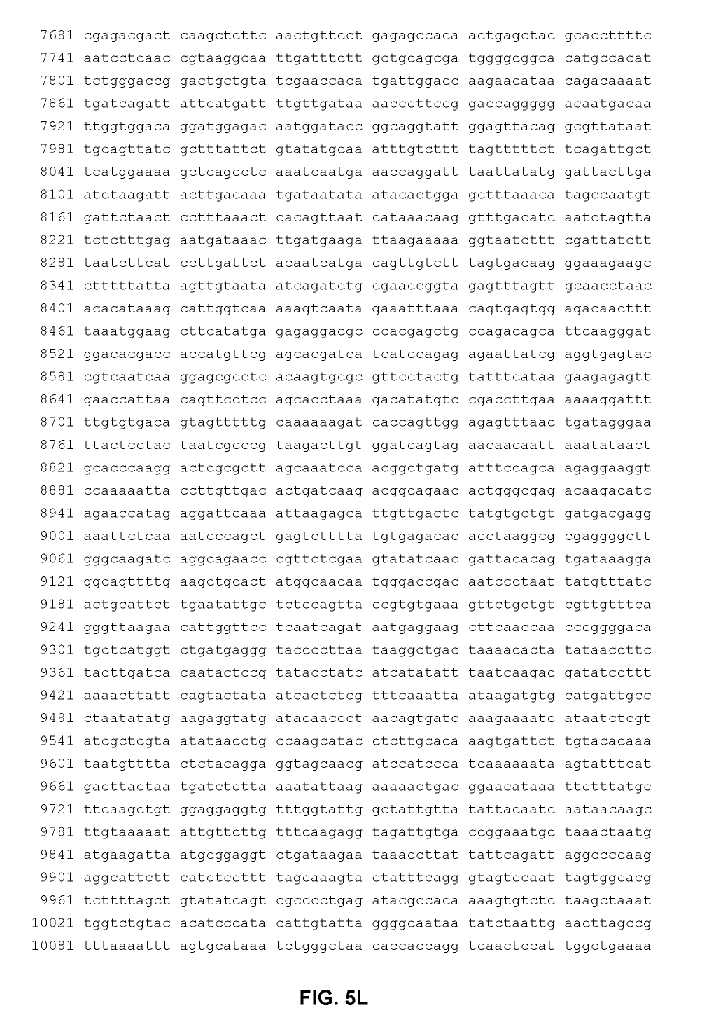

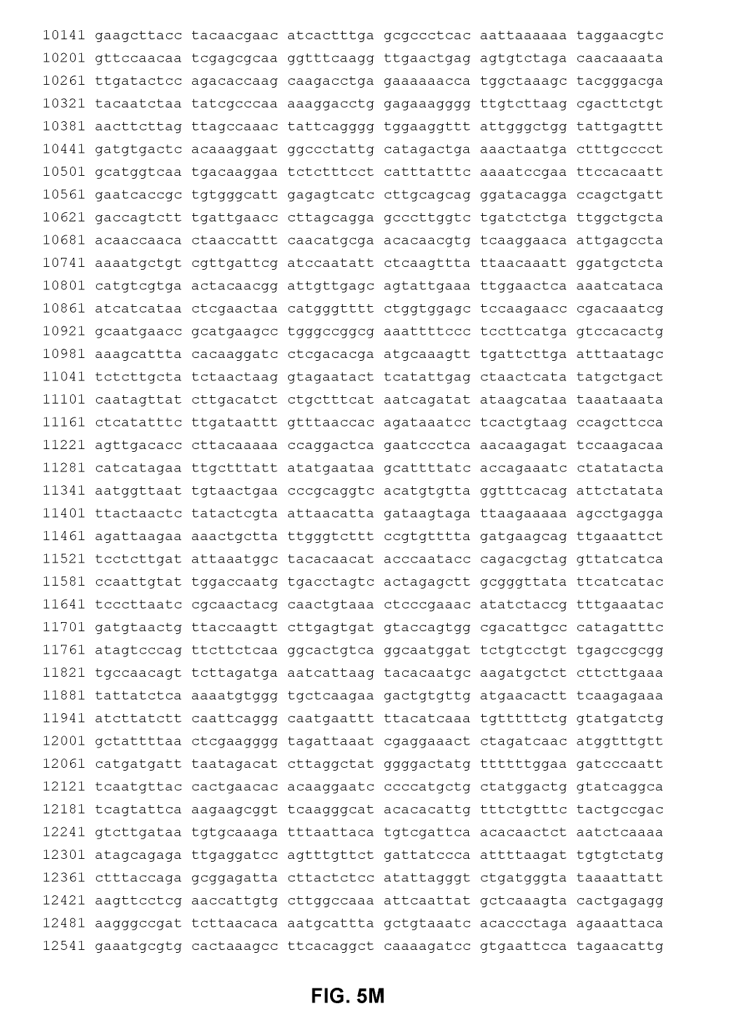

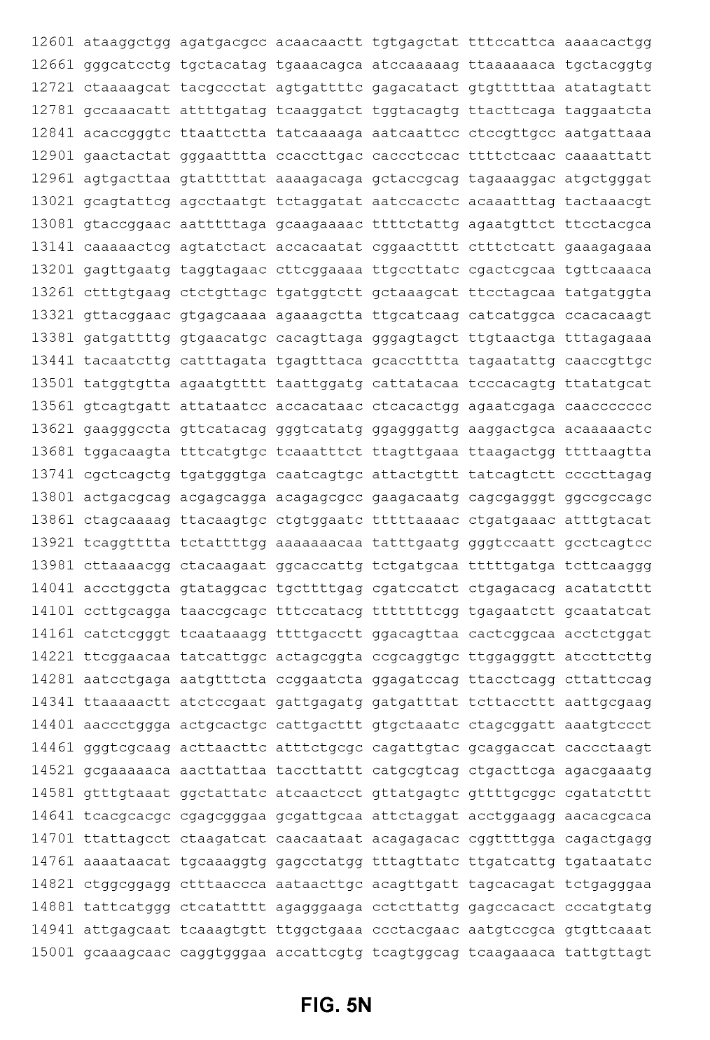

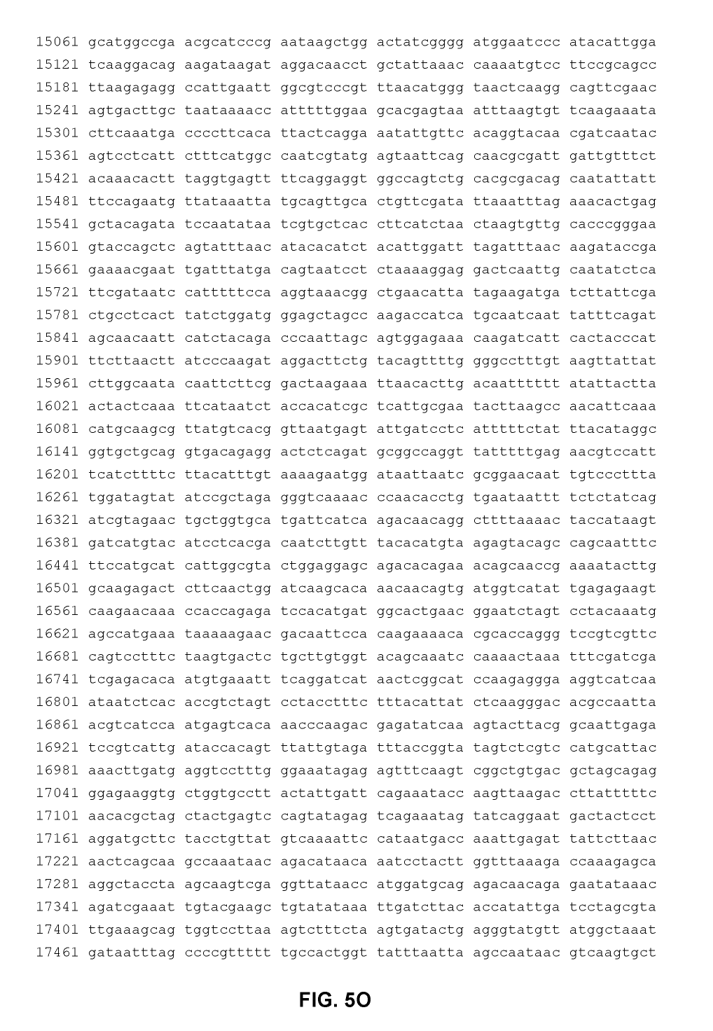

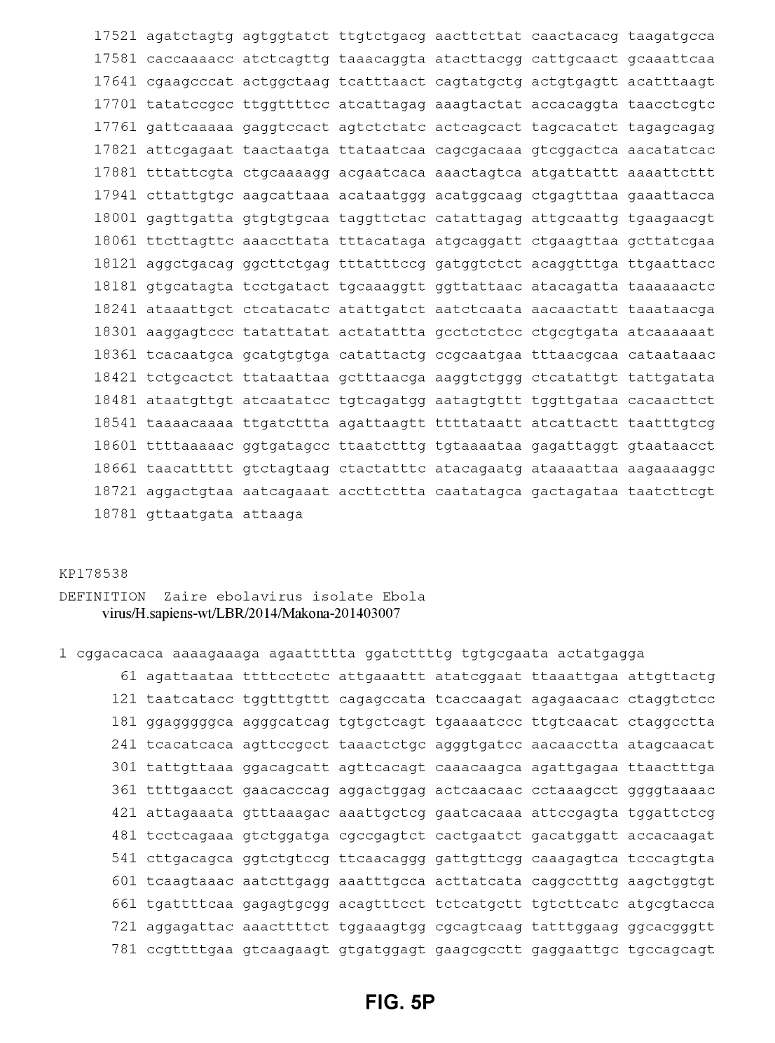

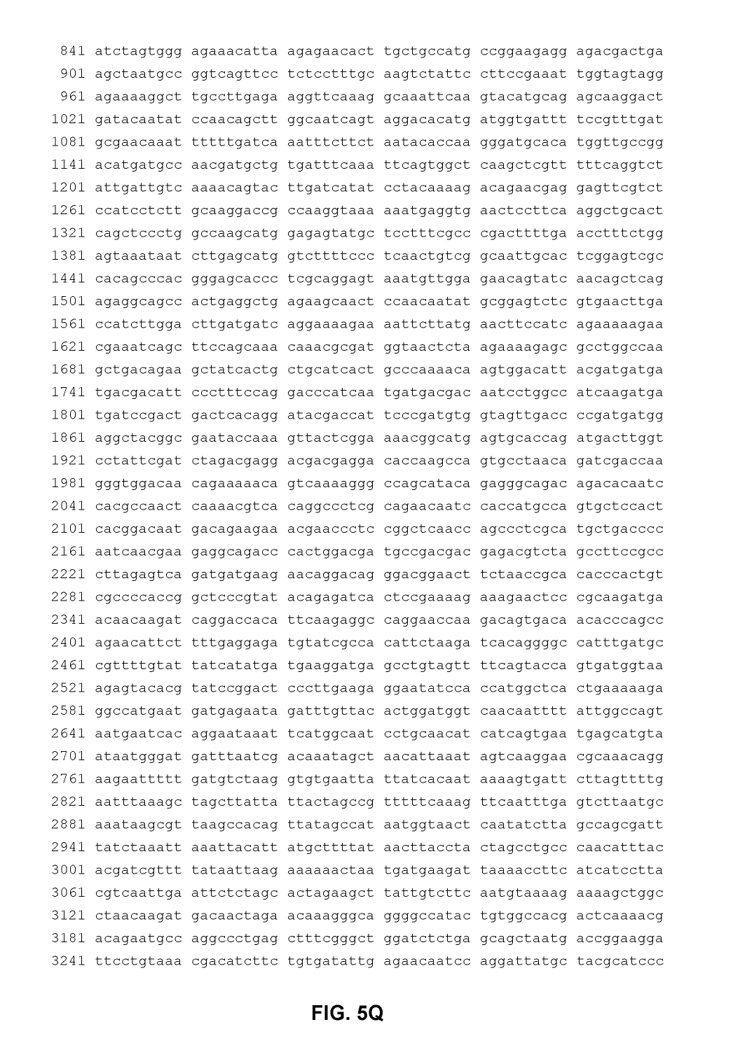

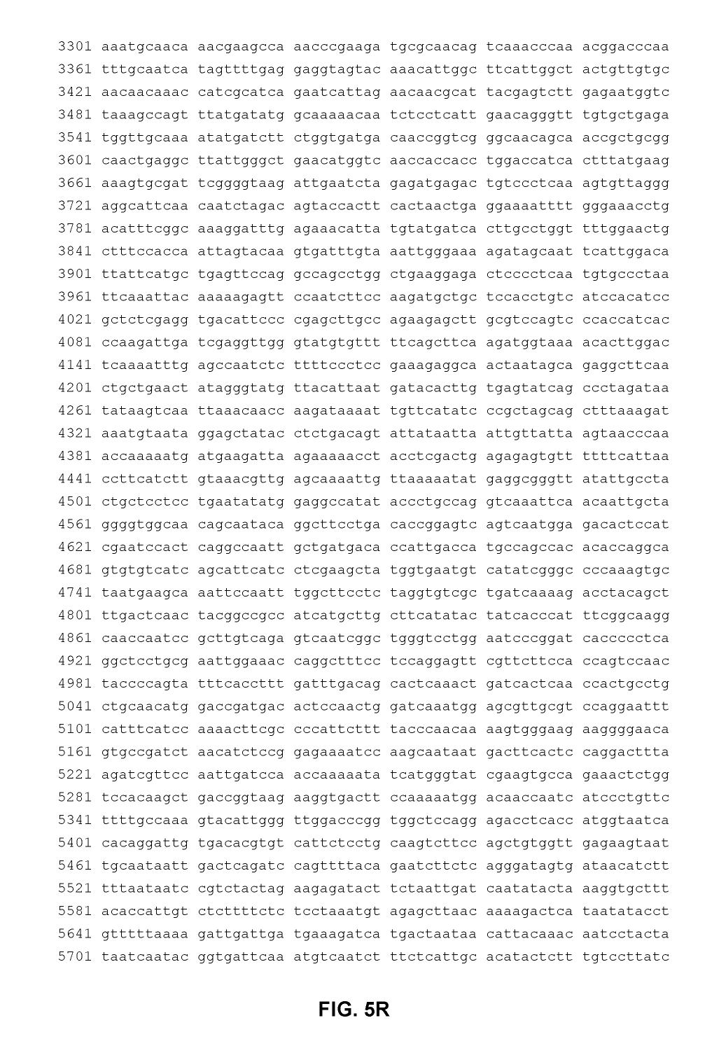

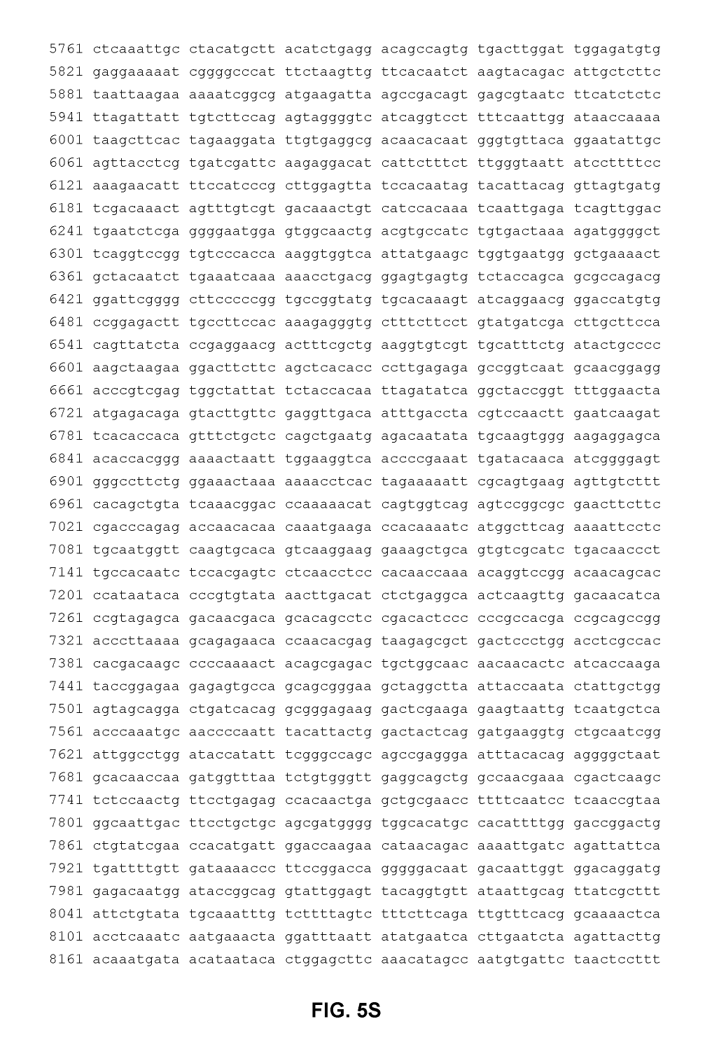

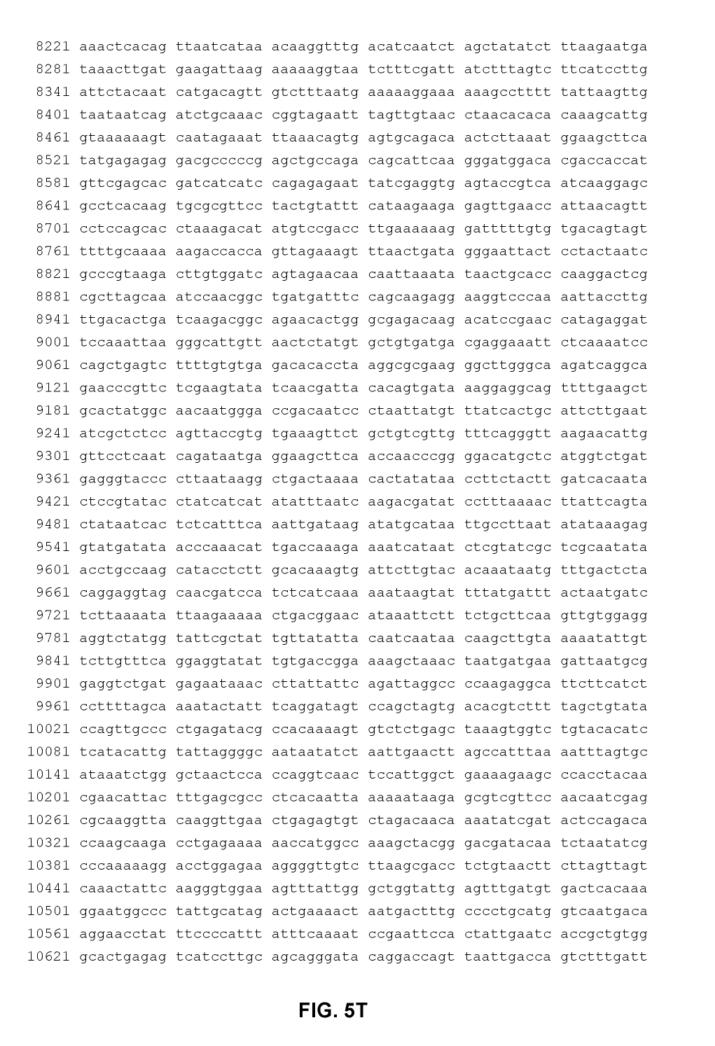

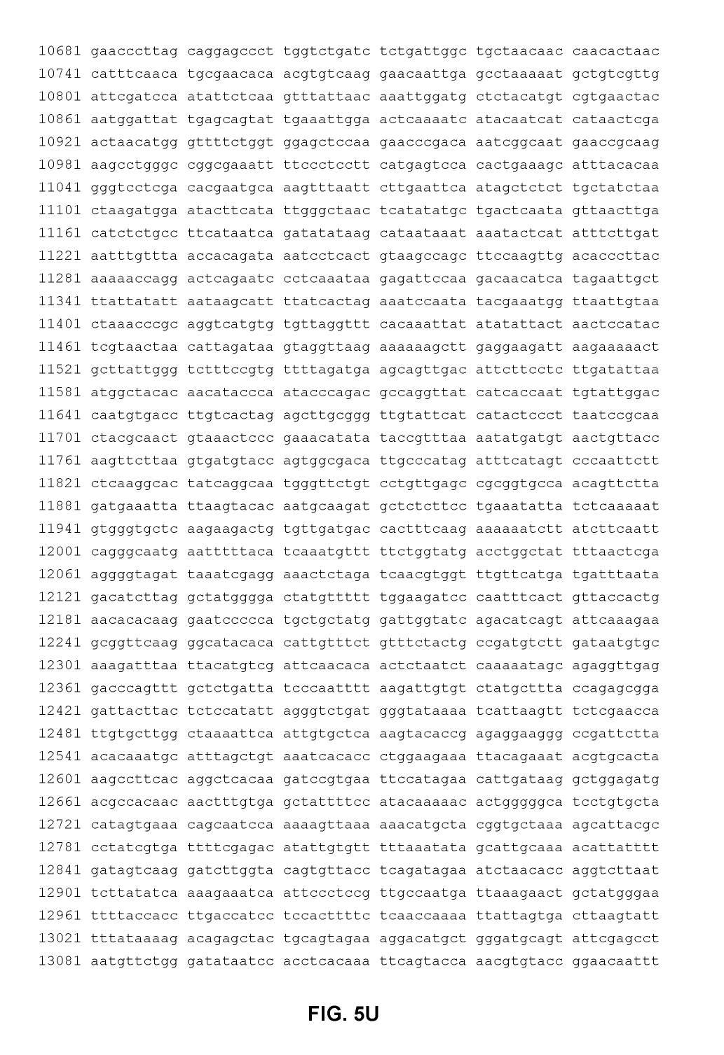

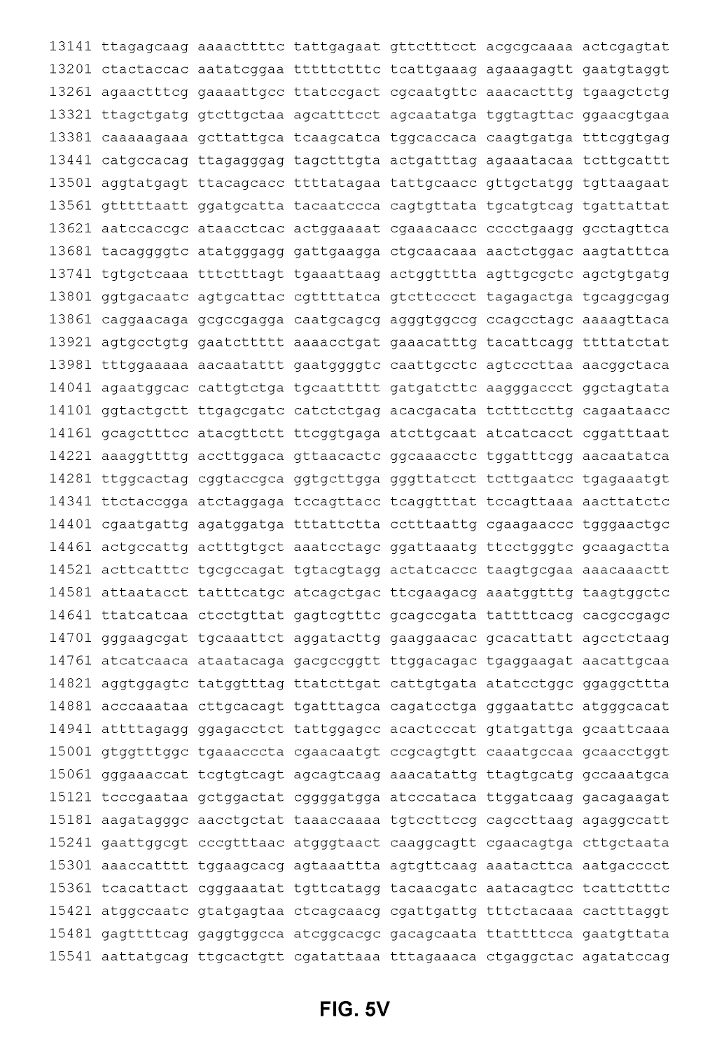

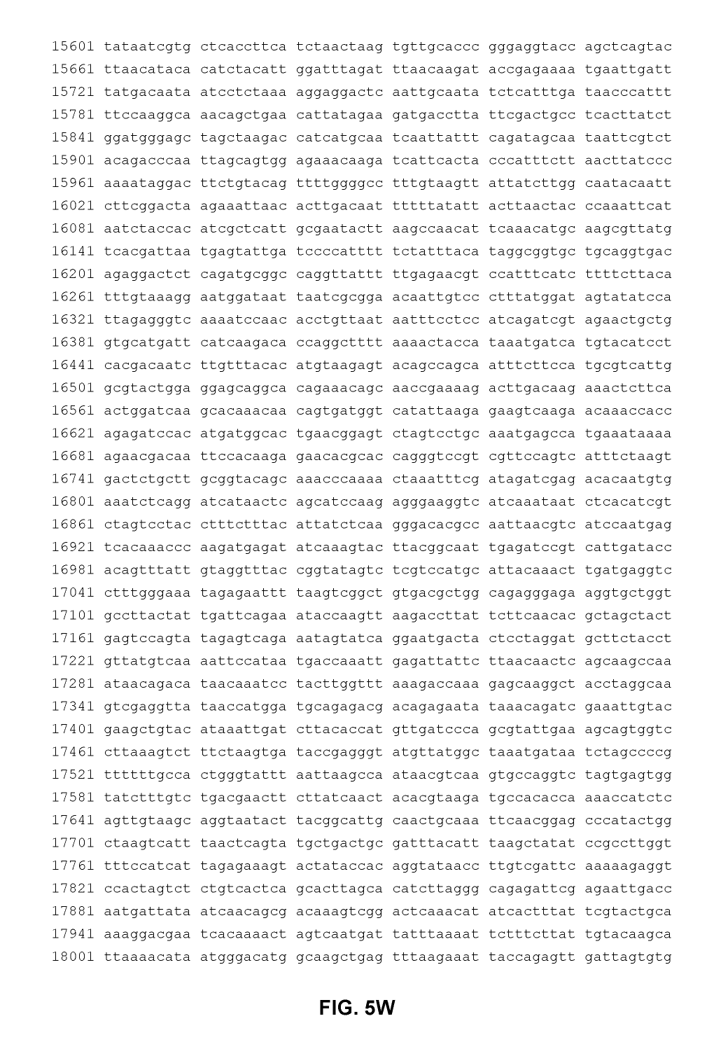

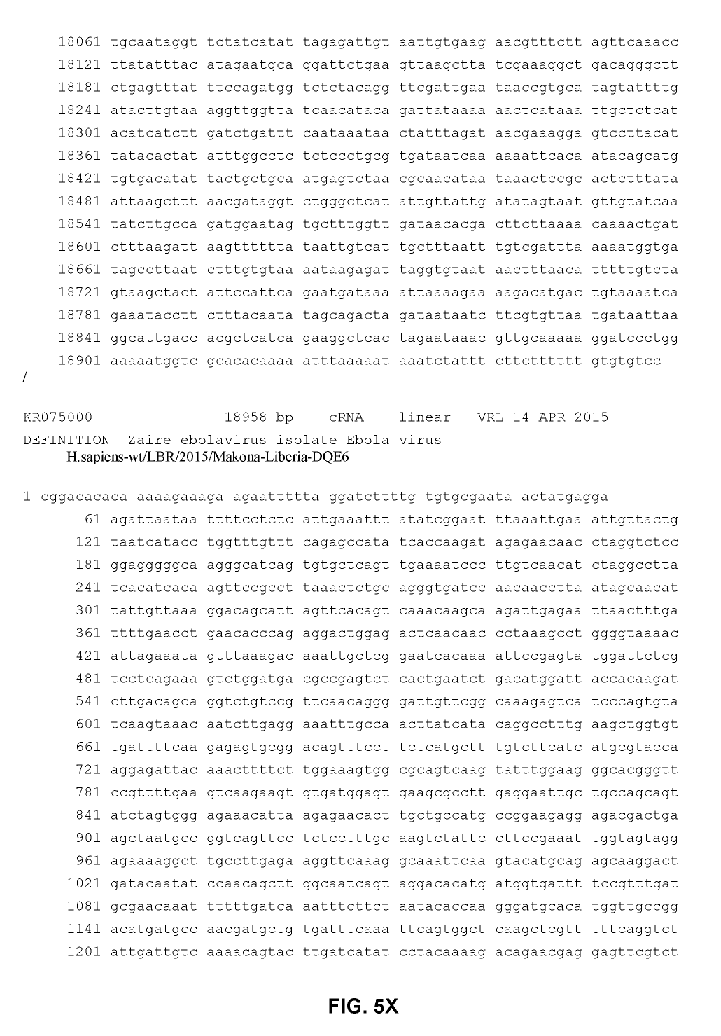

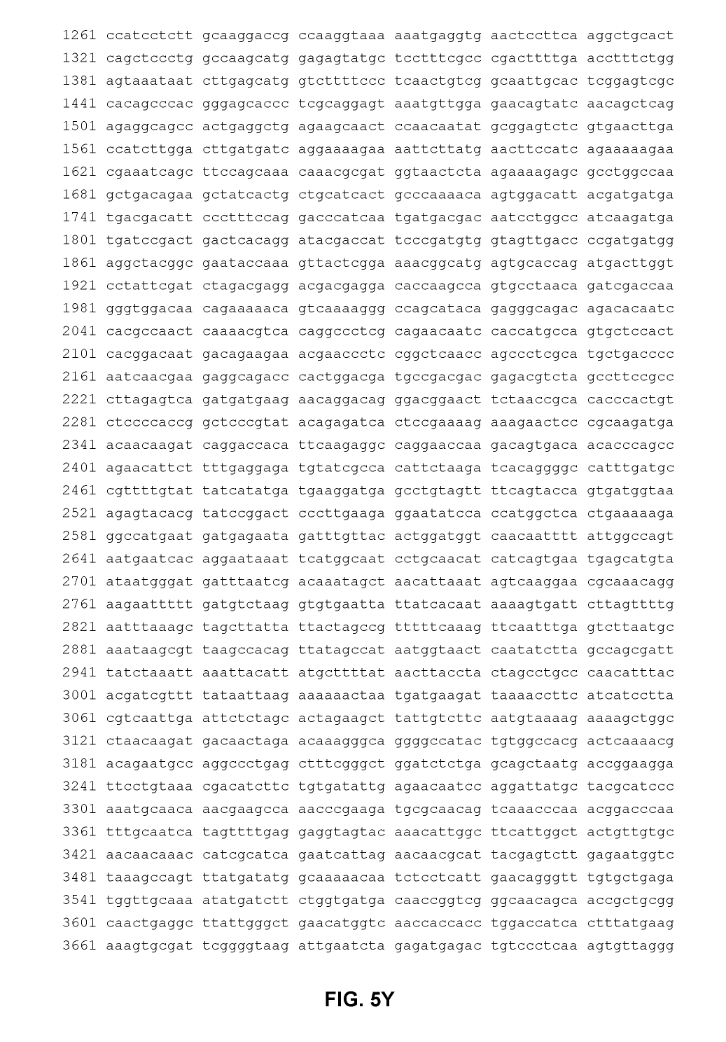

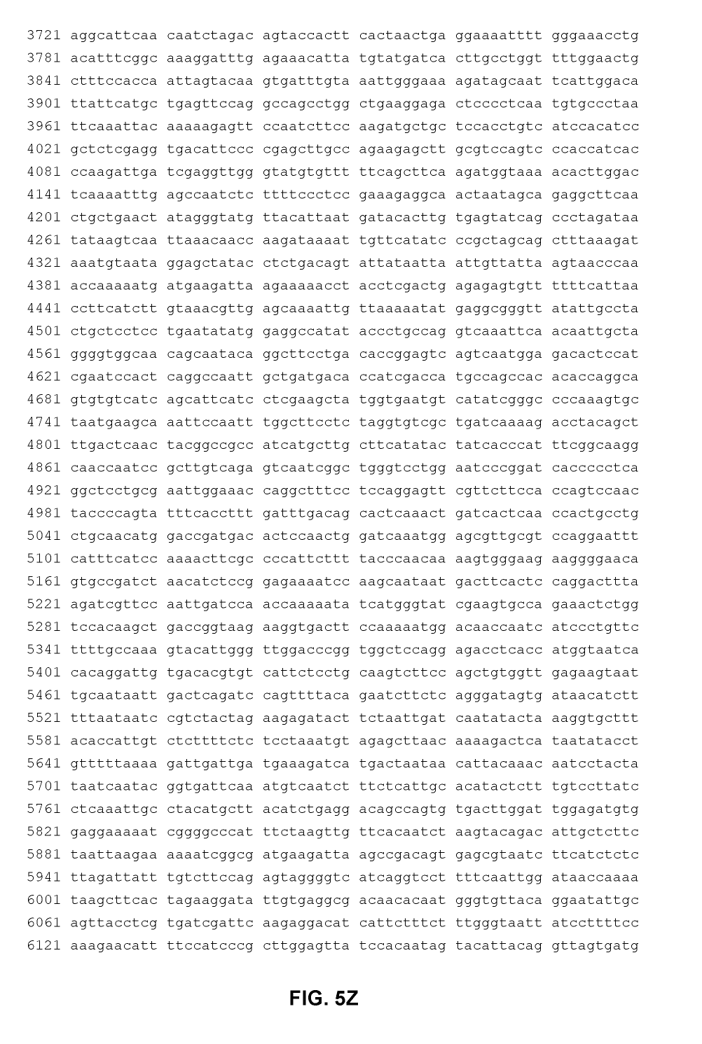

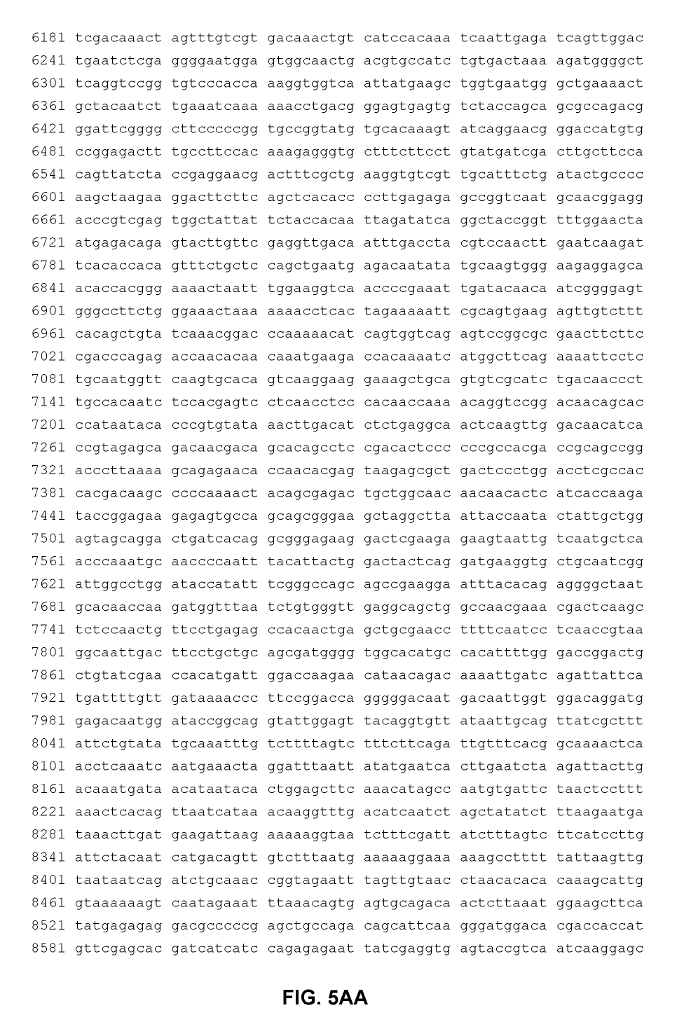

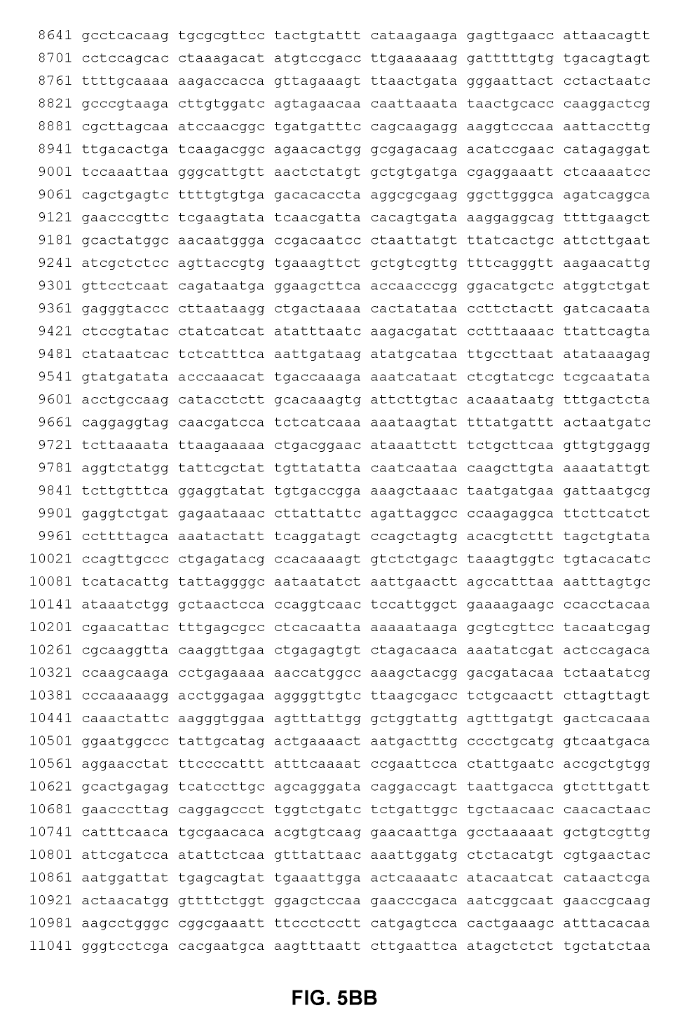

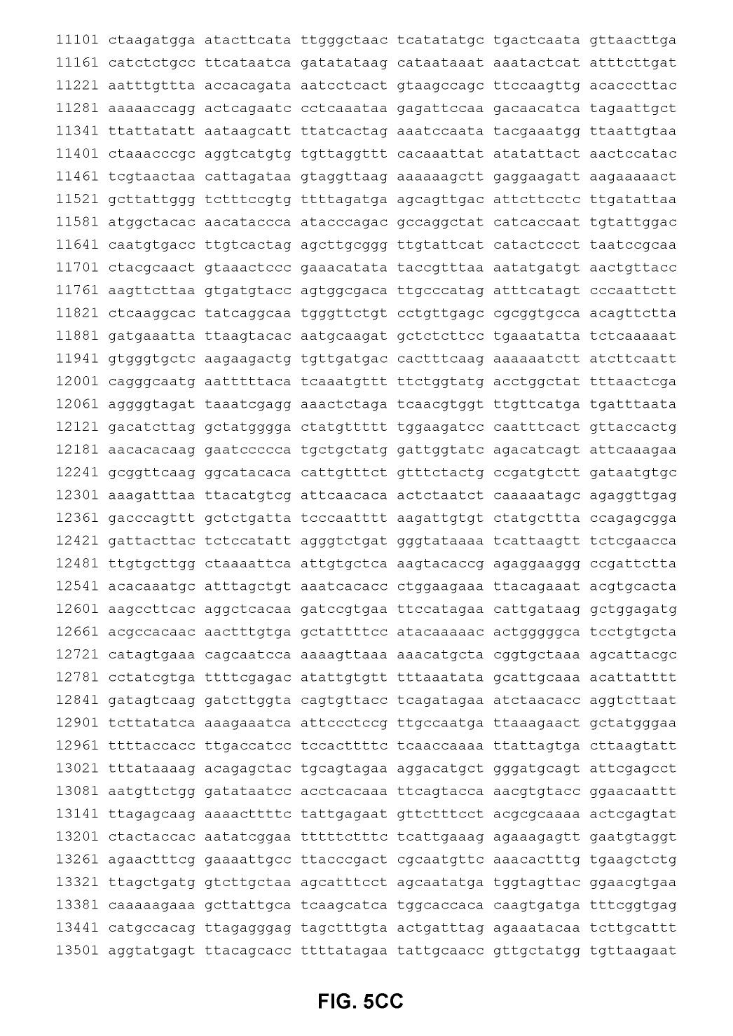

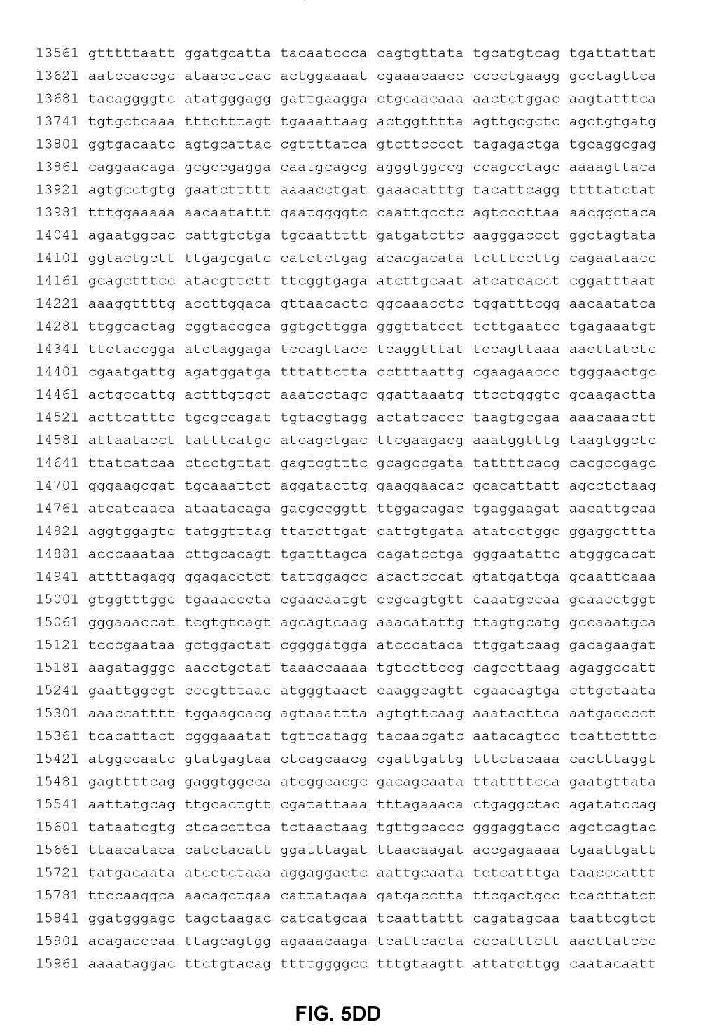

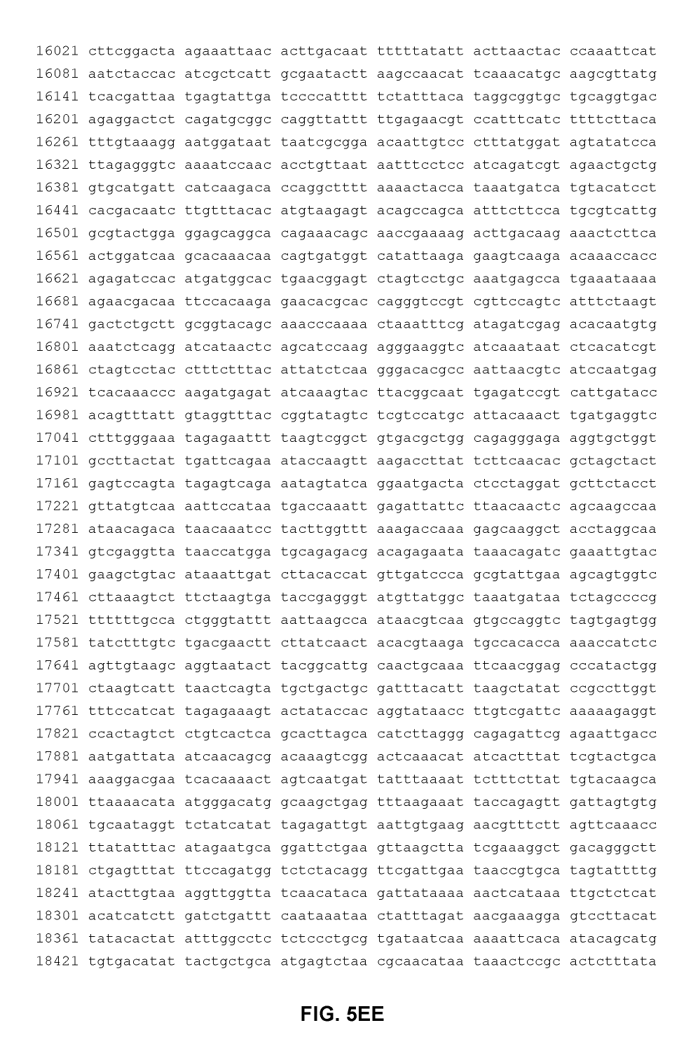

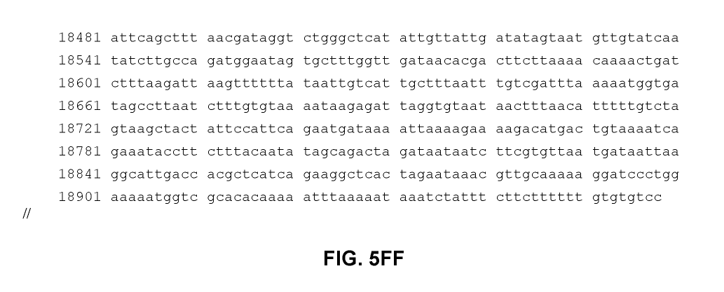

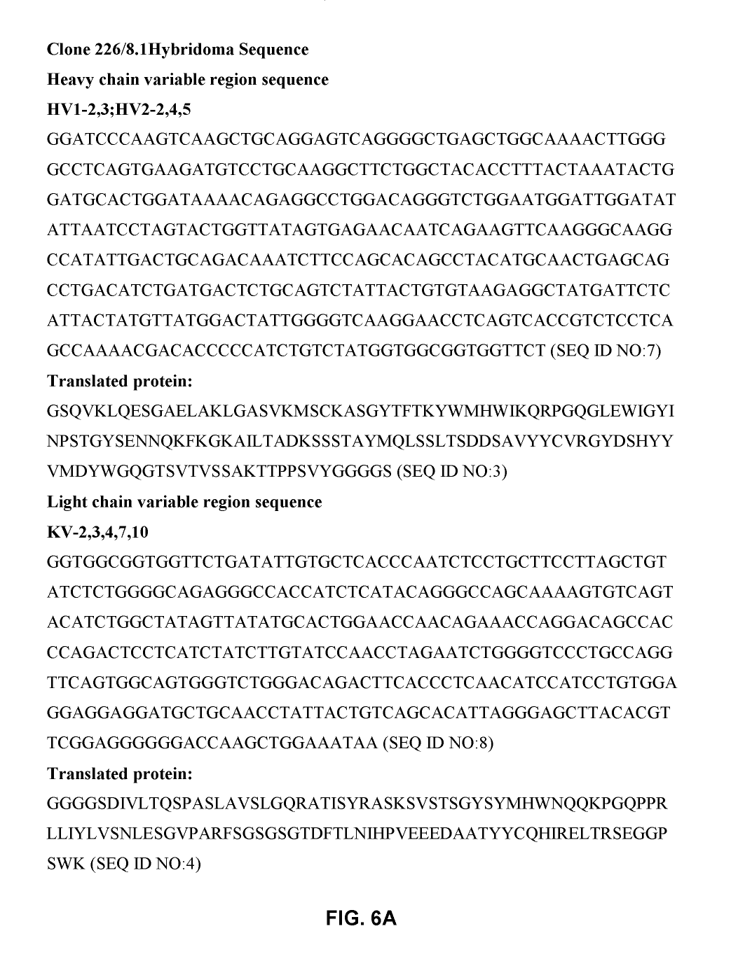

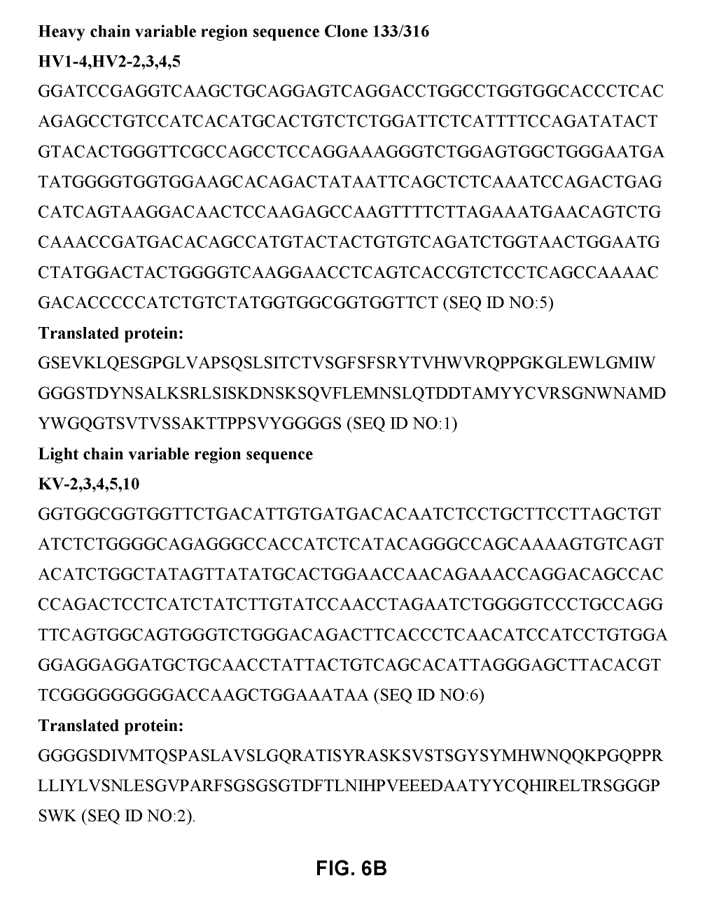

To identify GP regions involved in neutralization by these MAbs, a series of chimeric proteins was generated with GPs from the Zaire and Reston species (FIG. 4). MAb 133/3.16 neutralized the infectivity of VSV pseudotyped with RBbZGP (Reston, positions 1 to 415; Zaire, positions 418 to 676), RXZGP (Reston, positions 1 to 304; Zaire, positions 304 to 676), REZGP (Reston, positions 1 to 236; Zaire, positions 236 to 676), ZNRGP (Zaire, positions 1 to 560; Reston, positions 562 to 677), and RBsZNRGP (Reston, positions 1 to 461 and 562 to 677; Zaire, positions 461 to 560) hut not others, suggesting that this antibody recognizes a region in amino acid positions 521 to 560 of Zaire GP, By contrast, MAb 226/8.1 bound to a different region (positions 1 to 232) of Zaire GP, as indicated by the neutralization of infectivity of the virus with ZERGP (Zaire, positions 1 h/232; Reston, positions 234 to 677) or ZNRGP (Zaire, positions 1 to 560; Reston, positions 562 to 677), but not those with the other chimeric GPs. Since all three previously identified neutralizing epitopes are located in the region of amino acid positions 389 to 493 (Wilson et al., 2000), these results suggested the existence of two other neutralizing epitopes on GP.

Identification of Neutralizing Epitopes with a Recombinant VSV Containing the Ebola Virus GP-Encoding Gene.

To conclusively determine the neutralizing epitopes for these antibodies, antigenic variants were sought that escape from neutralization by the antibodies. A recombinant VSV containing the Ebola virus GP-encoding gene instead of the VSV G protein-encoding gene (chimeric VSV-EbolaGP) was generated. This virus expresses Ebola virus GP in the context of the VSV genome, utilizes Ebola virus GP for entry into cells, and grows rapidly in cell culture (107 to 108 PFU/ml in 2 to 3 days), as is the case with wild-type VSV, Thus, this VSV-EbolaGP chimera is useful for rapid selection of antigenic variants from GP-encoding gene pools in the VSV genome.

Chimeric VSV-EbolaGP was grown in the presence of either MAb 133/3.16 or 226/8.1, and antigenic variants that escaped from neutralization were isolated 3 days after infection. Three variants for each antibody were biologically cloned as described in Materials and Methods. The frequencies of isolation of the antigenic variants from the parent virus were 10^{-5.25} and 10^{-4.75} with MAbs 133/16.3 and 226/8.1, respectively. Sequence analysis of these variants' GPs revealed that each variant had a single amino acid change in the GP. All three variants selected with MAb 133/3.16 had the same His-to-Arg substitution at position 549 in GP2, which is adjacent to the fusion domain (10 amino acids downstream) of GP2. By contrast, MAb 226/8.1 selected three variants with different amino acid substitutions: Lou at position 199, Phe at position 194, or Arg at position 134 in GP1 was replaced with Ser. Ser, or Gln, respectively, suggesting that MAb 226/8.1 recognized a conformational epitope on the GP molecule. Consistent with this finding, this antibody did not react to GP in an immunoblot assay (data not shown). All amino acid substitutions were located in the regions predicted by the use of VSV pseudotyped with chimeric proteins. However, neither antibody bound to synthetic peptides containing the GP regions identified by the neutralization assay.

### Discussion

To identify B-cell epitopes for neutralization of Ebola virus, a recombinant VSV was used that contained the Ebola virus GP-encoding gene in place of the VSV G protein-encoding gene. This chimeric virus utilizes Ebola virus GP for cell entry, relying on VSV genes and proteins for replication and transcription of its genome and for viral protein synthesis. It therefore grows rapidly in cell culture, comparably to wild-type VSV. Consequently, this virus can be used to select GP antigenic variants more efficiently than wild-type Ebola virus, which does not grow in cultured cells as rapidly as VSV (taking a week to develop complete cytopathic effects). Hence, this chimeric VSV system should be useful for selecting antigenic variants from glycoproteins of viruses incapable of being cultured satisfactorily in vitro.

Since GP and sGP share approximately 300 N-terminal amino acids, they possess several epitopes in common (Sanchez et al., 2001; Volchkov et al., 1995). In fact, sGP adsorbs neutralizing antibodies in anti-Zaire GP serum (Ito et al., 2001). Since sGP is detected at a high concentration in the blood of acutely infected patients (Sanchez et al., 2001; Sanchez et al., 1996), neutralizing antibodies that do not react to sGP would be more effective for treatment of Ebola virus infection than those reacting to this molecule. In accord with this concept, neutralizing antibodies reacting with GP but not with sGP were reported to protect mice from lethal Ebola virus infection (Wilson et al., 2000). Single amino acid residues were identified in two other neutralizing epitopes, and neither of the antibodies used in this study bound to sGP in an ELISA (data not shown). Interestingly. MAb 226/8.1 did not bind to sGP even though Lou at position 199. Phe at position 194, and Arg at position 134 are shared by GP and sGP. Since Ebola virus GP and sGP are composed of trimers of GP1-GP2 and antiparallel-orientated homodimers, respectively (Sanchez et al., 1998; Volchkova et al., 1998), different oligomerization forms likely affect the tertiary structure of the conformational epitope. It is also conceivable that this epitope is not present on sGP monomers or may reside inside sGP dimers.

Neither of the MAbs used in this study neutralized the infectivity of VSV pseudotyped with GPs from the Sudan, Ivory Coast, and Reston species. It seems that there are few cross-neutralizing epitopes among Ebola virus species (Takada and Kawaoka, 2001). This antigenic difference must be considered for both passive prophylaxis and vaccination for Ebola virus infection. The use of neutralizing antibody cocktails, ideally cross-reactive among different Ebola virus species, may increase the protective effects of the treatments and reduce the possibility of the emergence of antigenic variants in the infected individuals.

### REFERENCES

Barbas et al., Proc. Natl. Acad. Sci. 88:7978 (1991).
Berzofsky, Science. 229:932 (1990).
Better et al., Science, 240:1041 (1988).
Bowie et al., Science, 247:1306 (1990),
Bray et al., J. Infect. Dis., 178:651 (1998).
Burton et al., Science, 266:1024 (1994).
Chothia and Lesk, J. Mol. Biol., 196901 (1987).
Clackson et al., Nature, 352:624 (1991),
Clark, The Experimental Foundations of Modern Immunology Wiley & Sons, Inc., New York (1986).
Duan et al., Proc. Natl. Acad. Sci, U.S.A., 91:5075 (1994).
Feldmann et al., Curr. Top. Microbiol. Immunol., 235:1 (1999).
Fischer and Emans, Transgenic Res., 9:279 (2000).
Giddings et al., Nat Biotechnol., 18:1151 (2000).
Gupta et al., J. Virol., 75:4649 (2001).
Hamers-Casterman et al., Nature, 363:446 (1993),
Harlow et al., Antibodies: A Laboratory Manual, (Cold Spring Harbor Laboratory Press, 2nd ed. (1988).
Herlyn et al., Science, 232:100 (1986).
Hiatt et al., Nature, 342:76 (1989).
Hodgson et al., Bio/Technology, 9:421 (1991).
Huse et al., Science, 246:1275 (1989).
Ito et al., J. Virol., 75:1576 (2001).
Jahrling et al., Arch. Virol. Suppl., 11:135 (1996).
Jahrling et al., J. Infect. Dis., 179:S224 (1999).
Jasenosky et al., J. Virol., 75:5205 (2001).
Jones et al., Nature, 321:522 (1986).
Kabat et al., U.S. Department of Health and Human Services, (1983).
Kang et al., Methods: A Companion to Methods in Enzymology, vol. 2, (1991).
Khaw et al., J. Nucl. Med., 23:1011 (1982).
Kohler and Milstein, Nature, 256:495 (1975).
Kudoyarova-Zubavichene et al., J. Infect. Dis. 79:S218 (1999).
Lerner and Burton, Academic Press, NY, pp 111
Levine et al., Meth, Enzymol., 11:928 (1967).
Lewis et al., Biochem., 22:948 (1983).
Marasco et al., Proc. Natl. Acad. Sci, U.S.A., 90:7889 (1993),
Marks et al., J Mol Biol., 222:581 (1991).
Mullinax et al., Proc, Natl. Acad. Sci, U.S.A., 87:8095 (1990).
Mupapa et al., J. Infect. Dis., 179:S18 (1999),
Pearson, Methods in Enzymology, 183:63 (1990).
Peters and Khan, Curr. Top. Microbiol, Immunol., 235:85 (1999).
Queen et al., Proc. Natl. Acad. Sci. USA, 86:10029 (1989).
Riechmann et al., Nature, 332:323 (1988).
Roberts et al., Proc. Natl. Acad. Sci. U.S.A., 89:2429 (1992).
Roitt, Essential Immunology, 7th Ed., (1991).
Rousseaux et al., Methods Enzymology, 121:663 (1986).
Sanchez et al., In D. M. Knipe and P. M. Howley (ed.), Fields virology, 4th ed, Lippincott Williams & Wilkins, Philadelphia, Pa., p. 1279 (2001).
Sanchez et al., J. Virol., 72:6442 (1998).
Sanchez et al., Proc. Natl. Acad. Sci. USA, 93:3602 (1996).
Sastry et al., Proc. Natl. Acad. Sc i. U.S.A., 86:5728 (1989).
Schnell et al., Proc. Natl. Acad. Sci. USA, 93:11359 (1996).
Shine and Dalgarno. Nature, 254:34 (1975), Smith and Waterman, Advances in Applied Mathematics, 2:482 (1981).
Smith et al., Science, 228:1315 (1985).
Takada and Kawaoka, Trends Microbiol., 9:506 (2001).
Takada et al., J. Virol., 75:2324 (2001).
Takada et al., Proc. Natl. Acad. Sci. USA, 94:14764 (1997).
Tempest et al., Biotechnology. 9:266 (1991).
Verhoeyen et al., Science, 239:1534 (1988).
Volchkov et al., Proc. Natl. Acad. Sci. USA, 95:5762 (1998).
Volchkov et al., Virology, 214:421 (1995).
Volchkova et al., Virology, 250:408 (1998).
Ward et al., Nature, 341:544 (1989).
Wasserman et al., J. Immunol., 87:290 (1961).
Whittle et al., Protein Eng., 1:499 (1987).
Wilson et al., Science, 287:1664 (2000).
Wool-Lewis and Bates, J. Virol., 72:3155 (1998).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A vector having a nucleic acid sequence encoding a polypeptide having SEQ ID NO:1, a polypeptide having SEQ ID NO:2, a polypeptide having SEQ ID NO:3, a polypeptide having SEQ ID NO:4, or a polypeptide having at least 90% amino acid identity to SEQ ID NO:1, 2, 3 or 4, or an Ebola virus binding fragment of the polypeptide.

2. The vector of claim 1 wherein the polypeptide having SEQ ID NO:1 is encoded by any of SEQ ID NO:5-25, the polypeptide having SEQ ID NO:2 is encoded by any of SEQ ID NO:26-46, the polypeptide having SEQ ID NO:3 is encoded by any of SEQ ID NO:48-68, or the polypeptide having SEQ ID NO:4 is encoded by any of SEQ ID NO:69-89.

3. The vector of claim 1 further comprising a promoter.

4. The vector of claim 1 wherein the polypeptide has at least 95% amino acid identity to SEQ ID NO:1, SEQ ID NO:2, SEQ ID NO:3, or SEQ ID NO:4.

5. The vector of claim 1 wherein the polypeptide is encoded by a nucleic acid sequence having at least 80% nucleic acid sequence identity to a nucleic acid encoding SEQ ID NO:1, SEQ ID NO:2, SEQ ID NO:3, or SEQ ID NO:4.

6. The vector of claim 1 wherein the polypeptide encodes an Ig heavy chain or an Ig light chain, or a ScFv.

7. The vector of claim 6 which encodes an IgG heavy chain.

8. An isolated host cell having the vector of claim 1.

9. The host cell of claim 8 which has a vector having a nucleic acid sequence encoding a polypeptide having at least 90% amino acid identity to SEQ ID NO:1, or an Ebola virus binding fragment of the polypeptide, and a polypeptide having at least 90% amino acid identity to SEQ ID NO:2, or an Ebola virus binding fragment of the polypeptide, or which has a vector having a nucleic acid sequence encoding a polypeptide having at least 90% amino acid identity to SEQ ID NO:1, or an Ebola virus binding fragment of the polypeptide, and a vector having a nucleic acid sequence encoding a polypeptide having at least 90% amino acid identity to SEQ ID NO:2, or an Ebola virus binding fragment of the polypeptide.

10. The host cell of claim 8 which has a vector having a nucleic acid sequence encoding a polypeptide having at least 90% amino acid identity to SEQ ID NO:3, or an Ebola virus binding fragment of the polypeptide, and a polypeptide having at least 90% amino acid identity to SEQ ID NO:4, or an Ebola virus binding fragment of the polypeptide or which has a vector having a nucleic acid sequence encoding a polypeptide having at least 90% amino acid identity to SEQ ID NO:3, or an Ebola virus binding fragment of the polypeptide, and a vector encoding a nucleic acid sequence encoding a polypeptide having at least 90% amino acid identity to SEQ ID NO:4, or an Ebola virus binding fragment of the polypeptide

11. The host cell of claim 8 which is a mammalian cell.

12. The host cell of claim 8 which is an insect, bacterial or yeast cell.

13. The host cell of claim 8 which is a plant cell.

14. The host cell of claim 13 wherein the plant cell is a dicot cell.

15. The host cell of claim 13 wherein the plant cell is a monocot cell.

16. The host cell of claim 13 wherein the plant cell is a tobacco cell, an alfalfa cell, a maize cell, a soybean cell, a rice cell or an Arabidopsis cell.

17. A method to prevent, inhibit or treat Ebola virus infection, comprising administering to a mammal an effective amount of a composition having an antibody comprising a polypeptide comprising SEQ ID NO:1 and a polypeptide comprising SEQ ID NO:2, or an isolated polypeptide comprising SEQ ID NO:1 and/or SEQ ID NO:2, or a polypeptide having at least 95% amino acid identity thereto, or an Ebola virus binding portion thereof, or a composition comprising an antibody comprising a polypeptide comprising SEQ ID NO:3 and a polypeptide comprising SEQ ID NO:4, or an isolated polypeptide comprising SEQ ID NO:3 and/or SEQ ID NO:4, or a polypeptide having at least 95% amino acid identity thereto, or an Ebola virus binding fragment thereof.

18. An isolated antibody comprising a polypeptide comprising at least 90% but not 100% amino acid sequence identity to SEQ ID NO:1, and optionally comprising a polypeptide comprising at least 90% amino acid sequence identity to SEQ ID NO:2; or an isolated antibody comprising a polypeptide comprising at least 90% but not 100% amino acid sequence identity to SEQ ID NO:2, and optionally comprising a polypeptide comprising at least 90% amino acid sequence identity to SEQ ID NO:1; or an isolated antibody comprising a polypeptide comprising at least 90% but not 100% amino acid sequence identity to SEQ ID NO:3, and optionally comprising a polypeptide comprising at least 90% amino acid sequence identity to SEQ ID NO:4; or an isolated antibody comprising a polypeptide comprising at least 90% but not 100% amino acid sequence identity to SEQ ID NO:4, and optionally comprising a polypeptide comprising at least 90% amino acid sequence identity to SEQ ID NO:3; or a hybridoma secreting an antibody comprising a polypeptide comprising at least 90% amino acid sequence identity to SEQ ID NO:1, and optionally comprising a polypeptide comprising at least 90% amino acid sequence identity to SEQ ID NO:2; or an isolated antibody comprising a polypeptide comprising at least 90% amino acid sequence identity to SEQ ID NO:2, and optionally comprising a polypeptide comprising at least 90% amino acid sequence identity to SEQ ID NO:1; or an isolated antibody comprising a polypeptide comprising at least 90% amino acid sequence identity to SEQ ID NO:3, and optionally comprising a polypeptide comprising at least 90% amino acid sequence identity to SEQ ID NO:4; or an isolated antibody comprising a polypeptide comprising at least 90% amino acid sequence identity to SEQ ID NO:4, and optionally comprising a polypeptide comprising at least 90% amino acid sequence identity to SEQ ID NO:3.


SEQUENCE LISTING (97 sequences)

SEQ ID NO: 1 - protein, 133 aa, Mus musculus
GSEVKLQESGPGLVAPSQSLSITCTVSGFSFSRYTVHWVRQPPGKGLEWLGMIWGGGSTDYNSALKSRLSISKDNSKSQVFLEMNSLQTDDTAMYYCVRSGNWNAMDYWGQGTSVTVSSAKTTPPSVYGGGGS

SEQ ID NO: 2 - protein, 113 aa, Mus musculus
GGGGSDIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSGGGPSWK

SEQ ID NO: 3 - protein, 136 aa, Mus musculus
GSQVKLQESGAELAKLGASVKMSCKASGYTFTKYWMHWIKQRPGQGLEWIGYINPSTGYSENNQKFKGKAILTADKSSSTAYMQLSSLTSDDSAVYYCVRGYDSHYYVMDYWGQGTSVTVSSAKTTPPSVYGGGGS

SEQ ID NO: 4 - protein, 113 aa, Mus musculus
GGGGSDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 5 - DNA, 399 bp, Mus musculus
ggatccgaggtcaagctgcaggagtcaggacctggcctggtggcaccctcacagagcctgtccatcacatgcactgtctctggattctcattttccagatatactgtacactgggttcgccagcctccaggaaagggtctggagtggctgggaatgatatggggtggtggaagcacagactataattcagctctcaaatccagactgagcatcagtaaggacaactccaagagccaagttttcttagaaatgaacagtctgcaaaccgatgacacagccatgtactactgtgtcagatctggtaactggaatgctatggactactggggtcaaggaacctcagtcaccgtctcctcagccaaaacgacacccccatctgtctatggtggcggtggttct

SEQ ID NO: 6 - DNA, 342 bp, Mus musculus
ggtggcggtggttctgacattgtgatgacacaatctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcgggggggggaccaagctggaaataa

SEQ ID NO: 7 - DNA, 408 bp, Mus musculus
ggatcccaagtcaagctgcaggagtcaggggctgagctggcaaaacttggggcctcagtgaagatgtcctgcaaggcttctggctacacctttactaaatactggatgcactggataaaacagaggcctggacagggtctggaatggattggatatattaatcctagtactggttatagtgagaacaatcagaagttcaagggcaaggccatattgactgcagacaaatcttccagcacagcctacatgcaactgagcagcctgacatctgatgactctgcagtctattactgtgtaagaggctatgattctcattactatgttatggactattggggtcaaggaacctcagtcaccgtctcctcagccaaaacgacacccccatctgtctatggtggcggtggttct

SEQ ID NO: 8 - DNA, 342 bp, Mus musculus
ggtggcggtggttctgatattgtgctcacccaatctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaataa

SEQ ID NO: 9 - DNA, 399 bp, Artificial Sequence
  A synthetic oligonucleotide
gggtcagaggtgaagctccaggagtccgggcccggcctggtggcaccttctcagagtctcagtatcacctgcacagtaagcgggttttctttctcccgatatactgtgcactgggtcagacagccccccggaaaaggcctggagtggctgggcatgatctggggaggtggcagcacggactacaattccgctctcaaaagccgcctcagtatctccaaagataatagcaaatcccaagttttccttgaaatgaattctcttcaaacagacgacaccgccatgtactactgtgtgcgatctgggaattggaacgctatggattactggggtcaggggaccagtgtaacagtatcttctgcgaagactactcctccaagcgtctacggaggtggaggaagc

SEQ ID NO: 10 - DNA, 399 bp, Artificial Sequence
  A synthetic oligonucleotide
ggctctgaggtgaagctgcaggagtccggaccaggccttgtcgcccccagccagagcctgagcatcacttgtaccgtcagcgggttctcctttagtcggtatacagtccattgggtgagacagccccccgggaagggattggagtggcttggtatgatatgggggggaggaagcactgattataattctgcccttaaatccaggctgagcatttcaaaagacaacagcaagagccaggtctttctcgaaatgaactcactgcaaacggacgacaccgctatgtattactgcgtgcgcagtggcaactggaacgctatggattattgggggcagggaacatccgtcacggtatcatctgccaagaccacacccccctcagtgtatggcgggggcggttcc

SEQ ID NO: 11 - DNA, 399 bp, Artificial Sequence
  A synthetic oligonucleotide
ggtagtgaagtgaagctgcaggagagcggacctggcctggtcgcgcccagtcagtccctgtcaatcacttgcaccgtttccggcttttccttctccagatataccgtgcactgggtacgccaaccaccggggaaggggctggaatggcttggcatgatctggggcggagggtctactgactacaactcagccctgaagagtagactttccatctccaaagataactccaagagccaggtgttcttggagatgaattccctccagaccgacgatacagccatgtactactgcgtgcggtctggaaattggaacgcaatggattactggggacagggaacatccgtcacagtcagtagcgctaagacaaccccacccagtgtgtacggtggtgggggaagc

SEQ ID NO: 12 - DNA, 399 bp, Artificial Sequence
  A synthetic oligonucleotide
ggcagcgaagtgaaactccaggaaagcggccctggcctcgtcgctccgtcacagagcctttcaattacttgcactgtgagcggatttagttttagtcgctacacagtgcattgggttagacagcctccaggtaagggcctcgagtggctgggcatgatttggggcgggggctcaaccgattacaatagtgccctgaagtccagactctcaattagtaaggacaatagcaaatctcaggtgttcctggagatgaattcccttcagactgacgacacagcaatgtactactgcgtgcggagcgggaactggaatgccatggactactggggccaaggcactagcgtgacggtaagctcagcaaagactactcccccttctgtttacggaggtggtggaagc

SEQ ID NO: 13 - DNA, 399 bp, Artificial Sequence
  A synthetic oligonucleotide
ggatcagaggtgaaactgcaagagtctgggccaggtcttgtggctccgtcacagtccctgtcaatcacttgtactgtctctggattcagcttttctagatacaccgtccactgggtgcggcagcccccgggcaaggggctcgagtggctgggaatgatctggggagggggctccactgactacaactccgctttgaaaagtcggctctcaatctccaaagacaactccaagtctcaagtgttcctcgaaatgaactcccttcagaccgatgatacagcgatgtattactgcgtgaggtcagggaactggaacgcaatggattattggggtcaaggcacctcagttacggtttccagcgctaaaactaccccccccagcgtgtatggcggcggcggatct

SEQ ID NO: 14 - DNA, 399 bp, Artificial Sequence
  A synthetic oligonucleotide
ggcagcgaagtaaagttgcaagaatcaggacctggacttgtagctccgagccagtccctgtccattacatgcacagtttctggcttctctttcagccgatataccgtgcactgggttaggcagccccccggtaaagggctggaatggctggggatgatttggggcggagggagtaccgactataactccgctctgaaatcaaggctcagcatatccaaggacaacagcaagagtcaggtgttcttggaaatgaatagccttcagacggacgacactgccatgtactattgcgtcagaagcgggaactggaatgcgatggattactggggccagggaacttccgttacagtgtctagtgcgaaaactacgccccccagcgtgtacggcggcggcgggtct

SEQ ID NO: 15 - DNA, 399 bp, Artificial Sequence
  A synthetic oligonucleotide
gggtccgaggtgaagttgcaagagtctggccccggtcttgtggctcctagtcaaagcctttctataacttgcacagtgtccggcttctcttttagccgatatacagtccattgggtgagacagcctcctggaaagggcctcgagtggctgggtatgatctgggggggcggcagtacagactataatagtgccctgaaatctcgcctctcaatcagcaaggataacagtaagagccaggtcttcctcgaaatgaacagcctccagacagatgatacggccatgtattattgcgtgcgcagcggaaactggaatgctatggactattgggggcaggggacatccgtgactgttagtagcgccaaaactacgccaccttcagtctatggtggcgggggttcc

SEQ ID NO: 16 - DNA, 399 bp, Artificial Sequence
  A synthetic oligonucleotide
ggttcagaagtcaagctgcaagagtctggtcccggtttggtagctcctagtcaatccctgtccattacctgtacagtttctggcttttctttttctagatatacagttcattgggtccgtcagcctcccggaaagggattggagtggcttggtatgatatggggtggtgggtctacagattacaattctgcactaaagagccgtctttctatttctaaggataacagtaagagccaggttttccttgaaatgaactcattacaaactgatgatacggcaatgtattattgtgttcgttctggtaactggaatgccatggattattggggacaaggaacttccgtgactgtttctagtgctaagaccactccaccttctgtctatgggggaggcggttca

SEQ ID NO: 17 - DNA, 399 bp, Artificial Sequence
  A synthetic oligonucleotide
ggttcagaggtgaagctgcaagaaagtggacctggtctggtagccccgtctcaaagcctttctatcacctgtactgtctcagggttttcattttccagatacacagttcattgggtccgacagcctcctgggaaaggtttggagtggttgggtatgatttgggggggaggatcaactgattataattctgctctcaagtccagactctcaatatcaaaggacaactcaaagagccaagtatttcttgaaatgaattctcttcaaaccgacgatactgcaatgtattattgtgtaaggagcggaaattggaatgctatggattactggggacagggaacgtctgttaccgtatcttcagcaaaaactactccaccaagtgtttatggagggggaggatct

SEQ ID NO: 18 - DNA, 399 bp, Artificial Sequence
  A synthetic oligonucleotide
ggcagtgaggttaagttgcaagagtccggtccaggactcgtagccccttctcagtctctgtccattacttgtacagtttctggattttctttctctagatatactgtacactgggtgaggcagcctcctggtaaaggtctcgagtggctgggtatgatatggggagggggtagcaccgactataatagcgcactcaagagtaggttgtccatatctaaggataattctaagtcccaggtgtttctagaaatgaatagcctgcagacagatgacacagcaatgtactattgcgttcggtctggcaattggaatgctatggactactggggtcagggaacgagtgtgactgtttcctctgcaaagacaacgccgcctagtgtttacggtggtggaggctcc

SEQ ID NO: 19 - DNA, 399 bp, Artificial Sequence
  A synthetic oligonucleotide
ggaagcgaagtaaagttgcaggaaagtgggcctggacttgtggcaccttctcaatctttgagcatcacatgtacagtaagcggattctcttttagtcgatataccgtgcattgggttagacaacctccagggaaaggcctcgagtggttgggaatgatctggggcggtggtagtactgattacaactctgctcttaaatcacgactgtctatatccaaggacaattcaaaatcccaggttttcttagagatgaattctttacagacagacgataccgcaatgtattactgcgttcgtagcggcaactggaatgccatggattattggggtcaggggacttccgttacagtgagtagtgccaaaacgacaccacccagtgtttatggtggaggtgggtca

SEQ ID NO: 20 - DNA, 399 bp, Artificial Sequence
  A synthetic oligonucleotide
ggttcagaggttaagttgcaggaatctggaccaggactagtggccccctctcagtctctaagcattacctgtactgtctccggtttcagtttctctaggtacactgtccattgggttaggcagccacctggtaaaggtttggaatggttgggtatgatttggggtggaggatcaacggattataacagtgcactgaagtcccgtttgtctataagtaaggataactcaaaatctcaagttttcttggaaatgaattctctccaaacagatgatacggcaatgtactactgtgtgaggtcaggtaattggaatgccatggactactgggggcaaggaacctctgttaccgttagttccgcaaagacaacacctccatcagtatacggtggaggcggaagc

SEQ ID NO: 21 - DNA, 399 bp, Artificial Sequence
  A synthetic oligonucleotide
ggatctgaggttaaattgcaggaaagcggaccggggttagtggctcctagtcaatctttgtcaatcacatgtacagtctcaggtttttcattctctaggtatactgtgcattgggtcagacaaccaccgggtaaaggattggaatggcttggaatgatatggggtggaggtagtactgattacaacagcgctttgaaaagccggttatccatttctaaagataactctaaatcacaagtgtttttggagatgaattccctccagactgatgatacggcaatgtattactgcgtgagatcaggcaactggaacgcaatggactactggggacaaggaacttcagttactgtttcatctgctaagacaacacctccatccgtgtacggtggcgggggttca

SEQ ID NO: 22 - DNA, 399 bp, Artificial Sequence
  A synthetic oligonucleotide
ggcagtgaagtaaagctccaggaaagtggtcctggattagtagctcctagccaaagtctgtctattacctgcactgtttcaggcttcagtttttccaggtatacagttcattgggtgcgtcagcctccaggtaaggggctggaatggcttggtatgatctgggggggcgggtctacagactataactcagctcttaaatcacgtctctctatctctaaggataacagcaagtctcaagtatttcttgaaatgaacagcttgcaaacagatgataccgctatgtactactgtgtacgatctgggaattggaacgcaatggattattggggccaggggactagcgttacagtttctagtgctaagacaacaccaccatcagtttacggaggcggagggtcc

SEQ ID NO: 23 - DNA, 399 bp, Artificial Sequence
  A synthetic oligonucleotide
ggaagcgaagttaagcttcaggagagtgggccaggtttggtagcaccttctcagtctttgagtattacatgtacggtgtccggattctcattttctcgatatactgttcattgggttagacaaccacctggaaagggtttagagtggttgggtatgatttggggcggtggttccactgattacaactcagcactgaagagtaggttaagtataagtaaggataactctaaatcacaggtttttcttgaaatgaactctttacagactgatgatactgctatgtactactgcgtcagatctggaaactggaacgcaatggattattggggccagggaacttctgttactgttagctccgctaagaccacacccccgagtgtctacgggggtgggggaagc

SEQ ID NO: 24 - DNA, 399 bp, Artificial Sequence
  A synthetic oligonucleotide
ggcagtgaggttaagcttcaagaaagcggacccggcctcgttgctccatctcaatcactgtctattacctgtaccgtttccggattttcattctctagatatactgttcattgggtgcggcaaccacctgggaagggactcgaatggcttggtatgatatggggtggtggttcaacagattataacagtgctcttaagtctcgactctccatctccaaagataactcaaagagccaggttttcttggaaatgaatagccttcaaacagacgatacggctatgtattattgcgtacgttccgggaattggaatgcaatggactactggggtcaaggtacgtcagttacagtgtctagtgccaagaccacaccaccatctgtctatggtggaggtgggagt

SEQ ID NO: 25 - DNA, 399 bp, Artificial Sequence
  A synthetic oligonucleotide
gggagcgaggttaaattacaggagtctgggcctgggttagtggctccaagtcagagtctctctattacttgtactgtctctggattttctttttcaagatatactgttcattgggttcgtcaacctccagggaagggtctggagtggttgggaatgatctggggcggcggatcaacggattataattccgctttgaagtccagattatctattagcaaagataacagtaagtcccaggtttttttagaaatgaatagcttacaaaccgatgatacagctatgtattattgtgttagatcaggtaattggaatgctatggattactggggacagggtacaagtgttactgtctccagcgctaagactacaccaccaagtgtgtatggtggcgggggttca

SEQ ID NO: 26 - DNA, 342 bp, Mus musculus; identical to SEQ ID NO: 6

SEQ ID NO: 27 - DNA, 342 bp, Artificial Sequence
  A synthetic oligonucleotide
ggagggggcggatccgatatcgtaatgacgcagtcccctgcatctctggctgtgtccctcggacaaagggccactatctcttatagagctagcaagtctgtatcaacatccggatacagttacatgcactggaatcagcagaagcccggtcaaccgcctcgcctgctgatctacctggtgtccaacttggagtccggcgtgccagccagatttagtgggtccggttcagggaccgactttacacttaatattcacccagttgaagaggaagacgcagccacttactattgccagcacatcagggaactgacgcgaagcggcggcggtccgtcatggaagtga

SEQ ID NO: 28 - DNA, 342 bp, Artificial Sequence
  A synthetic oligonucleotide
gggggcggaggaagtgacattgtgatgacgcaaagtcctgcctccctggccgtgtctctgggacagagagcgacgatctcctacagggctagcaagtccgtttccacgtcaggatatagttacatgcactggaatcagcagaagcccggccagcccccaagattgttgatttacctcgtcagtaaccttgaatctggcgtgcccgcccggttcagtgggtctggttccggaacggatttcacactgaacattcaccctgttgaagaggaagatgccgccacatactactgtcagcatatccgggagctgacaaggagtggcggaggaccaagctggaagtaa

SEQ ID NO: 29 - DNA, 342 bp, Artificial Sequence
  A synthetic oligonucleotide
ggcggtggtggctccgatatcgtgatgacccagtccccagccagtcttgccgtttccctcggtcaacgagcaactatcagctaccgggcctcaaagagcgtctccacatctggatattcctacatgcactggaatcagcaaaagcctggccaaccaccccggctcctgatatacttggtatctaatctggaatcaggagtgcccgcaagattttctggtagtggctccggcacagacttcaccctcaacattcaccctgtggaggaagaggacgccgcaacttattattgtcagcatatccgcgaactgacgagatcagggggcggtccaagttggaagtga

SEQ ID NO: 30 - DNA, 342 bp, Artificial Sequence
  A synthetic oligonucleotide
ggagggggaggatctgacattgtgatgactcagtcccctgctagcctcgccgttagtctgggacagagagccaccatctcctatcgagctagcaagtccgtaagcacaagcgggtacagttatatgcactggaaccaacagaagccaggacagccgcccagactgctgatttatctggtgagtaacttggagtccggcgtgcctgccagattcagtggctcagggagtggcaccgacttcaccctcaatattcatcccgtcgaggaagaagatgcagcgacatactactgccagcacattagggagctgacccggagtggcggcgggccctcatggaaatga

SEQ ID NO: 31 - DNA, 342 bp, Artificial Sequence
  A synthetic oligonucleotide
ggtgggggcggaagcgacatcgtcatgacacagtctcctgccagtctggccgtgagcttgggccagcgagccacaatctcttacagagctagcaaatctgtgagcacgtcaggctattcatacatgcactggaaccagcaaaaacccgggcagccaccccgacttttgatatatctcgtgagtaacctggaatccggcgtgccggcgcggttttccggttccgggagtgggacagatttcacacttaacattcatcccgttgaagaagaggacgccgcgacgtactattgccagcacattcgggaacttacgcgatcaggtggcggtcccagctggaaataa

SEQ ID NO: 32 - DNA, 342 bp, Artificial Sequence
  A synthetic oligonucleotide
ggcggaggtggttccgatatagtgatgactcagtctcccgcctccctggctgtgtcactcggccaaagggctaccatttcctaccgcgctagcaaaagcgttagtacctctggctacagttatatgcattggaaccagcaaaagcctgggcagccgccccgattgcttatctacctcgttagcaacctcgagagtggggtgccagctcgcttctccgggtccgggtctggcaccgatttcaccctgaacattcaccctgtggaagaagaggacgcagcgacctattactgccagcatatccgcgaactgactcggagtggagggggaccatcttggaaataa

SEQ ID NO: 33 - DNA, 342 bp, Artificial Sequence
  A synthetic oligonucleotide
ggaggtggcgggagcgatatcgtgatgacccaatcccctgcctcccttgccgtgtcacttggccagagggccactatctcttaccgcgcctcaaagtctgtgtctacctctggatattcatatatgcactggaatcagcagaagcccggacagcccccgagattgctgatttatctggtgagcaaccttgagtctggagtgcccgccagattcagtggatctggcagcggaaccgattttacactgaatattcaccctgtggaggaagaagacgcagcaacatactattgccagcatatcagagagctcactcggtccggcggcggtccctcttggaaatga

SEQ ID NO: 34 - DNA, 342 bp, Artificial Sequence
  A synthetic oligonucleotide
gggggagggggcagcgatattgtcatgactcaatccccagccagtcttgccgtctcacttgggcagagagctactatcagctacagggccagcaagtccgtgagcacctccggatactcttatatgcactggaatcagcagaagcccggccagccaccaagactgttgatctacctcgttagcaatctggagtctggtgtccccgctcggttttcaggatccggatctgggacggattttactctcaacatccaccctgtagaggaggaggatgctgcaacctactactgccagcatatcagggagcttactagatcaggtggcggaccatcttggaagtga

SEQ ID NO: 35 - DNA, 342 bp, Artificial Sequence
  A synthetic oligonucleotide
ggaggcggtggctccgacatcgtcatgactcagagtcccgcatccctcgctgtctcactcggccagagagcaaccatttcttaccgggcttcaaagtcagtcagcacaagcggttactcctacatgcattggaaccagcagaagcccggacaaccccctcgcctgctgatttatctggtgagcaatctcgagtccggggtgcctgccaggttttcaggatcagggtctggtacagactttacactcaatattcatcctgttgaggaagaagacgctgcaacatactattgccagcatatcagagaactcaccagaagcggaggtggaccatcatggaaatga

SEQ ID NO: 36 - DNA, 342 bp, Artificial Sequence
  A synthetic oligonucleotide
ggcgggggcggctctgacattgtaatgacacagagtcccgcttcacttgcagtcagcctggggcaaagggcgactattagttaccgcgcatctaaaagcgtgagcacctctggctattcttatatgcattggaaccagcagaaacccggccaacccccccgactgctcatctaccttgttagcaacctggaaagcggcgtgcccgcacggttcagcggcagcgggtcaggtaccgactttactctgaatatccaccctgttgaggaggaggatgcggccacatattactgccagcacatacgggagctgactcgatcaggagggggcccctcctggaagtga

SEQ ID NO: 37 - DNA, 342 bp, Artificial Sequence
  A synthetic oligonucleotide
ggaggtggaggatcagatattgttatgactcaaagcccagcatcattggctgtatctcttggacagagagcaactatttcttaccgtgctagtaagtcagttagtacctctggttattcatatatgcattggaatcaacagaagcctggtcaacctccaagactgctaatttatctcgtttctaatcttgaatctggagtacctgctagattttcaggtagtggaagcgggaccgatttcacattgaacattcacccggtggaggaagaagatgctgctacgtattattgtcaacatattagagagcttacaagatctggggggggaccatcatggaaataa

SEQ ID NO: 38 - DNA, 342 bp, Artificial Sequence
  A synthetic oligonucleotide
ggaggaggaggaagtgacattgtgatgactcaatcacctgctagccttgcagtgtctttggggcaacgtgctactataagttatagagcatctaaatctgtgtctacaagtgggtactcatatatgcattggaatcaacaaaagccaggacaaccacctcgtttgttgatttatctagttagcaacctagagagcggagttcctgcaaggtttagcggatctgggagtggcacagatttcactcttaacatccatccagttgaggaagaggatgctgctacttattactgtcaacatattcgagaactaacccgttctggggggggtccatcctggaaataa

SEQ ID NO: 39 - DNA, 342 bp, Artificial Sequence
  A synthetic oligonucleotide
ggtgggggtggatctgatattgttatgacgcaatctcctgcttctttagcagtgtcattgggtcagagagctacgatcagttatagagctagtaagagtgtttctacgtctggttattcttatatgcattggaatcaacagaagcctggccaacctccgagactactcatctacctcgtctctaacttggaaagtggagtcccagcaagatttagtggctccggttcaggaaccgattttactttaaatatccatcccgtcgaagaggaggacgcagcaacctactattgtcagcatattagggagttaactcgaagtggtggaggtccatcttggaaataa

SEQ ID NO: 40 - DNA, 342 bp, Artificial Sequence
  A synthetic oligonucleotide
ggcggaggaggttctgatattgttatgactcagtctccagcttcactagctgtgtcattgggccagcgagcaactatttcatatagagcctctaagagtgtgtcaacatccggatatagttatatgcattggaatcagcaaaaacctgggcagccgccaaggcttcttatttacctagtttcaaatctagaatcaggtgtgcctgctagattttcaggatccggtagcggtactgattttactttaaatattcaccccgttgaagaggaagatgcagcaacctattattgtcaacatattagagaactcacaagatccggaggtggaccgtcttggaaatga

SEQ ID NO: 41 - DNA, 342 bp, Artificial Sequence
  A synthetic oligonucleotide
gggggaggtggttctgatattgtaatgacacagtccccagcatccttggcagtcagtttagggcaaagagctacaatcagttaccgagcttccaaaagcgtatctacttctggctacagctatatgcattggaatcagcagaagcctggtcagcctcctaggttgcttatatatttggtctctaacttagaatcaggggttccggcaagattctcaggatcagggtcaggaaccgattttactctgaatatccatcctgttgaggaagaggacgctgctacctattattgccaacatattagggaacttacgagatccggtggaggtcctagctggaaatga

SEQ ID NO: 42 - DNA, 342 bp, Artificial Sequence
  A synthetic oligonucleotide
ggtggagggggttcagatattgttatgactcagagtcctgcttcattggctgttagcctaggccagcgtgcaactatcagttatcgtgcttccaaaagcgtgtccacttcaggttacagttatatgcattggaaccaacaaaaaccaggacagccaccacgtctacttatatacttggtcagcaatctggaaagtggcgttccagctcgtttcagcggttcaggctctgggacagatttcaccctcaatattcacccagtagaagaggaagacgccgctacgtattattgccagcatattcgtgaattaactaggtctggtggcggaccatcttggaagtag

SEQ ID NO: 43 - DNA, 342 bp, Artificial Sequence
  A synthetic oligonucleotide
ggaggaggaggtagcgatattgtgatgactcaatctccagcatccttggccgtgtctttgggccagagggccacaatttcctacagggctagcaagagtgttagtacgtcaggatatagttatatgcattggaatcagcagaagccagggcagcctccaaggcttcttatctatcttgtctctaatttggaatcaggtgtcccagcccgtttttctggaagtggtagtggtacggattttacattaaatatccacccagtggaagaagaagatgccgcaacgtactattgccagcatatcagggagttgactagatcaggcgggggcccatcatggaagtga

SEQ ID NO: 44 - DNA, 342 bp, Artificial Sequence
  A synthetic oligonucleotide
ggaggtggaggatctgacattgttatgacccagtccccggcttcccttgcagtatcacttggacagcgtgcaacgatttcttatagagctagtaagagcgtgtctacatcaggatattcctacatgcattggaatcagcaaaagcctggtcagcctccaagactgctaatttatttggtcagtaatctcgaatctggtgttcccgctcggtttagcggatccggaagtggaaccgatttcacattgaatatccatccggtggaagaagaggacgctgctacatattactgccagcacatacgagagttaaccagaagtggaggcggtccctcttggaaatga

SEQ ID NO: 45 - DNA, 342 bp, Artificial Sequence
  A synthetic oligonucleotide
ggtggagggggttcagatatcgtgatgacgcagtcccccgcatcacttgcagttagtttggggcagcgtgcaacaatcagctatcgtgcatctaaaagtgtctcaacgtcaggttattcatatatgcattggaaccaacaaaagccaggacagccacctcggttgctgatatacctagtatcaaatctagagagcggagtgccggctagatttagtggtagtggttctgggacagatttcactcttaatatccacccggtggaagaggaagatgcagcaacttattactgccaacatatacgtgagcttacgagaagcggtggaggtccttcctggaaataa

SEQ ID NO: 46 - DNA, 342 bp, Artificial Sequence
  A synthetic oligonucleotide
ggcggcggtggatcagatattgttatgacacagagtcctgcatctcttgccgtttcattgggccaacgggccacaatttcatatagagctagcaagtccgtctccacgtccggatacagctatatgcattggaatcagcagaaaccaggacagcctcctagacttttaatttatttggtatcaaatcttgaaagcggagttcccgccaggttcagtggatctggttctgggaccgatttcacccttaatatacaccctgttgaagaggaagatgccgccacttactattgtcagcatattagggagctaactcgttctggaggaggaccttcatggaaataa

SEQ ID NO: 47 - skipped

SEQ ID NO: 48 - DNA, 408 bp, Mus musculus; identical to SEQ ID NO: 7

SEQ ID NO: 49 - DNA, 408 bp, Artificial Sequence
  A synthetic oligonucleotide
ggatcacaggtaaagctgcaggagtccggggctgagctggctaaacttggcgctagtgttaagatgagctgcaaggcatccgggtacacctttacgaaatactggatgcactggataaagcagcgccctggccaggggctggagtggatcggctacatcaacccaagtacagggtactcagagaataatcaaaagttcaagggcaaagccatcctgacagcagacaagagctcttcaaccgcatacatgcagctcagtagccttacatcagatgattcagcagtgtactattgtgtccgaggttacgactcccattactacgtcatggactattggggccagggtacatctgtgaccgtgtcatccgctaagacgacaccccccagcgtctatggcggcggcggtagc

SEQ ID NO: 50 - DNA, 408 bp, Artificial Sequence
  A synthetic oligonucleotide
ggaagccaggtcaagcttcaggagtcaggcgctgaactggctaagctgggcgccagtgtgaagatgtcatgtaaagcatccggatatacattcaccaagtactggatgcactggatcaagcagcgacccggccaagggcttgagtggataggttacattaacccaagcacgggatactctgaaaataaccaaaaatttaaggggaaggccatcctgacggcagacaagtcctccagtaccgcctatatgcagctgtcatctctgacatctgacgacagtgccgtgtactattgtgttaggggatacgattcccattattacgtgatggattactggggacagggcacgtctgtgacagtgtccagcgccaagacaacgccgccttccgtgtatggcggtgggggcagc

SEQ ID NO: 51 - DNA, 408 bp, Artificial Sequence
  A synthetic oligonucleotide
gggtcacaggtgaagctgcaggaaagtggagcagagctggctaagttgggcgcctctgtcaaaatgagctgtaaagctagcggctatacctttaccaagtactggatgcactggatcaagcagcggcccggtcaggggttggagtggatcggttatataaacccaagcacggggtacagcgagaacaaccaaaagtttaagggaaaagcaattctcacagctgataaatctagctctaccgcctatatgcaattgagttccctgacgtctgatgacagcgcggtttattactgtgtgagggggtacgacagtcattattacgtcatggactactggggtcagggaacaagcgttacagtcagcagcgctaaaacaactccccctagtgtgtatggtggcggcggaagt

SEQ ID NO: 52 - DNA, 408 bp, Artificial Sequence
  A synthetic oligonucleotide
gggtctcaggtgaagcttcaggagtccggagctgagttggcgaagttgggggcatcagttaaaatgtcttgcaaggcctccggttatacctttacaaagtattggatgcactggatcaagcagagacctggccagggcttggaatggattgggtacatcaacccaagtacagggtattccgagaacaaccagaagtttaaagggaaggccattctgaccgcggacaaatcttcttcaacagcctacatgcagctgagcagtctcacgagcgatgacagtgcagtttactattgcgtgcggggatatgattcccactactacgtcatggattattggggccagggtacatcagtgaccgtatcaagtgcaaaaacgactcctccgagcgtgtatggaggtggcggctca

SEQ ID NO: 53 - DNA, 408 bp, Artificial Sequence
  A synthetic oligonucleotide
gggtcacaggttaagctgcaagaatccggggccgagctggccaagcttggggcatcagttaagatgagttgcaaagcctcaggctatactttcactaagtattggatgcactggatcaagcagagaccaggccagggattggaatggatcggatacattaacccatctaccggatattccgaaaacaaccagaagtttaaagggaaagcaattctgacagccgataagtcttcctccaccgcgtacatgcagctgtctagtctcactagtgacgactccgctgtttactactgtgtccggggctacgactctcactattacgtgatggactactggggccaaggtacctctgtcacggtttctagcgccaagaccacaccgccgtcagtgtacggaggtggaggctct

SEQ ID NO: 54 - DNA, 408 bp, Artificial Sequence
  A synthetic oligonucleotide
ggctcccaggtcaagcttcaagaatccggcgcagagctcgccaagctgggtgccagcgtaaagatgagctgtaaagcctctggatacacattcacaaagtattggatgcactggataaagcagcgcccaggccagggcctcgaatggattggttatattaacccgagtaccggctacagcgaaaataaccagaaattcaagggaaaagcgatcctgacggctgataaaagttcctctacagcttacatgcagctgtcttcccttaccagcgatgactctgcagtttactattgcgtgcgggggtacgatagtcattactatgtcatggattattggggacaaggaacctcagtaacagtgtcctccgcaaagaccacgccgcctagtgtgtatggcggcggtggcagc

SEQ ID NO: 55 - DNA, 408 bp, Artificial Sequence
  A synthetic oligonucleotide
ggtagccaggtgaaactgcaggaatccggcgccgaactggccaagctcggcgcctctgttaaaatgtcatgtaaagcaagtggatacacgttcactaagtactggatgcactggataaagcagcgcccgggccagggcttggagtggatcggatacatcaatccaagtactgggtattcagagaataatcagaaatttaagggcaaagcaattctgactgccgataaatcttccagtaccgcctatatgcagctgtcttcacttaccagcgatgattcagccgtttattactgcgtgcgggggtacgactcacattattacgtcatggattactggggtcagggcacctcagttacagtgagttccgctaagaccacacctccaagcgtgtacggtgggggggggtcc

SEQ ID NO: 56 - DNA, 408 bp, Artificial Sequence
  A synthetic oligonucleotide
ggatctcaggtaaagctgcaggagagtggtgctgaactcgcaaagctcggtgcaagcgtgaaaatgagttgtaaagcctcagggtacaccttcaccaagtattggatgcactggataaagcagcggccggggcagggccttgagtggatcggttacatcaatccaagcacaggatattccgagaacaaccaaaagttcaagggcaaggcgattctgacagccgataaaagctcctcaacggcctatatgcagcttagtagcctcacaagtgacgattctgctgtgtattattgtgtccgggggtatgactctcactattacgtgatggactactggggacagggcacgtcagttacagtctcatcagcaaaaacaacaccaccaagcgtgtatggtggtggcggctct

SEQ ID NO: 57 - DNA, 408 bp, Artificial Sequence
  A synthetic oligonucleotide
ggttcacaggtgaagctccaagagagtggtgccgagctggctaagttgggagcatctgtgaagatgtcttgcaaagcaagcgggtacacttttacaaaatattggatgcactggattaagcagcgccccggacagggattggaatggattgggtatatcaacccctccaccggctattccgagaacaaccagaaatttaagggcaaggcgatactcacagcagataagtcctcaagcacagcctatatgcagcttagttcactgacgtctgacgattctgccgtgtattactgtgtgcgaggctatgacagccactactacgtgatggattattggggccagggaacatcagtgacagtaagttctgccaaaacaaccccaccctctgtgtatggtggaggtggctca

SEQ ID NO: 58 - DNA, 408 bp, Artificial Sequence
  A synthetic oligonucleotide
ggtagccaggtaaagctgcaggagagcggtgccgaactggccaagctgggggcctccgtgaagatgtcatgcaaagctagcgggtacacctttacaaaatactggatgcattggattaaacagaggcctggccagggcttggagtggatcggctatatcaatccatctacgggttactccgaaaacaatcagaagttcaagggcaaggccattctgaccgccgacaagagctcttcaacagcctatatgcagttgagctcattgacgagtgacgacagtgctgtctactattgtgtgcgcggatacgacagtcattactatgtaatggattactggggccagggaaccagcgtgactgtgtccagtgccaagactacccctcctagtgtgtacggcggcggcggcagt

SEQ ID NO: 59 - DNA, 408 bp, Artificial Sequence
  A synthetic oligonucleotide
ggaagccaggtgaagctccaagagagtggtgcagagcttgctaaactgggtgcctcagtcaagatgagctgtaaggccagtggttacacctttactaagtactggatgcactggatcaaacaaagaccaggtcaaggtttggagtggattggctatatcaacccgtctacaggatatagcgaaaacaatcagaaatttaaaggaaaggctatcttgacggctgacaaaagtagttctactgcctatatgcaattatcatcattgacaagcgacgattctgcagtttactactgcgtgcgaggatacgattcccactattacgttatggattattggggtcagggtacaagtgttacagtttcctctgcaaaaacaactccaccatctgtttatggaggtggcggatct

SEQ ID NO: 60 - DNA, 408 bp, Artificial Sequence
  A synthetic oligonucleotide
gggtctcaagtgaagttgcaggaatctggagctgagttggctaagttgggtgcttccgtcaagatgagttgtaaagcttccggatacactttcaccaaatactggatgcattggatcaaacagcggccgggtcagggtctagagtggattgggtatattaatccgtccaccggatacagcgagaacaatcagaaatttaagggaaaggcaatacttactgctgataagagctcaagtactgcatatatgcagttgtctagtcttacctcagatgacagtgctgtgtattattgcgttcgagggtacgattcacattactatgtaatggactactggggacagggcacgagtgttactgtttcaagcgctaagacaacccctccttccgtgtacggcggaggaggcagc

SEQ ID NO: 61 - DNA, 408 bp, Artificial Sequence
  A synthetic oligonucleotide
gggagtcaagtgaagcttcaagagagtggagcagagctagcaaagctcggcgcatcagttaagatgtcatgtaaagccagcggttatacttttactaagtattggatgcattggataaaacaaaggccaggtcaaggcctggagtggatcggctacatcaatccatcaacaggttattctgaaaataatcagaaattcaaaggaaaagctattttgacggcagacaaaagctccagtactgcttatatgcaattgtcttcccttacgtcagatgattcagctgtttactactgcgtgagaggttacgatagtcactattacgtaatggattactggggtcaaggaacctctgtaactgtttcttctgcaaagactactcctccaagcgtctacggaggtggtggtagt

SEQ ID NO: 62 - DNA, 408 bp, Artificial Sequence
  A synthetic oligonucleotide
ggtagccaagtgaagcttcaggagagtggtgctgaactcgctaaactgggtgcttctgtcaagatgagttgtaaagctagcggttacacattcactaaatattggatgcactggatcaaacagagacctggtcaaggcctcgaatggataggatacattaaccctagtacaggttactcagagaataatcaaaagttcaaaggcaaagcaattttgactgctgacaaatcatcttctacggcctacatgcaactctcttcattgactagtgacgattccgctgtgtactattgtgtgagaggatatgactctcactattatgttatggattactggggacaagggacatctgttacagtatcttcagcaaagactactcctccctcagtttatggtggaggaggttct

SEQ ID NO: 63 - DNA, 408 bp, Artificial Sequence
  A synthetic oligonucleotide
ggctctcaagttaaactacaggaatccggagccgagttggctaaacttggggctagtgttaaaatgtcctgtaaggcctctggttacacatttaccaagtattggatgcattggattaagcagaggcctggtcaaggactcgaatggatcggttatattaatccaagcacaggatattctgagaataaccaaaaattcaaaggtaaagcaatcctgacagcagataaaagcagcagcaccgcatatatgcagttgagtagcttgacatcagatgatagtgctgtttactattgcgtacgtggctacgattcccactactacgtcatggattattggggtcaaggcacatcagttacggtatcatctgctaagacaacacctcctagtgtatatggaggaggaggcagt

SEQ ID NO: 64 - DNA, 408 bp, Artificial Sequence
  A synthetic oligonucleotide
ggcagtcaggttaaactgcaggagagcggtgctgagttagcaaagttgggtgcatcagtaaagatgtcttgtaaagcaagtggctatacatttacgaaatattggatgcactggattaagcaacgaccaggacaaggccttgaatggataggatatataaatccctcaaccggctattccgagaataaccaaaagttcaagggtaaagctattttgactgctgataaatcttcttcaaccgcctatatgcaactatcatctctgacttctgacgattccgctgtgtattattgtgttcgaggttacgattctcattactatgtgatggactactggggacaaggtacttccgttacggtctcatctgctaagactacccccccatctgtgtatggaggtggtggatcc

SEQ ID NO: 65 - DNA, 408 bp, Artificial Sequence
  A synthetic oligonucleotide
ggcagccaggtcaagttgcaagaatctggtgctgaactggccaaattgggggcatctgtgaaaatgagttgcaaagcctccgggtacacatttacaaagtattggatgcattggataaagcagagacctgggcaaggattggagtggattggttacataaacccttctactggatattctgagaataatcagaagttcaaaggtaaggcaattcttacagccgataaaagctcaagtacggcctatatgcaactctcaagcctgacatctgatgatagcgcagtgtattactgcgttagaggatacgatagccactactacgtaatggattactggggccaaggtacatctgttacagtgtctagtgcaaaaactacacctccctcagtttacggagggggaggtagc

SEQ ID NO: 66 - DNA, 408 bp, Artificial Sequence
  A synthetic oligonucleotide
ggttctcaagtaaaattacaagagagcggagctgagcttgctaagctcggcgcttcagttaaaatgtcttgtaaggctagtgggtacacttttactaaatactggatgcattggattaaacagagaccagggcagggattagaatggatcggatatataaatcctagcacggggtactctgagaataatcagaaattcaaaggcaaggctatattgacggcagataagagtagctctactgcctacatgcaactgtccagcctaactagtgatgatagtgctgtttactactgtgttcgtggttatgacagccactactatgtaatggattactggggtcaaggtacaagtgttactgtttctagtgctaagaccacgccaccgtctgtttatggtggcggtgggtca

SEQ ID NO: 67 - DNA, 408 bp, Artificial Sequence
  A synthetic oligonucleotide
ggatcccaagtgaagttgcaagaaagcggtgcagagttagctaaacttggagcctctgttaaaatgagttgcaaagcctccggatatacttttaccaaatactggatgcattggattaaacagaggcccggtcaaggcctggagtggattggatatatcaacccaagcactggctattctgagaataaccagaaatttaagggaaaggccatcttgaccgctgataagtcttcatcaactgcatatatgcagctcagcagccttacgtccgatgacagcgctgtgtattactgtgtgcgaggttatgattcccattattacgtaatggattattggggtcaaggaacaagtgttacagtttcaagtgcaaaaacgacacctccttctgtatatggaggtggaggctca

SEQ ID NO: 68 - DNA, 408 bp, Artificial Sequence
  A synthetic oligonucleotide
ggttctcaggtaaaattacaagaaagtggagcagaattagctaaattgggagcaagcgtgaagatgtcatgcaaggcaagcggttatactttcactaagtattggatgcattggatcaagcagcgtcctggtcagggattggagtggataggatatattaatccttctacaggctattcagaaaacaaccaaaagtttaaaggtaaggctatactcactgcagataaaagcagttccactgcttacatgcagctcagtagtcttacaagcgatgactctgctgtgtactattgtgtaaggggctatgatagccattactacgtaatggactactgggggcaaggtacttctgtgactgttagcagtgctaaaactactccaccgtcagtgtacggtggtggaggttca

SEQ ID NO: 69 - DNA, 342 bp, Mus musculus; identical to SEQ ID NO: 8

SEQ ID NO: 70 - DNA, 342 bp, Artificial Sequence
  A synthetic oligonucleotide
ggcggtggcggcagcgatatcgtgcttacgcagagtccagcatcactggcagtctccctgggtcagagggccacaatctcctatagagcctccaaaagtgtttcaactagcggatactcttatatgcattggaatcagcaaaaacccggtcagccgcctagactgcttatctatctggtgtccaacctcgaatccggggtccctgcccgattctctggctcaggttcaggcaccgatttcacactgaacattcatccggtcgaggaggaggatgccgccacttattactgccagcatattcgggagctcacacgcagcgagggggggccttcttggaagtga

SEQ ID NO: 71 - DNA, 342 bp, Artificial Sequence
  A synthetic oligonucleotide
ggggggggaggctcagatatagttttgacacagagtcctgccagcctggcagtttccctgggtcagcgggccaccatctcatacagggcttcaaagagtgtgtcaacctctggctatagttatatgcattggaatcagcagaaaccaggacagcccccgaggctgcttatttatctggtgagcaaccttgaaagtggcgttcctgcccgcttctcagggtccggtagcggcacagattttaccctgaacatacatcccgtcgaggaggaggatgcagctacctactattgtcagcacattagagagctgactcgctccgagggagggccaagctggaagtag

SEQ ID NO: 72 - DNA, 342 bp, Artificial Sequence
  A synthetic oligonucleotide
ggcggaggaggcagcgatattgtactgactcagagcccggcaagcctggctgttagcttggggcaacgcgccacaataagttaccgcgcctctaagagtgtgtcaacctcaggctattcttacatgcactggaatcaacagaagccgggccagcccccgaggctgctgatctacctggtaagcaacctcgagagtggagtcccggctagattttcaggctctgggtctggcacagactttacgttgaacattcaccctgttgaagaggaggatgctgctacatattattgccagcacatcagggagctgactagatcagaggggggcccctcttggaagtag

SEQ ID NO: 73 - DNA, 342 bp, Artificial Sequence
  A synthetic oligonucleotide
ggaggcggcgggagcgatatcgtgcttactcaatctcccgcatctctggctgtgtctctcggacagagggctacaatttcctatagggcatccaaaagcgtttccacaagtggctactcttacatgcattggaaccagcagaagccgggccaaccgcctaggctgctgatttacctggtatccaatcttgagagcggagtgcctgcccggtttagcggatcaggctccggtaccgatttcaccctcaatattcatccggtggaagaggaggacgctgcaacctactactgtcagcatatccgcgaacttaccagatccgagggagggccttcctggaaatga

SEQ ID NO: 74 - DNA, 342 bp, Artificial Sequence
  A synthetic oligonucleotide
ggcggtgggggctccgacatagtgttgacccaatcccctgcctccctcgccgtgtctctcggccagagggccacaatttcctacagagcaagcaagtccgtgtccacctctggatactcatatatgcactggaatcagcaaaagcccggccaacctcccagacttcttatctatcttgtgtctaacctggaatctggcgtccccgcgcggttttcaggctccggatcaggaaccgattttacactgaacatccaccctgtggaagaggaagatgctgcaacttactactgtcagcatattcgagagttgaccagatccgagggtggccccagttggaaatag

SEQ ID NO: 75 - DNA, 342 bp, Artificial Sequence
  A synthetic oligonucleotide
gggggaggagggtcagacattgttctgacacagtcaccggcttcccttgctgtgagcctgggccagcgggctactatctcctacagggctagcaaatcagtctcaacatccggatattcctacatgcattggaaccaacaaaaaccagggcagccgccaagactcctgatctatttggtgagcaatctggaatctggagtgccagcccgcttttccggaagcggttctggaacagacttcactctgaatattcaccccgtcgaagaggaggacgccgctacgtactactgtcagcatattcgcgagttgaccagatctgagggaggtccttcctggaagtaa

SEQ ID NO: 76 - DNA, 342 bp, Artificial Sequence
  A synthetic oligonucleotide
gggggcggcgggtccgatattgttttgacccagtctcccgcatcacttgcagtctccctggggcagcgagccaccatttcctatcgagctagtaaatctgtcagtacatctggatatagttatatgcattggaaccagcaaaagccaggacagccccctcggctgctgatatacctggtgtcaaacctggagtctggggttcctgcccggttttccggatctggctccgggacggactttacactgaatatccaccccgttgaggaagaggatgccgccacctactattgccagcatatccgcgaacttacccgaagtgaggggggcccctcctggaagtaa

SEQ ID NO: 77 - DNA, 342 bp, Artificial Sequence
  A synthetic oligonucleotide
ggtggtggcggttccgacatagtcctgacccagagcccagcatccctggcagttagtcttgggcagcgggccaccatcagctaccgcgcaagcaagtccgttagtacttccggatactcatacatgcactggaatcagcaaaagccaggtcagccccccaggctgctgatctatctggtgtctaacctggagagtggcgtaccagcacgatttagcggctctgggagcggcactgatttcactctgaatattcacccggtggaggaggaggatgctgctacatactactgtcagcacattcgggaactgaccaggtctgaaggaggtccaagttggaagtga

SEQ ID NO: 78 - DNA, 342 bp, Artificial Sequence
  A synthetic oligonucleotide
ggaggcggtggaagcgacatcgttctgactcagagcccggcatccttggcagtcagcttgggccagcgggccacaatctcataccgcgcttccaaatcagtcagcacctccggttacagctatatgcactggaaccaacagaaaccaggacaaccccctaggctgctcatctatcttgtttctaacctggaatccggagtgcctgcccggttctcaggttccggaagtggaactgatttcactctcaatatccatccagtagaggaggaggatgctgctacatactattgccagcacatccgcgagctgaccagatccgaaggaggccccagttggaagtga

SEQ ID NO: 79 - DNA, 342 bp, Artificial Sequence
  A synthetic oligonucleotide
gggggggggggcagcgacatcgtgctgacccagtctccagcttcactggccgtgagtctgggccaacgggctaccatttcttatcgggcctctaagtccgtttcaacctcagggtatagctatatgcactggaaccagcagaaaccaggacagcccccacgactcctgatctacttggtcagtaatctcgagagtggcgtcccggcacgattcagcggctctggctcaggcactgacttcaccctgaatatccatccagttgaagaagaggacgctgcgacctactactgccaacatatcagggaattgactcggagcgagggaggccccagttggaagtaa

SEQ ID NO: 80 - DNA, 342 bp, Artificial Sequence
  A synthetic oligonucleotide
ggaggcggaggttctgacatcgtcttaacccagtctcctgcatctctcgcagttagcttgggtcaaagggcaactatttcttatcgtgccagtaaatcagtatctacatctggatattcctatatgcactggaatcaacagaaacctggacagccaccaaggcttcttatatatctagtatccaacttggaaagcggtgttcctgccagattcagtgggtccggtagcggtactgatttcaccttgaatatccatcccgtagaagaggaagatgctgccacctattactgtcagcacattcgtgagctcactagaagcgaggggggacctagttggaagtag

SEQ ID NO: 81 - DNA, 342 bp, Artificial Sequence
  A synthetic oligonucleotide
ggtggtgggggctcagatatagtgcttactcaaagcccagcatcattggccgttagtttaggacagagggctactatttcataccgtgcatcaaaatctgtatccacctctggttacagttacatgcattggaaccagcagaaaccaggccagcccccgaggcttctgatctaccttgttagcaatctggaaagcggagtcccagctaggttttcaggtagtggcagtggtacagattttactttgaatattcatcctgtcgaagaggaagatgcagctacctattattgtcagcatatccgtgagctaacacgatctgaaggcggcccttcctggaagtga

SEQ ID NO: 82 - DNA, 342 bp, Artificial Sequence
  A synthetic oligonucleotide
ggtggcggaggatcagacattgtgcttacacaatcaccagcatcattagctgtttccttagggcagcgtgctaccatatcctatagggcctcaaagtctgtttcaacttcaggatactcatacatgcattggaaccaacagaagccaggacaaccgccaagactgcttatttatttagtttcaaaccttgaatccggtgtgcctgcacgtttttcaggtagtgggtcaggaacagattttacacttaatatacaccctgttgaagaggaggacgccgcaacttactactgccaacatattcgtgaacttacacgttcagagggaggtccaagctggaagtaa

SEQ ID NO: 83 - DNA, 342 bp, Artificial Sequence
  A synthetic oligonucleotide
ggtggtggtggatccgatattgttcttacacaaagtccggcatcactggctgtgtctttaggtcaaagggctactatttcatatagggcaagtaagagtgtgtcaacatccggctactcatacatgcactggaaccaacaaaaaccagggcagccccctcggttgttaatttatttggtgtcaaatctcgagagtggtgttccggcaagattttctggatcagggtcagggactgattttacattaaacatccatcccgtcgaggaagaggatgccgcaacgtattactgccaacatattcgagagttgaccagatccgaaggtgggccctcatggaaatga

SEQ ID NO: 84 - DNA, 342 bp, Artificial Sequence
  A synthetic oligonucleotide
gggggcgggggatccgatatcgttctaactcaatctccagctagtttagccgtgtccttaggacagagagcaactattagttatagagcaagcaaatctgtgtctacatcaggatattcatatatgcattggaatcaacaaaagccgggtcaacctccaagattactcatctatcttgtttctaatttagagtccggtgtgcctgctcgtttcagtggaagtgggtcaggaaccgacttcactcttaatattcatccagtggaagaggaagatgcagcaacttattattgccagcacatacgggaacttactcgttccgagggtggaccttcatggaagtga

SEQ ID NO: 85 - DNA, 342 bp, Artificial Sequence
  A synthetic oligonucleotide
ggcggtggggggtctgatatagtcttaactcagtccccggcctctcttgccgttagtctgggccagagagctacaatctcatatagggcttcaaaaagtgtgtccacttcaggttactcttacatgcactggaatcaacaaaagccgggacagccacctcgtctactgatataccttgtctcaaacttggagagtggagtgccagctaggtttagtggatccggatccggtactgattttactcttaatattcatcctgttgaggaagaggacgccgcaacttattattgccaacatattagggaattaactaggtccgaaggagggccgagctggaagtag

SEQ ID NO: 86 - DNA, 342 bp, Artificial Sequence
  A synthetic oligonucleotide
ggtggagggggtagtgacatagttctgacacagagcccagcttcactcgctgtgtctcttggacagagggcaaccattagttaccgtgcttctaagtctgtgagtacatctggttattcatatatgcattggaatcaacaaaaacctggtcaaccaccacgacttttaatctacttagtgtctaatttggaaagcggtgttcctgccaggttttcaggttcaggaagcggtacagattttactctgaacatacacccagtggaggaagaagacgcagctacttactattgtcaacacataagggagctgacgagatctgagggcgggccttcctggaagtga

SEQ ID NO: 87 - DNA, 342 bp, Artificial Sequence
  A synthetic oligonucleotide
ggaggtgggggatctgacattgttttaacacagtctcctgccagtctcgctgtttcactgggccaacgggctactataagttacagagcatcaaaaagtgtttctacgagtggttactcttatatgcactggaaccagcagaaaccaggtcagcctcctagattacttatttaccttgtgagcaatctagagagtggtgttccagctagattctcaggttctgggtctggtaccgattttaccctaaacattcatcctgttgaagaagaagatgctgccacatattattgtcagcatatacgagagttgactaggagtgaaggcggacccagctggaagtaa

SEQ ID NO: 88 - DNA, 342 bp, Artificial Sequence
  A synthetic oligonucleotide
ggtggcggaggatccgatattgtgttgactcaatcacccgcatcactggcagtttcactggggcaacgggctactatcagttatagagcttcaaagtccgtgagtacttccggttactcttacatgcactggaaccaacaaaaacccggacaacctcctcgtcttcttatttatttggttagtaacctagaatccggtgttcctgccagattctctggtagtggttctggcaccgacttcactttgaatatacacccagtcgaagaggaagatgccgccacttattactgccaacatattcgagaattgacacgttcagagggtggaccctcatggaagtag

SEQ ID NO: 89 - DNA, 342 bp, Artificial Sequence
  A synthetic oligonucleotide
ggcggtgggggttccgatatcgtattgacccaaagtcccgctagcttggcagtctctttgggacaacgtgctactattagttaccgagcttcaaagtctgtgtccactagcggatattcttacatgcattggaaccagcagaaacccggacagcctccacgactcctaatttatttggtatcaaaccttgaatctggtgtcccagccaggttttccggaagcgggtcaggcacagattttaccttgaatatccatccagtggaagaagaagacgcagctacttactactgtcagcatattagggagctcaccaggtccgaaggaggaccaagttggaaataa

SEQ ID NO: 90 - DNA, 18959 bp, Ebola virus
cggacacacaaaaagaaagaagaatttttaggatcttttgtgtgcgaataactatgaggaagattaataattttcctctcattgaaatttatatcggaatttaaattgaaattgttactgtaatcacacctggtttgtttcagagccacatcacaaagatagagaacaacctaggtctccgaagggagcaagggcatcagtgtgctcagttgaaaatcccttgtcaacacctaggtcttatcacatcacaagttccacctcagactctgcagggtgatccaacaaccttaatagaaacattattgttaaaggacagcattagttcacagtcaaacaagcaagattgagaattaaccttggttttgaacttgaacacttaggggattgaagattcaacaaccctaaagcttggggtaaaacattggaaatagttaaaagacaaattgctcggaatcacaaaattccgagtatggattctcgtcctcagaaaatctggatggcgccgagtctcactgaatctgacatggattaccacaagatcttgacagcaggtctgtccgttcaacaggggattgttcggcaaagagtcatcccagtgtatcaagtaaacaatcttgaagaaatttgccaacttatcatacaggcctttgaagcaggtgttgattttcaagagagtgcggacagtttccttctcatgctttgtcttcatcatgcgtaccagggagattacaaacttttcttggaaagtggcgcagtcaagtatttggaagggcacgggttccgttttgaagtcaagaagcgtgatggagtgaagcgccttgaggaattgctgccagcagtatctagtggaaaaaacattaagagaacacttgctgccatgccggaagaggagacaactgaagctaatgccggtcagtttctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttctgatcaaatttctcctaatacaccaagggatgcacatggttgccgggcatgatgccaacgatgctgtgatttcaaattcagtggctcaagctcgtttttcaggcttattgattgtcaaaacagtacttgatcatatcctacaaaagacagaacgaggagttcgtctccatcctcttgcaaggaccgccaaggtaaaaaatgaggtgaactcctttaaggctgcactcagctccctggccaagcatggagagtatgctcctttcgcccgacttttgaacctttctggagtaaataatcttgagcatggtcttttccctcaactatcggcaattgcactcggagtcgccacagcacacgggagtaccctcgcaggagtaaatgttggagaacagtatcaacaactcagagaggctgccactgaggctgagaagcaactccaacaatatgcagagtctcgcgaacttgaccatcttggacttgatgatcaggaaaagaaaattcttatgaacttccatcagaaaaagaacgaaatcagcttccagcaaacaaacgctatggtaactctaagaaaagagcgcctggccaagctgacagaagctatcactgctgcgtcactgcccaaaacaagtggacattacgatgatgatgacgacattccctttccaggacccatcaatgatgacgacaatcctggccatcaagatgatgatccgactgactcacaggatacgaccattcccgatgtggtggttgatcccgatgatggaagctacggcgaataccagagttactcggaaaacggcatgaatgcaccagatgacttggtcctattcgatctagacgaggacgacgaggacactaagccagtgcctaatagatcgaccaagggtggacaacagaagaacagtcaaaagggccagcatatagagggcagacagacacaatccaggccaattcaaaatgtcccaggccctcacagaacaatccaccacgccagtgcgccactcacggacaatgacagaagaaatgaaccctccggctcaaccagccctcgcatgctgacaccaattaacgaagaggcagacccactggacgatgccgacgacgagacgtctagccttccgcccttggagtcagatgatgaagagcaggacagggacggaacttccaaccgcacacccactgtcgccccaccggctcccgtatacagagatcactctgaaaagaaagaactcccgcaagacgagcaacaagatcaggaccacactcaagaggccaggaaccaggacagtgacaacacccagtcagaacactcttttgaggagatgtatcgccacattctaagatcacaggggccatttgatgctgttttgtattatcatatgatgaaggatgagcctgtagttttcagtaccagtgatggcaaagagtacacgtatccagactcccttgaagaggaatatccaccatggctcactgaaaaagaggctatgaatgaagagaatagatttgttacattggatggtcaacaattttattggccggtgatgaatcacaagaataaattcatggcaatcctgcaacatcatcagtgaatgagcatggaacaatgggatgattcaaccgacaaatagctaacattaagtagtcaaggaacgaaaacaggaagaatttttgatgtctaaggtgtgaattattatcacaataaaagtgattcttatttttgaatttaaagctagcttattattactagccgtttttcaaagttcaatttgagtcttaatgcaaataggcgttaagccacagttatagccataattgtaactcaatattctaactagcgatttatctaaattaaattacattatgcttttataacttacctactagcctgcccaacatttacacgatcgttttataattaagaaaaaactaatgatgaagattaaaaccttcatcatccttacgtcaattgaattctctagcactcgaagcttattgtcttcaatgtaaaagaaaagctggtctaacaagatgacaactagaacaaagggcaggggccatactgcggccacgactcaaaacgacagaatgccaggccctgagctttcgggctggatctctgagcagctaatgaccggaagaattcctgtaagcgacatcttctgtgatattgagaacaatccaggattatgctacgcatcccaaatgcaacaaacgaagccaaacccgaagacgcgcaacagtcaaacccaaacggacccaatttgcaatcatagttttgaggaggtagtacaaacattggcttcattggctactgttgtgcaacaacaaaccatcgcatcagaatcattagaacaacgcattacgagtcttgagaatggtctaaagccagtttatgatatggcaaaaacaatctcctcattgaacagggtttgtgctgagatggttgcaaaatatgatcttctggtgatgacaaccggtcgggcaacagcaaccgctgcggcaactgaggcttattgggccgaacatggtcaaccaccacctggaccatcactttatgaagaaagtgcgattcggggtaagattgaatctagagatgagaccgtccctcaaagtgttagggaggcattcaacaatctaaacagtaccacttcactaactgaggaaaattttgggaaacctgacatttcggcaaaggatttgagaaacattatgtatgatcacttgcctggttttggaactgctttccaccaattagtacaagtgatttgtaaattgggaaaagatagcaactcattggacatcattcatgctgagttccaggccagcctggctgaaggagactctcctcaatgtgccctaattcaaattacaaaaagagttccaatcttccaagatgctgctccacctgtcatccacatccgctctcgaggtgacattccccgagcttgccagaaaagcttgcgtccagtcccaccatcgcccaagattgatcgaggttgggtatgtgtttttcagcttcaagatggtaaaacacttggactcaaaatttgagccaatctcccttccctccgaaagaggcgaataatagcagaggcttcaactgctgaactatagggtacgttacattaatgatacacttgtgagtatcagccctggataatataagtcaattaaacgaccaagataaaattgttcatatctcgctagcagcttaaaatataaatgtaataggagctatatctctgacagtattataatcaattgttattaagtaacccaaaccaaaagtgatgaagattaagaaaaacctacctcggctgagagagtgttttttcattaaccttcatcttgtaaacgttgagcaaaattgttaaaaatatgaggcgggttatattgcctactgctcctcctgaatatatggaggccatataccctgtcaggtcaaattcaacaattgctagaggtggcaacagcaatacaggcttcctgacaccggagtcagtcaatggggacactccatcgaatccactcaggccaattgccgatgacaccatcgaccatgccagccacacaccaggcagtgtgtcatcagcattcatccttgaagctatggtgaatgtcatatcgggccccaaagtgctaatgaagcaaattccaatttggcttcctctaggtgtcgctgatcaaaagacctacagctttgactcaactacggccgccatcatgcttgcttcatacactatcacccatttcggcaaggcaaccaatccacttgtcagagtcaatcggctgggtcctggaatcccggatcatcccctcaggctcctgcgaattggaaaccaggctttcctccaggagttcgttcttccgccagtccaactaccccagtatttcacctttgatttgacagcactcaaactgatcacccaaccactgcctgctgcaacatggaccgatgacactccaacaggatcaaatggagcgttgcgtccaggaatttcatttcatccaaaacttcgccccattcttttacccaacaaaagtgggaagaaggggaacagtgccgatctaacatctccggagaaaatccaagcaataatgacttcactccaggactttaagatcgttccaattgatccaaccaaaaatatcatgggaatcgaagtgccagaaactctggtccacaagctgaccggtaagaaggtgacttctaaaaatggacaaccaatcatccctgttcttttgccaaagtacattgggttggacccggtggctccaggagacctcaccatggtaatcacacaggattgtgacacgtgtcattctcctgcaagtcttccagctgtgattgagaagtaattgcaataattgactcagatccagttttatagaatcttctcagggatagtgataacatctatttagtaatccgtccattagaggagacacttttaattgatcaatatactaaaggtgctttacaccattgtcttttttctctcctaaatgtagaacttaacaaaagactcataatatacttgtttttaaaggattgattgatgaaagatcataactaataacattacaaataatcctactataatcaatacggtgattcaaatgttaatctttctcattgcacatactttttgcccttatcctcaaattgcctgcatgcttacatctgaggatagccagtgtgacttggattggaaatgtggagaaaaaatcgggacccatttctaggttgttcacaatccaagtacagacattgcccttctaattaagaaaaaatcggcgatgaagattaagccgacagtgagcgtaatcttcatctctcttagattatttgttttccagagtaggggtcgtcaggtccttttcaatcgtgtaaccaaaataaactccactagaaggatattgtggggcaacaacacaatgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttagtttttcttcagattgcttcatggaaaagctcagcctcaaatcaatgaaaccaggatttaattatatggattacttgaatctaagattacttgacaaatgataatataatacactggagctttaaacatagccaatgtgattctaactcctttaaactcacagttaatcataaacaaggtttgacatcaatctagttatctctttgagaatgataaacttgatgaagattaagaaaaaggtaatctttcgattatctttaatcttcatccttgattctacaatcatgacagttgtctttagtgacaagggaaagaagcctttttattaagttgtaataatcagatctgcgaaccggtagagtttagttgcaacctaacacacataaagcattggtcaaaaagtcaatagaaatttaaacagtgagtggagacaacttttaaatggaagcttcatatgagagaggacgcccacgagctgccagacagcattcaagggatggacacgaccaccatgttcgagcacgatcatcatccagagagaattatcgaggtgagtaccgtcaatcaaggagcgcctcacaagtgcgcgttcctactgtatttcataagaagagagttgaaccattaacagttcctccagcacctaaagacatatgtccgaccttgaaaaaaggatttttgtgtgacagtagtttttgcaaaaaagatcaccagttggagagtttaactgatagggaattactcctactaatcgcccgtaagacttgtggatcagtagaacaacaattaaatataactgcacccaaggactcgcgcttagcaaatccaacggctgatgatttccagcaagaggaaggtccaaaaattaccttgttgacactgatcaagacggcagaacactgggcgagacaagacatcagaaccatagaggattcaaaattaagagcattgttgactctatgtgctgtgatgacgaggaaattctcaaaatcccagctgagtcttttatgtgagacacacctaaggcgcgaggggcttgggcaagatcaggcagaacccgttctcgaagtatatcaacgattacacagtgataaaggaggcagttttgaagctgcactatggcaacaatgggaccgacaatccctaattatgtttatcactgcattcttgaatattgctctccagttaccgtgtgaaagttctgctgtcgttgtttcagggttaagaacattggttcctcaatcagataatgaggaagcttcaaccaacccggggacatgctcatggtctgatgagggtaccccttaataaggctgactaaaacactatataaccttctacttgatcacaatactccgtatacctatcatcatatatttaatcaagacgatatcctttaaaacttattcagtactataatcactctcgtttcaaattaataagatgtgcatgattgccctaatatatgaagaggtatgatacaaccctaacagtgatcaaagaaaatcataatctcgtatcgctcgtaatataacctgccaagcatacctcttgcacaaagtgattcttgtacacaaataatgttttactctacaggaggtagcaacgatccatcccatcaaaaaataagtatttcatgacttactaatgatctcttaaaatattaagaaaaactgacggaacataaattctttatgcttcaagctgtggaggaggtgtttggtattggctattgttatattacaatcaataacaagcttgtaaaaatattgttcttgtttcaagaggtagattgtgaccggaaatgctaaactaatgatgaagattaatgcggaggtctgataagaataaaccttattattcagattaggccccaagaggcattcttcatctccttttagcaaagtactatttcagggtagtccaattagtggcacgtcttttagctgtatatcagtcgcccctgagatacgccacaaaagtgtctctaagctaaattggtctgtacacatcccatacattgtattaggggcaataatatctaattgaacttagccgtttaaaatttagtgcataaatctgggctaacaccaccaggtcaactccattggctgaaaagaagcttacctacaacgaacatcactttgagcgccctcacaattaaaaaataggaacgtcgttccaacaatcgagcgcaaggtttcaaggttgaactgagagtgtctagacaacaaaatattgatactccagacaccaagcaagacctgagaaaaaaccatggctaaagctacgggacgatacaatctaatatcgcccaaaaaggacctggagaaaggggttgtcttaagcgacctctgtaacttcttagttagccaaactattcaggggtggaaggtttattgggctggtattgagtttgatgtgactcacaaaggaatggccctattgcatagactgaaaactaatgactttgcccctgcatggtcaatgacaaggaatctctttcctcatttatttcaaaatccgaattccacaattgaatcaccgctgtgggcattgagagtcatccttgcagcagggatacaggaccagctgattgaccagtctttgattgaacccttagcaggagcccttggtctgatctctgattggctgctaacaaccaacactaaccatttcaacatgcgaacacaacgtgtcaaggaacaattgagcctaaaaatgctgtcgttgattcgatccaatattctcaagtttattaacaaattggatgctctacatgtcgtgaactacaacggattgttgagcagtattgaaattggaactcaaaatcatacaatcatcataactcgaactaacatgggttttctggtggagctccaagaacccgacaaatcggcaatgaaccgcatgaagcctgggccggcgaaattttccctccttcatgagtccacactgaaagcatttacacaaggatcctcgacacgaatgcaaagtttgattcttgaatttaatagctctcttgctatctaactaaggtagaatacttcatattgagctaactcatatatgctgactcaatagttatcttgacatctctgctttcataatcagatatataagcataataaataaatactcatatttcttgataatttgtttaaccacagataaatcctcactgtaagccagcttccaagttgacacccttacaaaaaccaggactcagaatccctcaaacaagagattccaagacaacatcatagaattgctttattatatgaataagcattttatcaccagaaatcctatatactaaatggttaattgtaactgaacccgcaggtcacatgtgttaggtttcacagattctatatattactaactctatactcgtaattaacattagataagtagattaagaaaaaagcctgaggaagattaagaaaaactgcttattgggtctttccgtgttttagatgaagcagttgaaattcttcctcttgatattaaatggctacacaacatacccaatacccagacgctaggttatcatcaccaattgtattggaccaatgtgacctagtcactagagcttgcgggttatattcatcatactcccttaatccgcaactacgcaactgtaaactcccgaaacatatctaccgtttgaaatacgatgtaactgttaccaagttcttgagtgatgtaccagtggcgacattgcccatagatttcatagtcccagttcttctcaaggcactgtcaggcaatggattctgtcctgttgagccgcggtgccaacagttcttagatgaaatcattaagtacacaatgcaagatgctctcttcttgaaatattatctcaaaaatgtgggtgctcaagaagactgtgttgatgaacactttcaagagaaaatcttatcttcaattcagggcaatgaatttttacatcaaatgtttttctggtatgatctggctattttaactcgaaggggtagattaaatcgaggaaactctagatcaacatggtttgttcatgatgatttaatagacatcttaggctatggggactatgttttttggaagatcccaatttcaatgttaccactgaacacacaaggaatcccccatgctgctatggactggtatcaggcatcagtattcaaagaagcggttcaagggcatacacacattgtttctgtttctactgccgacgtcttgataatgtgcaaagatttaattacatgtcgattcaacacaactctaatctcaaaaatagcagagattgaggatccagtttgttctgattatcccaattttaagattgtgtctatgctttaccagagcggagattacttactctccatattagggtctgatgggtataaaattattaagttcctcgaaccattgtgcttggccaaaattcaattatgctcaaagtacactgagaggaagggccgattcttaacacaaatgcatttagctgtaaatcacaccctagaagaaattacagaaatgcgtgcactaaagccttcacaggctcaaaagatccgtgaattccatagaacattgataaggctggagatgacgccacaacaactttgtgagctattttccattcaaaaacactgggggcatcctgtgctacatagtgaaacagcaatccaaaaagttaaaaaacatgctacggtgctaaaagcattacgccctatagtgattttcgagacatactgtgtttttaaatatagtattgccaaacattattttgatagtcaaggatcttggtacagtgttacttcagataggaatctaacaccgggtcttaattcttatatcaaaagaaatcaattccctccgttgccaatgattaaagaactactatgggaattttaccaccttgaccaccctccacttttctcaaccaaaattattagtgacttaagtatttttataaaagacagagctaccgcagtagaaaggacatgctgggatgcagtattcgagcctaatgttctaggatataatccacctcacaaatttagtactaaacgtgtaccggaacaatttttagagcaagaaaacttttctattgagaatgttctttcctacgcacaaaaactcgagtatctactaccacaatatcggaacttttctttctcattgaaagagaaagagttgaatgtaggtagaaccttcggaaaattgccttatccgactcgcaatgttcaaacactttgtgaagctctgttagctgatggtcttgctaaagcatttcctagcaatatgatggtagttacggaacgtgagcaaaaagaaagcttattgcatcaagcatcatggcaccacacaagtgatgattttggtgaacatgccacagttagagggagtagctttgtaactgatttagagaaatacaatcttgcatttagatatgagtttacagcaccttttatagaatattgcaaccgttgctatggtgttaagaatgtttttaattggatgcattatacaatcccacagtgttatatgcatgtcagtgattattataatccaccacataacctcacactggagaatcgagacaacccccccgaagggcctagttcatacaggggtcatatgggagggattgaaggactgcaacaaaaactctggacaagtatttcatgtgctcaaatttctttagttgaaattaagactggttttaagttacgctcagctgtgatgggtgacaatcagtgcattactgttttatcagtcttccccttagagactgacgcagacgagcaggaacagagcgccgaagacaatgcagcgagggtggccgccagcctagcaaaagttacaagtgcctgtggaatctttttaaaacctgatgaaacatttgtacattcaggttttatctattttggaaaaaaacaatatttgaatggggtccaattgcctcagtcccttaaaacggctacaagaatggcaccattgtctgatgcaatttttgatgatcttcaagggaccctggctagtataggcactgcttttgagcgatccatctctgagacacgacatatctttccttgcaggataaccgcagctttccatacgtttttttcggtgagaatcttgcaatatcatcatctcgggttcaataaaggttttgaccttggacagttaacactcggcaaacctctggatttcggaacaatatcattggcactagcggtaccgcaggtgcttggagggttatccttcttgaatcctgagaaatgtttctaccggaatctaggagatccagttacctcaggcttattccagttaaaaacttatctccgaatgattgagatggatgatttattcttacctttaattgcgaagaaccctgggaactgcactgccattgactttgtgctaaatcctagcggattaaatgtccctgggtcgcaagacttaacttcatttctgcgccagattgtacgcaggaccatcaccctaagtgcgaaaaacaaacttattaataccttatttcatgcgtcagctgacttcgaagacgaaatggtttgtaaatggctattatcatcaactcctgttatgagtcgttttgcggccgatatcttttcacgcacgccgagcgggaagcgattgcaaattctaggatacctggaaggaacacgcacattattagcctctaagatcatcaacaataatacagagacaccggttttggacagactgaggaaaataacattgcaaaggtggagcctatggtttagttatcttgatcattgtgataatatcctggcggaggctttaacccaaataacttgcacagttgatttagcacagattctgagggaatattcatgggctcatattttagagggaagacctcttattggagccacactcccatgtatgattgagcaattcaaagtgttttggctgaaaccctacgaacaatgtccgcagtgttcaaatgcaaagcaaccaggtgggaaaccattcgtgtcagtggcagtcaagaaacatattgttagtgcatggccgaacgcatcccgaataagctggactatcggggatggaatcccatacattggatcaaggacagaagataagataggacaacctgctattaaaccaaaatgtccttccgcagccttaagagaggccattgaattggcgtcccgtttaacatgggtaactcaaggcagttcgaacagtgacttgctaataaaaccatttttggaagcacgagtaaatttaagtgttcaagaaatacttcaaatgaccccttcacattactcaggaaatattgttcacaggtacaacgatcaatacagtcctcattctttcatggccaatcgtatgagtaattcagcaacgcgattgattgtttctacaaacactttaggtgagttttcaggaggtggccagtctgcacgcgacagcaatattattttccagaatgttataaattatgcagttgcactgttcgatattaaatttagaaacactgaggctacagatatccaatataatcgtgctcaccttcatctaactaagtgttgcacccgggaagtaccagctcagtatttaacatacacatctacattggatttagatttaacaagataccgagaaaacgaattgatttatgacagtaatcctctaaaaggaggactcaattgcaatatctcattcgataatccatttttccaaggtaaacggctgaacattatagaagatgatcttattcgactgcctcacttatctggatgggagctagccaagaccatcatgcaatcaattatttcagatagcaacaattcatctacagacccaattagcagtggagaaacaagatcattcactacccatttcttaacttatcccaagataggacttctgtacagttttggggcctttgtaagttattatcttggcaatacaattcttcggactaagaaattaacacttgacaattttttatattacttaactactcaaattcataatctaccacatcgctcattgcgaatacttaagccaacattcaaacatgcaagcgttatgtcacggttaatgagtattgatcctcatttttctatttacataggcggtgctgcaggtgacagaggactctcagatgcggccaggttatttttgagaacgtccatttcatcttttcttacatttgtaaaagaatggataattaatcgcggaacaattgtccctttatggatagtatatccgctagagggtcaaaacccaacacctgtgaataattttctctatcagatcgtagaactgctggtgcatgattcatcaagacaacaggcttttaaaactaccataagtgatcatgtacatcctcacgacaatcttgtttacacatgtaagagtacagccagcaatttcttccatgcatcattggcgtactggaggagcagacacagaaacagcaaccgaaaatacttggcaagagactcttcaactggatcaagcacaaacaacagtgatggtcatattgagagaagtcaagaacaaaccaccagagatccacatgatggcactgaacggaatctagtcctacaaatgagccatgaaataaaaagaacgacaattccacaagaaaacacgcaccagggtccgtcgttccagtcctttctaagtgactctgcttgtggtacagcaaatccaaaactaaatttcgatcgatcgagacacaatgtgaaatttcaggatcataactcggcatccaagagggaaggtcatcaaataatctcacaccgtctagtcctacctttctttacattatctcaagggacacgccaattaacgtcatccaatgagtcacaaacccaagacgagatatcaaagtacttacggcaattgagatccgtcattgataccacagtttattgtagatttaccggtatagtctcgtccatgcattacaaacttgatgaggtcctttgggaaatagagagtttcaagtcggctgtgacgctagcagagggagaaggtgctggtgccttactattgattcagaaataccaagttaagaccttatttttcaacacgctagctactgagtccagtatagagtcagaaatagtatcaggaatgactactcctaggatgcttctacctgttatgtcaaaattccataatgaccaaattgagattattcttaacaactcagcaagccaaataacagacataacaaatcctacttggtttaaagaccaaagagcaaggctacctaagcaagtcgaggttataaccatggatgcagagacaacagagaatataaacagatcgaaattgtacgaagctgtatataaattgatcttacaccatattgatcctagcgtattgaaagcagtggtccttaaagtctttctaagtgatactgagggtatgttatggctaaatgataatttagccccgttttttgccactggttatttaattaagccaataacgtcaagtgctagatctagtgagtggtatctttgtctgacgaacttcttatcaactacacgtaagatgccacaccaaaaccatctcagttgtaaacaggtaatacttacggcattgcaactgcaaattcaacgaagcccatactggctaagtcatttaactcagtatgctgactgtgagttacatttaagttatatccgccttggttttccatcattagagaaagtactataccacaggtataacctcgtcgattcaaaaagaggtccactagtctctatcactcagcacttagcacatcttagagcagagattcgagaattaactaatgattataatcaacagcgacaaagtcggactcaaacatatcactttattcgtactgcaaaaggacgaatcacaaaactagtcaatgattatttaaaattctttcttattgtgcaagcattaaaacataatgggacatggcaagctgagtttaagaaattaccagagttgattagtgtgtgcaataggttctaccatattagagattgcaattgtgaagaacgtttcttagttcaaaccttatatttacatagaatgcaggattctgaagttaagcttatcgaaaggctgacagggcttctgagtttatttccggatggtctctacaggtttgattgaattaccgtgcatagtatcctgatacttgcaaaggttggttattaacatacagattataaaaaactcataaattgctctcatacatcatattgatctaatctcaataaacaactatttaaataacgaaaggagtccctatattatatactatatttagcctctctccctgcgtgataatcaaaaaattcacaatgcagcatgtgtgacatattactgccgcaatgaatttaacgcaacataataaactctgcactctttataattaagctttaacgaaaggtctgggctcatattgttattgatataataatgttgtatcaatatcctgtcagatggaatagtgttttggttgataacacaacttcttaaaacaaaattgatctttaagattaagttttttataattatcattactttaatttgtcgttttaaaaacggtgatagccttaatctttgtgtaaaataagagattaggtgtaataaccttaacatttttgtctagtaagctactatttcatacagaatgataaaattaaaagaaaaggcaggactgtaaaatcagaaataccttctttacaatatagcagactagataataatcttcgtgttaatgataattaagacattgaccacgctcatcagaaggctcgccagaataaacgttgcaaaaaggattcctggaaaaatggtcgcacacaaaaatttaaaaataaatctatttcttcttttttgtgtgtcca

SEQ ID NO: 91 - DNA, 18797 bp, Ebola virus
gaataactatgaggaagattaataattttcctctcattgaaatttatatcggaatttaaattgaaattgtcactgtaatcacacctggtttgtctcagagccacatcacaaagatagagaacaacctaggtctccgaagggagcaagggcatcagtgtgctcagttgaaaatcccttgtcaacacctaggtcttatcacatcacaagttccacctcagactctgcagggtgatccaacaaccttaatagaaacattattgttaaaggacagcattagttcacagtcaaacaagcaagattgagaattaaccttggttttgaacttgaacacttaggggattgaagattcaacaaccctaaagcttggggtaaaacattggaaatagttaaaagacaaattgctcggaatcacaaaattccgagtatggattctcgtcctcagaaaatctggatggcgccgagtctcactgaatctgacatggattaccacaagatcttgacagcaggtctgtccgttcaacaggggattgttcggcaaagagtcatcccagtgtatcaagtaaacaatcttgaagaaatttgccaacttatcatacaggcctttgaagcaggtgttgattttcaagagagtgcggacagtttccttctcatgctttgtcttcatcatgcgtaccagggagattacaaacttttcttggaaagtggcgcagtcaagtatttggaagggcacgggttccgttttgaagtcaagaagcgtgatggagtgaagcgccttgaggaattgctgccagcagtatctagtggaaaaaacattaagagaacacttgctgccatgccggaagaggagacaactgaagctaatgccggtcagtttctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttctgatcaaatttctcctaatacaccaagggatgcacatggttgccgggcatgatgccaacgatgctgtgatttcaaattcagtggctcaagctcgtttttcaggcttattgattgtcaaaacagtacttgatcatatcctacaaaagacagaacgaggagttcgtctccatcctcttgcaaggaccgccaaggtaaaaaatgaggtgaactcctttaaggctgcactcagctccctggccaagcatggagagtatgctcctttcgcccgacttttgaacctttctggagtaaataatcttgagcatggtcttttccctcaactatcggcaattgcactcggagtcgccacagcacacgggagtaccctcgcaggagtaaatgttggagaacagtatcaacaactcagagaggctgccactgaggctgagaagcaactccaacaatatgcagagtctcgcgaacttgaccatcttggacttgatgatcaggaaaagaaaattcttatgaacttccatcagaaaaagaacgaaatcagcttccagcaaacaaacgctatggtaactctaagaaaagagcgcctggccaagctgacagaagctatcactgctgcgtcactgcccaaaacaagtggacattacgatgatgatgacgacattccctttccaggacccatcaatgatgacgacaatcctggccatcaagatgatgatccgactgactcacaggatacgaccattcccgatgtggtggttgatcccgatgatggaagctacggcgaataccagagttactcggaaaacggcatgaatgcaccagatgacttggtcctattcgatctagacgaggacgacgaggacactaagccagtgcctaatagatcgaccaagggtggacaacagaagaacagtcaaaagggccagcatatagagggcagacagacacaatccaggccaattcaaaatgtcccaggccctcacagaacaatccaccacgccagtgcgccactcacggacaatgacagaagaaatgaaccctccggctcaaccagccctcgcatgctgacaccaattaacgaagaggcagacccactggacgatgccgacgacgagacgtctagccttccgcccttggagtcagatgatgaagagcaggacagggacggaacttccaaccgcacacccactgtcgccccaccggctcccgtatacagagatcactctgaaaagaaagaactcccgcaagacgagcaacaagatcaggaccacactcaagaggccaggaaccaggacagtgacaacacccagtcagaacactcttttgaggagatgtatcgccacattctaagatcacaggggccatttgatgctgttttgtattatcatatgatgaaggatgagcctgtagttttcagtaccagtgatggcaaagagtacacgtatccagactcccttgaagaggaatatccaccatggctcactgaaaaagaggctatgaatgaagagaatagatttgttacattggatggtcaacaattttattggccggtgatgaatcacaagaataaattcatggcaatcctgcaacatcatcagtgaatgagcatggaacaatgggatgattcaaccgacaaatagctaacattaagtagtcaaggaacgaaaacaggaagaatttttgatgtctaaggtgtgaattattatcacaataaaagtgattcttatttttgaatttaaagctagcttattattactagccgtttttcaaagttcaatttgagtcttaatgcaaataggcgttaagccacagttatagccataattgtaactcaatattctaactagcgatttatctaaattaaattacattatgcttttataacttacctactagcctgcccaacatttacacgatcgttttataattaagaaaaaactaatgatgaagattaaaaccttcatcatccttacgtcaattgaattctctagcactcgaagcttattgtcttcaatgtaaaagaaaagctggtctaacaagatgacaactagaacaaagggcaggggccatactgcggccacgactcaaaacgacagaatgccaggccctgagctttcgggctggatctctgagcagctaatgaccggaagaattcctgtaagcgacatcttctgtgatattgagaacaatccaggattatgctacgcatcccaaatgcaacaaacgaagccaaacccgaagacgcgcaacagtcaaacccaaacggacccaatttgcaatcatagttttgaggaggtagtacaaacattggcttcattggctactgttgtgcaacaacaaaccatcgcatcagaatcattagaacaacgcattacgagtcttgagaatggtctaaagccagtttatgatatggcaaaaacaatctcctcattgaacagggtttgtgctgagatggttgcaaaatatgatcttctggtgatgacaaccggtcgggcaacagcaaccgctgcggcaactgaggcttattgggccgaacatggtcaaccaccacctggaccatcactttatgaagaaagtgcgattcggggtaagattgaatctagagatgagaccgtccctcaaagtgttagggaggcattcaacaatctaaacagtaccacttcactaactgaggaaaattttgggaaacctgacatttcggcaaaggatttgagaaacattatgtatgatcacttgcctggttttggaactgctttccaccaattagtacaagtgatttgtaaattgggaaaagatagcaactcattggacatcattcatgctgagttccaggccagcctggctgaaggagactctcctcaatgtgccctaattcaaattacaaaaagagttccaatcttccaagatgctgctccacctgtcatccacatccgctctcgaggtgacattccccgagcttgccagaaaagcttgcgtccagtcccaccatcgcccaagattgatcgaggttgggtatgtgtttttcagcttcaagatggtaaaacacttggactcaaaatttgagccaatctcccttccctccgaaagaggcgaataatagcagaggcttcaactgctgaactatagggtacgttacattaatgatacacttgtgagtatcagccctggataatataagtcaattaaacgaccaagataaaattgttcatatctcgctagcagcttaaaatataaatgtaataggagctatatctctgacagtattataatcaattgttattaagtaacccaaaccaaaagtgatgaagattaagaaaaacctacctcggctgagagagtgttttttcattaaccttcatcttgtaaacgttgagcaaaattgttaaaaatatgaggcgggttatattgcctactgctcctcctgaatatatggaggccatataccctgtcaggtcaaattcaacaattgctagaggtggcaacagcaatacaggcttcctgacaccggagtcagtcaatggggacactccatcgaatccactcaggccaattgccgatgacaccatcgaccatgccagccacacaccaggcagtgtgtcatcagcattcatccttgaagctatggtgaatgtcatatcgggccccaaagtgctaatgaagcaaattccaatttggcttcctctaggtgtcgctgatcaaaagacctacagctttgactcaactacggccgccatcatgcttgcttcatacactatcacccatttcggcaaggcaaccaatccacttgtcagagtcaatcggctgggtcctggaatcccggatcatcccctcaggctcctgcgaattggaaaccaggctttcctccaggagttcgttcttccgccagtccaactaccccagtatttcacctttgatttgacagcactcaaactgatcacccaaccactgcctgctgcaacatggaccgatgacactccaacaggatcaaatggagcgttgcgtccaggaatttcatttcatccaaaacttcgccccattcttttacccaacaaaagtgggaagaaggggaacagtgccgatctaacatctccggagaaaatccaagcaataatgacttcactccaggactttaagatcgttccaattgatccaaccaaaaatatcatgggaatcgaagtgccagaaactctggtccacaagctgaccggtaagaaggtgacttctaaaaatggacaaccaatcatccctgttcttttgccaaagtacattgggttggacccggtggctccaggagacctcaccatggtaatcacacaggattgtgacacgtgtcattctcctgcaagtcttccagctgtgattgagaagtaattgcaataattgactcagatccagttttatagaatcttctcagggatagtgataacatctatttagtaatccgtccattagaggagacacttttaattgatcaatatactaaaggtgctttacaccattgtcttttttctctcctaaatgtagaacttaacaaaagactcataatatacttgtttttaaaggattgattgatgaaagatcataactaataacattacaaataatcctactataatcaatacggtgattcaaatgttaatctttctcattgcacatactttttgcccttatcctcaaattgcctgcatgcttacatctgaggatagccagtgtgacttggattggaaatgtggagaaaaaatcgggacccatttctaggttgttcacaatccaagtacagacattgcccttctaattaagaaaaaatcggcgatgaagattaagccgacagtgagcgtaatcttcatctctcttagattatttgttttccagagtaggggtcgtcaggtccttttcaatcgtgtaaccaaaataaactccactagaaggatattgtggggcaacaacacaatgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttagtttttcttcagattgcttcatggaaaagctcagcctcaaatcaatgaaaccaggatttaattatatggattacttgaatctaagattacttgacaaatgataatataatacactggagctttaaacatagccaatgtgattctaactcctttaaactcacagttaatcataaacaaggtttgacatcaatctagttatctctttgagaatgataaacttgatgaagattaagaaaaaggtaatctttcgattatctttaatcttcatccttgattctacaatcatgacagttgtctttagtgacaagggaaagaagcctttttattaagttgtaataatcagatctgcgaaccggtagagtttagttgcaacctaacacacataaagcattggtcaaaaagtcaatagaaatttaaacagtgagtggagacaacttttaaatggaagcttcatatgagagaggacgcccacgagctgccagacagcattcaagggatggacacgaccaccatgttcgagcacgatcatcatccagagagaattatcgaggtgagtaccgtcaatcaaggagcgcctcacaagtgcgcgttcctactgtatttcataagaagagagttgaaccattaacagttcctccagcacctaaagacatatgtccgaccttgaaaaaaggatttttgtgtgacagtagtttttgcaaaaaagatcaccagttggagagtttaactgatagggaattactcctactaatcgcccgtaagacttgtggatcagtagaacaacaattaaatataactgcacccaaggactcgcgcttagcaaatccaacggctgatgatttccagcaagaggaaggtccaaaaattaccttgttgacactgatcaagacggcagaacactgggcgagacaagacatcagaaccatagaggattcaaaattaagagcattgttgactctatgtgctgtgatgacgaggaaattctcaaaatcccagctgagtcttttatgtgagacacacctaaggcgcgaggggcttgggcaagatcaggcagaacccgttctcgaagtatatcaacgattacacagtgataaaggaggcagttttgaagctgcactatggcaacaatgggaccgacaatccctaattatgtttatcactgcattcttgaatattgctctccagttaccgtgtgaaagttctgctgtcgttgtttcagggttaagaacattggttcctcaatcagataatgaggaagcttcaaccaacccggggacatgctcatggtctgatgagggtaccccttaataaggctgactaaaacactatataaccttctacttgatcacaatactccgtatacctatcatcatatatttaatcaagacgatatcctttaaaacttattcagtactataatcactctcgtttcaaattaataagatgtgcatgattgccctaatatatgaagaggtatgatacaaccctaacagtgatcaaagaaaatcataatctcgtatcgctcgtaatataacctgccaagcatacctcttgcacaaagtgattcttgtacacaaataatgttttactctacaggaggtagcaacgatccatcccatcaaaaaataagtatttcatgacttactaatgatctcttaaaatattaagaaaaactgacggaacataaattctttatgcttcaagctgtggaggaggtgtttggtattggctattgttatattacaatcaataacaagcttgtaaaaatattgttcttgtttcaagaggtagattgtgaccggaaatgctaaactaatgatgaagattaatgcggaggtctgataagaataaaccttattattcagattaggccccaagaggcattcttcatctccttttagcaaagtactatttcagggtagtccaattagtggcacgtcttttagctgtatatcagtcgcccctgagatacgccacaaaagtgtctctaagctaaattggtctgtacacatcccatacattgtattaggggcaataatatctaattgaacttagccgtttaaaatttagtgcataaatctgggctaacaccaccaggtcaactccattggctgaaaagaagcttacctacaacgaacatcactttgagcgccctcacaattaaaaaataggaacgtcgttccaacaatcgagcgcaaggtttcaaggttgaactgagagtgtctagacaacaaaatattgatactccagacaccaagcaagacctgagaaaaaaccatggctaaagctacgggacgatacaatctaatatcgcccaaaaaggacctggagaaaggggttgtcttaagcgacttctgtaacttcttagttagccaaactattcaggggtggaaggtttattgggctggtattgagtttgatgtgactcacaaaggaatggccctattgcatagactgaaaactaatgactttgcccctgcatggtcaatgacaaggaatctctttcctcatttatttcaaaatccgaattccacaattgaatcaccgctgtgggcattgagagtcatccttgcagcagggatacaggaccagctgattgaccagtctttgattgaacccttagcaggagcccttggtctgatctctgattggctgctaacaaccaacactaaccatttcaacatgcgaacacaacgtgtcaaggaacaattgagcctaaaaatgctgtcgttgattcgatccaatattctcaagtttattaacaaattggatgctctacatgtcgtgaactacaacggattgttgagcagtattgaaattggaactcaaaatcatacaatcatcataactcgaactaacatgggttttctggtggagctccaagaacccgacaaatcggcaatgaaccgcatgaagcctgggccggcgaaattttccctccttcatgagtccacactgaaagcatttacacaaggatcctcgacacgaatgcaaagtttgattcttgaatttaatagctctcttgctatctaactaaggtagaatacttcatattgagctaactcatatatgctgactcaatagttatcttgacatctctgctttcataatcagatatataagcataataaataaatactcatatttcttgataatttgtttaaccacagataaatcctcactgtaagccagcttccaagttgacacccttacaaaaaccaggactcagaatccctcaaacaagagattccaagacaacatcatagaattgctttattatatgaataagcattttatcaccagaaatcctatatactaaatggttaattgtaactgaacccgcaggtcacatgtgttaggtttcacagattctatatattactaactctatactcgtaattaacattagataagtagattaagaaaaaagcctgaggaagattaagaaaaactgcttattgggtctttccgtgttttagatgaagcagttgaaattcttcctcttgatattaaatggctacacaacatacccaatacccagacgctaggttatcatcaccaattgtattggaccaatgtgacctagtcactagagcttgcgggttatattcatcatactcccttaatccgcaactacgcaactgtaaactcccgaaacatatctaccgtttgaaatacgatgtaactgttaccaagttcttgagtgatgtaccagtggcgacattgcccatagatttcatagtcccagttcttctcaaggcactgtcaggcaatggattctgtcctgttgagccgcggtgccaacagttcttagatgaaatcattaagtacacaatgcaagatgctctcttcttgaaatattatctcaaaaatgtgggtgctcaagaagactgtgttgatgaacactttcaagagaaaatcttatcttcaattcagggcaatgaatttttacatcaaatgtttttctggtatgatctggctattttaactcgaaggggtagattaaatcgaggaaactctagatcaacatggtttgttcatgatgatttaatagacatcttaggctatggggactatgttttttggaagatcccaatttcaatgttaccactgaacacacaaggaatcccccatgctgctatggactggtatcaggcatcagtattcaaagaagcggttcaagggcatacacacattgtttctgtttctactgccgacgtcttgataatgtgcaaagatttaattacatgtcgattcaacacaactctaatctcaaaaatagcagagattgaggatccagtttgttctgattatcccaattttaagattgtgtctatgctttaccagagcggagattacttactctccatattagggtctgatgggtataaaattattaagttcctcgaaccattgtgcttggccaaaattcaattatgctcaaagtacactgagaggaagggccgattcttaacacaaatgcatttagctgtaaatcacaccctagaagaaattacagaaatgcgtgcactaaagccttcacaggctcaaaagatccgtgaattccatagaacattgataaggctggagatgacgccacaacaactttgtgagctattttccattcaaaaacactgggggcatcctgtgctacatagtgaaacagcaatccaaaaagttaaaaaacatgctacggtgctaaaagcattacgccctatagtgattttcgagacatactgtgtttttaaatatagtattgccaaacattattttgatagtcaaggatcttggtacagtgttacttcagataggaatctaacaccgggtcttaattcttatatcaaaagaaatcaattccctccgttgccaatgattaaagaactactatgggaattttaccaccttgaccaccctccacttttctcaaccaaaattattagtgacttaagtatttttataaaagacagagctaccgcagtagaaaggacatgctgggatgcagtattcgagcctaatgttctaggatataatccacctcacaaatttagtactaaacgtgtaccggaacaatttttagagcaagaaaacttttctattgagaatgttctttcctacgcacaaaaactcgagtatctactaccacaatatcggaacttttctttctcattgaaagagaaagagttgaatgtaggtagaaccttcggaaaattgccttatccgactcgcaatgttcaaacactttgtgaagctctgttagctgatggtcttgctaaagcatttcctagcaatatgatggtagttacggaacgtgagcaaaaagaaagcttattgcatcaagcatcatggcaccacacaagtgatgattttggtgaacatgccacagttagagggagtagctttgtaactgatttagagaaatacaatcttgcatttagatatgagtttacagcaccttttatagaatattgcaaccgttgctatggtgttaagaatgtttttaattggatgcattatacaatcccacagtgttatatgcatgtcagtgattattataatccaccacataacctcacactggagaatcgagacaacccccccgaagggcctagttcatacaggggtcatatgggagggattgaaggactgcaacaaaaactctggacaagtatttcatgtgctcaaatttctttagttgaaattaagactggttttaagttacgctcagctgtgatgggtgacaatcagtgcattactgttttatcagtcttccccttagagactgacgcagacgagcaggaacagagcgccgaagacaatgcagcgagggtggccgccagcctagcaaaagttacaagtgcctgtggaatctttttaaaacctgatgaaacatttgtacattcaggttttatctattttggaaaaaaacaatatttgaatggggtccaattgcctcagtcccttaaaacggctacaagaatggcaccattgtctgatgcaatttttgatgatcttcaagggaccctggctagtataggcactgcttttgagcgatccatctctgagacacgacatatctttccttgcaggataaccgcagctttccatacgtttttttcggtgagaatcttgcaatatcatcatctcgggttcaataaaggttttgaccttggacagttaacactcggcaaacctctggatttcggaacaatatcattggcactagcggtaccgcaggtgcttggagggttatccttcttgaatcctgagaaatgtttctaccggaatctaggagatccagttacctcaggcttattccagttaaaaacttatctccgaatgattgagatggatgatttattcttacctttaattgcgaagaaccctgggaactgcactgccattgactttgtgctaaatcctagcggattaaatgtccctgggtcgcaagacttaacttcatttctgcgccagattgtacgcaggaccatcaccctaagtgcgaaaaacaaacttattaataccttatttcatgcgtcagctgacttcgaagacgaaatggtttgtaaatggctattatcatcaactcctgttatgagtcgttttgcggccgatatcttttcacgcacgccgagcgggaagcgattgcaaattctaggatacctggaaggaacacgcacattattagcctctaagatcatcaacaataatacagagacaccggttttggacagactgaggaaaataacattgcaaaggtggagcctatggtttagttatcttgatcattgtgataatatcctggcggaggctttaacccaaataacttgcacagttgatttagcacagattctgagggaatattcatgggctcatattttagagggaagacctcttattggagccacactcccatgtatgattgagcaattcaaagtgttttggctgaaaccctacgaacaatgtccgcagtgttcaaatgcaaagcaaccaggtgggaaaccattcgtgtcagtggcagtcaagaaacatattgttagtgcatggccgaacgcatcccgaataagctggactatcggggatggaatcccatacattggatcaaggacagaagataagataggacaacctgctattaaaccaaaatgtccttccgcagccttaagagaggccattgaattggcgtcccgtttaacatgggtaactcaaggcagttcgaacagtgacttgctaataaaaccatttttggaagcacgagtaaatttaagtgttcaagaaatacttcaaatgaccccttcacattactcaggaaatattgttcacaggtacaacgatcaatacagtcctcattctttcatggccaatcgtatgagtaattcagcaacgcgattgattgtttctacaaacactttaggtgagttttcaggaggtggccagtctgcacgcgacagcaatattattttccagaatgttataaattatgcagttgcactgttcgatattaaatttagaaacactgaggctacagatatccaatataatcgtgctcaccttcatctaactaagtgttgcacccgggaagtaccagctcagtatttaacatacacatctacattggatttagatttaacaagataccgagaaaacgaattgatttatgacagtaatcctctaaaaggaggactcaattgcaatatctcattcgataatccatttttccaaggtaaacggctgaacattatagaagatgatcttattcgactgcctcacttatctggatgggagctagccaagaccatcatgcaatcaattatttcagatagcaacaattcatctacagacccaattagcagtggagaaacaagatcattcactacccatttcttaacttatcccaagataggacttctgtacagttttggggcctttgtaagttattatcttggcaatacaattcttcggactaagaaattaacacttgacaattttttatattacttaactactcaaattcataatctaccacatcgctcattgcgaatacttaagccaacattcaaacatgcaagcgttatgtcacggttaatgagtattgatcctcatttttctatttacataggcggtgctgcaggtgacagaggactctcagatgcggccaggttatttttgagaacgtccatttcatcttttcttacatttgtaaaagaatggataattaatcgcggaacaattgtccctttatggatagtatatccgctagagggtcaaaacccaacacctgtgaataattttctctatcagatcgtagaactgctggtgcatgattcatcaagacaacaggcttttaaaactaccataagtgatcatgtacatcctcacgacaatcttgtttacacatgtaagagtacagccagcaatttcttccatgcatcattggcgtactggaggagcagacacagaaacagcaaccgaaaatacttggcaagagactcttcaactggatcaagcacaaacaacagtgatggtcatattgagagaagtcaagaacaaaccaccagagatccacatgatggcactgaacggaatctagtcctacaaatgagccatgaaataaaaagaacgacaattccacaagaaaacacgcaccagggtccgtcgttccagtcctttctaagtgactctgcttgtggtacagcaaatccaaaactaaatttcgatcgatcgagacacaatgtgaaatttcaggatcataactcggcatccaagagggaaggtcatcaaataatctcacaccgtctagtcctacctttctttacattatctcaagggacacgccaattaacgtcatccaatgagtcacaaacccaagacgagatatcaaagtacttacggcaattgagatccgtcattgataccacagtttattgtagatttaccggtatagtctcgtccatgcattacaaacttgatgaggtcctttgggaaatagagagtttcaagtcggctgtgacgctagcagagggagaaggtgctggtgccttactattgattcagaaataccaagttaagaccttatttttcaacacgctagctactgagtccagtatagagtcagaaatagtatcaggaatgactactcctaggatgcttctacctgttatgtcaaaattccataatgaccaaattgagattattcttaacaactcagcaagccaaataacagacataacaaatcctacttggtttaaagaccaaagagcaaggctacctaagcaagtcgaggttataaccatggatgcagagacaacagagaatataaacagatcgaaattgtacgaagctgtatataaattgatcttacaccatattgatcctagcgtattgaaagcagtggtccttaaagtctttctaagtgatactgagggtatgttatggctaaatgataatttagccccgttttttgccactggttatttaattaagccaataacgtcaagtgctagatctagtgagtggtatctttgtctgacgaacttcttatcaactacacgtaagatgccacaccaaaaccatctcagttgtaaacaggtaatacttacggcattgcaactgcaaattcaacgaagcccatactggctaagtcatttaactcagtatgctgactgtgagttacatttaagttatatccgccttggttttccatcattagagaaagtactataccacaggtataacctcgtcgattcaaaaagaggtccactagtctctatcactcagcacttagcacatcttagagcagagattcgagaattaactaatgattataatcaacagcgacaaagtcggactcaaacatatcactttattcgtactgcaaaaggacgaatcacaaaactagtcaatgattatttaaaattctttcttattgtgcaagcattaaaacataatgggacatggcaagctgagtttaagaaattaccagagttgattagtgtgtgcaataggttctaccatattagagattgcaattgtgaagaacgtttcttagttcaaaccttatatttacatagaatgcaggattctgaagttaagcttatcgaaaggctgacagggcttctgagtttatttccggatggtctctacaggtttgattgaattaccgtgcatagtatcctgatacttgcaaaggttggttattaacatacagattataaaaaactcataaattgctctcatacatcatattgatctaatctcaataaacaactatttaaataacgaaaggagtccctatattatatactatatttagcctctctccctgcgtgataatcaaaaaattcacaatgcagcatgtgtgacatattactgccgcaatgaatttaacgcaacataataaactctgcactctttataattaagctttaacgaaaggtctgggctcatattgttattgatataataatgttgtatcaatatcctgtcagatggaatagtgttttggttgataacacaacttcttaaaacaaaattgatctttaagattaagttttttataattatcattactttaatttgtcgttttaaaaacggtgatagccttaatctttgtgtaaaataagagattaggtgtaataaccttaacatttttgtctagtaagctactatttcatacagaatgataaaattaaaagaaaaggcaggactgtaaaatcagaaataccttctttacaatatagcagactagataataatcttcgtgttaatgataattaaga

SEQ ID NO: 92 - DNA, 18958 bp, Ebola virus
cggacacacaaaaagaaagaagaatttttaggatcttttgtgtgcgaataactatgaggaagattaataattttcctctcattgaaatttatatcggaatttaaattgaaattgttactgtaatcatacctggtttgtttcagagccatatcaccaagatagagaacaacctaggtctccggagggggcaagggcatcagtgtgctcagttgaaaatcccttgtcaacatctaggccttatcacatcacaagttccgccttaaactctgcagggtgatccaacaaccttaatagcaacattattgttaaaggacagcattagttcacagtcaaacaagcaagattgagaattaactttgattttgaacctgaacacccagaggactggagactcaacaaccctaaagcctggggtaaaacattagaaatagtttaaagacaaattgctcggaatcacaaaattccgagtatggattctcgtcctcagaaagtctggatgacgccgagtctcactgaatctgacatggattaccacaagatcttgacagcaggtctgtccgttcaacaggggattgttcggcaaagagtcatcccagtgtatcaagtaaacaatcttgaggaaatttgccaacttatcatacaggcctttgaagctggtgttgattttcaagagagtgcggacagtttccttctcatgctttgtcttcatcatgcgtaccaaggagattacaaacttttcttggaaagtggcgcagtcaagtatttggaagggcacgggttccgttttgaagtcaagaagtgtgatggagtgaagcgccttgaggaattgctgccagcagtatctagtgggagaaacattaagagaacacttgctgccatgccggaagaggagacgactgaagctaatgccggtcagttcctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttttgatcaaatttcttctaatacaccaagggatgcacatggttgccggacatgatgccaacgatgctgtgatttcaaattcagtggctcaagctcgtttttcaggtctattgattgtcaaaacagtacttgatcatatcctacaaaagacagaacgaggagttcgtctccatcctcttgcaaggaccgccaaggtaaaaaatgaggtgaactccttcaaggctgcactcagctccctggccaagcatggagagtatgctcctttcgcccgacttttgaacctttctggagtaaataatcttgagcatggtcttttccctcaactgtcggcaattgcactcggagtcgccacagcccacgggagcaccctcgcaggagtaaatgttggagaacagtatcaacagctcagagaggcagccactgaggctgagaagcaactccaacaatatgcggagtctcgtgaacttgaccatcttggacttgatgatcaggaaaagaaaattcttatgaacttccatcagaaaaagaacgaaatcagcttccagcaaacaaacgcgatggtaactctaagaaaagagcgcctggccaagctgacagaagctatcactgctgcatcactgcccaaaacaagtggacattacgatgatgatgacgacattccctttccaggacccatcaatgatgacgacaatcctggccatcaagatgatgatccgactgactcacaggatacgaccattcccgatgtggtagttgaccccgatgatggaggctacggcgaataccaaagttactcggaaaacggcatgagtgcaccagatgacttggtcctattcgatctagacgaggacgacgaggacaccaagccagtgcctaacagatcgaccaagggtggacaacagaaaaacagtcaaaagggccagcatacagagggcagacagacacaatccacgccaactcaaaacgtcacaggccctcgcagaacaatccaccatgccagtgctccactcacggacaatgacagaagaaacgaaccctccggctcaaccagccctcgcatgctgaccccaatcaacgaagaggcagacccactggacgatgccgacgacgagacgtctagccttccgcccttagagtcagatgatgaagaacaggacagggacggaacttctaaccgcacacccactgtcgccccaccggctcccgtatacagagatcactccgaaaagaaagaactcccgcaagatgaacaacaagatcaggaccacattcaagaggccaggaaccaagacagtgacaacacccagccagaacattcttttgaggagatgtatcgccacattctaagatcacaggggccatttgatgccgttttgtattatcatatgatgaaggatgagcctgtagttttcagtaccagtgatggtaaagagtacacgtatccggactcccttgaagaggaatatccaccatggctcactgaaaaagaggccatgaatgatgagaatagatttgttacactggatggtcaacaattttattggccagtaatgaatcacaggaataaattcatggcaatcctgcaacatcatcagtgaatgagcatgtaataatgggatgatttaatcgacaaatagctaacattaaatagtcaaggaacgcaaacaggaagaatttttgatgtctaaggtgtgaattattatcacaataaaagtgattcttagttttgaatttaaagctagcttattattactagccgtttttcaaagttcaatttgagtcttaatgcaaataagcgttaagccacagttatagccataatggtaactcaatatcttagccagcgatttatctaaattaaattacattatgcttttataacttacctactagcctgcccaacatttacacgatcgttttataattaagaaaaaactaatgatgaagattaaaaccttcatcatccttacgtcaattgaattctctagcactagaagcttattgtcttcaatgtaaaagaaaagctggcctaacaagatgacaactagaacaaagggcaggggccatactgtggccacgactcaaaacgacagaatgccaggccctgagctttcgggctggatctctgagcagctaatgaccggaaggattcctgtaaacgacatcttctgtgatattgagaacaatccaggattatgctacgcatcccaaatgcaacaaacgaagccaaacccgaagatgcgcaacagtcaaacccaaacggacccaatttgcaatcatagttttgaggaggtagtacaaacattggcttcattggctactgttgtgcaacaacaaaccatcgcatcagaatcattagaacaacgcattacgagtcttgagaatggtctaaagccagtttatgatatggcaaaaacaatctcctcattgaacagggtttgtgctgagatggttgcaaaatatgatcttctggtgatgacaaccggtcgggcaacagcaaccgctgcggcaactgaggcttattgggctgaacatggtcaaccaccacctggaccatcactttatgaagaaagtgcgattcggggtaagattgaatctagagatgagactgtccctcaaagtgttagggaggcattcaacaatctagacagtaccacttcactaactgaggaaaattttgggaaacctgacatttcggcaaaggatttgagaaacattatgtatgatcacttgcctggttttggaactgctttccaccaattagtacaagtgatttgtaaattgggaaaagatagcaattcattggacattattcatgctgagttccaggccagcctggctgaaggagactcccctcaatgtgccctaattcaaattacaaaaagagttccaatcttccaagatgctgctccacctgtcatccacatccgctctcgaggtgacattccccgagcttgccagaagagcttgcgtccagtcccaccatcacccaagattgatcgaggttgggtatgtgtttttcagcttcaagatggtaaaacacttggactcaaaatttgagccaatctcttttccctccgaaagaggcaactaatagcagaggcttcaactgctgaactatagggtatgttacattaatgatacacttgtgagtatcagccctagataatataagtcaattaaacaaccaagataaaattgttcatatcccgctagcagctttaaagataaatgtaataggagctatacctctgacagtattataattaattgttattaagtaacccaaaccaaaaatgatgaagattaagaaaaacctacctcgactgagagagtgttttttcattaaccttcatcttgtaaacgttgagcaaaattgttaaaaatatgaggcgggttatattgcctactgctcctcctgaatatatggaggccatataccctgccaggtcaaattcaacaattgctaggggtggcaacagcaatacaggcttcctgacaccggagtcagtcaatggagacactccatcgaatccactcaggccaattgctgatgacaccattgaccatgccagccacacaccaggcagtgtgtcatcagcattcatcctcgaagctatggtgaatgtcatatcgggccccaaagtgctaatgaagcaaattccaatttggcttcctctaggtgtcgctgatcaaaagacctacagctttgactcaactacggccgccatcatgcttgcttcatatactatcacccatttcggcaaggcaaccaatccgcttgtcagagtcaatcggctgggtcctggaatcccggatcaccccctcaggctcctgcgaattggaaaccaggctttcctccaggagttcgttcttccaccagtccaactaccccagtatttcacctttgatttgacagcactcaaactgatcactcaaccactgcctgctgcaacatggaccgatgacactccaactggatcaaatggagcgttgcgtccaggaatttcatttcatccaaaacttcgccccattcttttacccaacaaaagtgggaagaaggggaacagtgccgatctaacatctccggagaaaatccaagcaataatgacttcactccaggactttaagatcgttccaattgatccaaccaaaaatatcatgggtatcgaagtgccagaaactctggtccacaagctgaccggtaagaaggtgacttccaaaaatggacaaccaatcatccctgttcttttgccaaagtacattgggttggacccggtggctccaggagacctcaccatggtaatcacacaggattgtgacacgtgtcattctcctgcaagtcttccagctgtggttgagaagtaattgcaataattgactcagatccagttttacagaatcttctcagggatagtgataacatctttttaataatccgtctactagaagagatacttctaattgatcaatatactaaaggtgctttacaccattgtctcttttctctcctaaatgtagagcttaacaaaagactcataatatacctgtttttaaaagattgattgatgaaagatcatgactaataacattacaaacaatcctactataatcaatacggtgattcaaatgtcaatctttctcattgcacatactctttgtccttatcctcaaattgcctacatgcttacatctgaggacagccagtgtgacttggattggagatgtggaggaaaaatcggggcccatttctaagttgttcacaatctaagtacagacattgctcttctaattaagaaaaaatcggcgatgaagattaagccgacagtgagcgtaatcttcatctctcttagattatttgtcttccagagtaggggtcatcaggtccttttcaattggataaccaaaataagcttcactagaaggatattgtgaggcgacaacacaatgggtgttacaggaatattgcagttacctcgtgatcgattcaagaggacatcattctttctttgggtaattatccttttccaaagaacattttccatcccgcttggagttatccacaatagtacattacaggttagtgatgtcgacaaactagtttgtcgtgacaaactgtcatccacaaatcaattgagatcagttggactgaatctcgaggggaatggagtggcaactgacgtgccatctgtgactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccatgtgccggagactttgccttccacaaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtcgagtggctattattctaccacaattagatatcaggctaccggttttggaactaatgagacagagtacttgttcgaggttgacaatttgacctacgtccaacttgaatcaagattcacaccacagtttctgctccagctgaatgagacaatatatgcaagtgggaagaggagcaacaccacgggaaaactaatttggaaggtcaaccccgaaattgatacaacaatcggggagtgggccttctgggaaactaaaaaaacctcactagaaaaattcgcagtgaagagttgtctttcacagctgtatcaaacggacccaaaaacatcagtggtcagagtccggcgcgaacttcttccgacccagagaccaacacaacaaatgaagaccacaaaatcatggcttcagaaaattcctctgcaatggttcaagtgcacagtcaaggaaggaaagctgcagtgtcgcatctgacaacccttgccacaatctccacgagtcctcaacctcccacaaccaaaacaggtccggacaacagcacccataatacacccgtgtataaacttgacatctctgaggcaactcaagttggacaacatcaccgtagagcagacaacgacagcacagcctccgacactccccccgccacgaccgcagccggacccttaaaagcagagaacaccaacacgagtaagagcgctgactccctggacctcgccaccacgacaagcccccaaaactacagcgagactgctggcaacaacaacactcatcaccaagataccggagaagagagtgccagcagcgggaagctaggcttaattaccaatactattgctggagtagcaggactgatcacaggcgggagaaggactcgaagagaagtaattgtcaatgctcaacccaaatgcaaccccaatttacattactggactactcaggatgaaggtgctgcaatcggattggcctggataccatatttcgggccagcagccgagggaatttacacagaggggctaatgcacaaccaagatggtttaatctgtgggttgaggcagctggccaacgaaacgactcaagctctccaactgttcctgagagccacaactgagctgcgaaccttttcaatcctcaaccgtaaggcaattgacttcctgctgcagcgatggggtggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggtgttataattgcagttatcgctttattctgtatatgcaaatttgtcttttagtctttcttcagattgtttcacggcaaaactcaacctcaaatcaatgaaactaggatttaattatatgaatcacttgaatctaagattacttgacaaatgataacataatacactggagcttcaaacatagccaatgtgattctaactcctttaaactcacagttaatcataaacaaggtttgacatcaatctagctatatctttaagaatgataaacttgatgaagattaagaaaaaggtaatctttcgattatctttagtcttcatccttgattctacaatcatgacagttgtctttaatgaaaaaggaaaaaagcctttttattaagttgtaataatcagatctgcaaaccggtagaatttagttgtaacctaacacacacaaagcattggtaaaaaagtcaatagaaatttaaacagtgagtgcagacaactcttaaatggaagcttcatatgagagaggacgcccccgagctgccagacagcattcaagggatggacacgaccaccatgttcgagcacgatcatcatccagagagaattatcgaggtgagtaccgtcaatcaaggagcgcctcacaagtgcgcgttcctactgtatttcataagaagagagttgaaccattaacagttcctccagcacctaaagacatatgtccgaccttgaaaaaaggatttttgtgtgacagtagtttttgcaaaaaagaccaccagttagaaagtttaactgatagggaattactcctactaatcgcccgtaagacttgtggatcagtagaacaacaattaaatataactgcacccaaggactcgcgcttagcaaatccaacggctgatgatttccagcaagaggaaggtcccaaaattaccttgttgacactgatcaagacggcagaacactgggcgagacaagacatccgaaccatagaggattccaaattaagggcattgttaactctatgtgctgtgatgacgaggaaattctcaaaatcccagctgagtcttttgtgtgagacacacctaaggcgcgaagggcttgggcaagatcaggcagaacccgttctcgaagtatatcaacgattacacagtgataaaggaggcagttttgaagctgcactatggcaacaatgggaccgacaatccctaattatgtttatcactgcattcttgaatatcgctctccagttaccgtgtgaaagttctgctgtcgttgtttcagggttaagaacattggttcctcaatcagataatgaggaagcttcaaccaacccggggacatgctcatggtctgatgagggtaccccttaataaggctgactaaaacactatataaccttctacttgatcacaatactccgtatacctatcatcatatatttaatcaagacgatatcctttaaaacttattcagtactataatcactctcatttcaaattgataagatatgcataattgccttaatatataaagaggtatgatataacccaaacattgaccaaagaaaatcataatctcgtatcgctcgcaatataacctgccaagcatacctcttgcacaaagtgattcttgtacacaaataatgtttgactctacaggaggtagcaacgatccatctcatcaaaaaataagtattttatgatttactaatgatctcttaaaatattaagaaaaactgacggaacataaattctttctgcttcaagttgtggaggaggtctatggtattcgctattgttatattacaatcaataacaagcttgtaaaaatattgttcttgtttcaggaggtatattgtgaccggaaaagctaaactaatgatgaagattaatgcggaggtctgatgagaataaaccttattattcagattaggccccaagaggcattcttcatctccttttagcaaaatactatttcaggatagtccagctagtgacacgtcttttagctgtataccagttgcccctgagatacgccacaaaagtgtctctgagctaaagtggtctgtacacatctcatacattgtattaggggcaataatatctaattgaacttagccatttaaaatttagtgcataaatctgggctaactccaccaggtcaactccattggctgaaaagaagcccacctacaacgaacattactttgagcgccctcacaattaaaaaataagagcgtcgttccaacaatcgagcgcaaggttacaaggttgaactgagagtgtctagacaacaaaatatcgatactccagacaccaagcaagacctgagaaaaaaccatggccaaagctacgggacgatacaatctaatatcgcccaaaaaggacctggagaaaggggttgtcttaagcgacctctgtaacttcttagttagtcaaactattcaagggtggaaagtttattgggctggtattgagtttgatgtgactcacaaaggaatggccctattgcatagactgaaaactaatgactttgcccctgcatggtcaatgacaaggaacctatttccccatttatttcaaaatccgaattccactattgaatcaccgctgtgggcactgagagtcatccttgcagcagggatacaggaccagttaattgaccagtctttgattgaacccttagcaggagcccttggtctgatctctgattggctgctaacaaccaacactaaccatttcaacatgcgaacacaacgtgtcaaggaacaattgagcctaaaaatgctgtcgttgattcgatccaatattctcaagtttattaacaaattggatgctctacatgtcgtgaactacaatggattattgagcagtattgaaattggaactcaaaatcatacaatcatcataactcgaactaacatgggttttctggtggagctccaagaacccgacaaatcggcaatgaaccgcaagaagcctgggccggcgaaattttccctccttcatgagtccacactgaaagcatttacacaagggtcctcgacacgaatgcaaagtttaattcttgaattcaatagctctcttgctatctaactaagatggaatacttcatattgggctaactcatatatgctgactcaatagttaacttgacatctctgccttcataatcagatatataagcataataaataaatactcatatttcttgataatttgtttaaccacagataaatcctcactgtaagccagcttccaagttgacacccttacaaaaaccaggactcagaatccctcaaataagagattccaagacaacatcatagaattgctttattatattaataagcattttatcactagaaatccaatatacgaaatggttaattgtaactaaacccgcaggtcatgtgtgttaggtttcacaaattatatatattactaactccatactcgtaactaacattagataagtaggttaagaaaaaagcttgaggaagattaagaaaaactgcttattgggtctttccgtgttttagatgaagcagttgacattcttcctcttgatattaaatggctacacaacatacccaatacccagacgccaggttatcatcaccaattgtattggaccaatgtgaccttgtcactagagcttgcgggttgtattcatcatactcccttaatccgcaactacgcaactgtaaactcccgaaacatatataccgtttaaaatatgatgtaactgttaccaagttcttaagtgatgtaccagtggcgacattgcccatagatttcatagtcccaattcttctcaaggcactatcaggcaatgggttctgtcctgttgagccgcggtgccaacagttcttagatgaaattattaagtacacaatgcaagatgctctcttcctgaaatattatctcaaaaatgtgggtgctcaagaagactgtgttgatgaccactttcaagaaaaaatcttatcttcaattcagggcaatgaatttttacatcaaatgtttttctggtatgacctggctattttaactcgaaggggtagattaaatcgaggaaactctagatcaacgtggtttgttcatgatgatttaatagacatcttaggctatggggactatgttttttggaagatcccaatttcactgttaccactgaacacacaaggaatcccccatgctgctatggattggtatcagacatcagtattcaaagaagcggttcaagggcatacacacattgtttctgtttctactgccgatgtcttgataatgtgcaaagatttaattacatgtcgattcaacacaactctaatctcaaaaatagcagaggttgaggacccagtttgctctgattatcccaattttaagattgtgtctatgctttaccagagcggagattacttactctccatattagggtctgatgggtataaaatcattaagtttctcgaaccattgtgcttggctaaaattcaattgtgctcaaagtacaccgagaggaagggccgattcttaacacaaatgcatttagctgtaaatcacaccctggaagaaattacagaaatacgtgcactaaagccttcacaggctcacaagatccgtgaattccatagaacattgataaggctggagatgacgccacaacaactttgtgagctattttccatacaaaaacactgggggcatcctgtgctacatagtgaaacagcaatccaaaaagttaaaaaacatgctacggtgctaaaagcattacgccctatcgtgattttcgagacatattgtgtttttaaatatagcattgcaaaacattattttgatagtcaaggatcttggtacagtgttacctcagatagaaatctaacaccaggtcttaattcttatatcaaaagaaatcaattccctccgttgccaatgattaaagaactgctatgggaattttaccaccttgaccatcctccacttttctcaaccaaaattattagtgacttaagtatttttataaaagacagagctactgcagtagaaaggacatgctgggatgcagtattcgagcctaatgttctgggatataatccacctcacaaattcagtaccaaacgtgtaccggaacaatttttagagcaagaaaacttttctattgagaatgttctttcctacgcgcaaaaactcgagtatctactaccacaatatcggaatttttctttctcattgaaagagaaagagttgaatgtaggtagaactttcggaaaattgccttatccgactcgcaatgttcaaacactttgtgaagctctgttagctgatggtcttgctaaagcatttcctagcaatatgatggtagttacggaacgtgaacaaaaagaaagcttattgcatcaagcatcatggcaccacacaagtgatgatttcggtgagcatgccacagttagagggagtagctttgtaactgatttagagaaatacaatcttgcatttaggtatgagtttacagcaccttttatagaatattgcaaccgttgctatggtgttaagaatgtttttaattggatgcattatacaatcccacagtgttatatgcatgtcagtgattattataatccaccgcataacctcacactggaaaatcgaaacaacccccctgaagggcctagttcatacaggggtcatatgggagggattgaaggactgcaacaaaaactctggacaagtatttcatgtgctcaaatttctttagttgaaattaagactggttttaagttgcgctcagctgtgatgggtgacaatcagtgcattaccgttttatcagtcttccccttagagactgatgcaggcgagcaggaacagagcgccgaggacaatgcagcgagggtggccgccagcctagcaaaagttacaagtgcctgtggaatctttttaaaacctgatgaaacatttgtacattcaggttttatctattttggaaaaaaacaatatttgaatggggtccaattgcctcagtcccttaaaacggctacaagaatggcaccattgtctgatgcaatttttgatgatcttcaagggaccctggctagtataggtactgcttttgagcgatccatctctgagacacgacatatctttccttgcagaataaccgcagctttccatacgttcttttcggtgagaatcttgcaatatcatcacctcggatttaataaaggttttgaccttggacagttaacactcggcaaacctctggatttcggaacaatatcattggcactagcggtaccgcaggtgcttggagggttatccttcttgaatcctgagaaatgtttctaccggaatctaggagatccagttacctcaggtttattccagttaaaaacttatctccgaatgattgagatggatgatttattcttacctttaattgcgaagaaccctgggaactgcactgccattgactttgtgctaaatcctagcggattaaatgttcctgggtcgcaagacttaacttcatttctgcgccagattgtacgtaggactatcaccctaagtgcgaaaaacaaacttattaataccttatttcatgcatcagctgacttcgaagacgaaatggtttgtaagtggctcttatcatcaactcctgttatgagtcgtttcgcagccgatatattttcacgcacgccgagcgggaagcgattgcaaattctaggatacttggaaggaacacgcacattattagcctctaagatcatcaacaataatacagagacgccggttttggacagactgaggaagataacattgcaaaggtggagtctatggtttagttatcttgatcattgtgataatatcctggcggaggctttaacccaaataacttgcacagttgatttagcacagatcctgagggaatattcatgggcacatattttagaggggagacctcttattggagccacactcccatgtatgattgagcaattcaaagtggtttggctgaaaccctacgaacaatgtccgcagtgttcaaatgccaagcaacctggtgggaaaccattcgtgtcagtagcagtcaagaaacatattgttagtgcatggccaaatgcatcccgaataagctggactatcggggatggaatcccatacattggatcaaggacagaagataagatagggcaacctgctattaaaccaaaatgtccttccgcagccttaagagaggccattgaattggcgtcccgtttaacatgggtaactcaaggcagttcgaacagtgacttgctaataaaaccatttttggaagcacgagtaaatttaagtgttcaagaaatacttcaaatgaccccttcacattactcgggaaatattgttcataggtacaacgatcaatacagtcctcattctttcatggccaatcgtatgagtaactcagcaacgcgattgattgtttctacaaacactttaggtgagttttcaggaggtggccaatcggcacgcgacagcaatattattttccagaatgttataaattatgcagttgcactgttcgatattaaatttagaaacactgaggctacagatatccagtataatcgtgctcaccttcatctaactaagtgttgcacccgggaggtaccagctcagtacttaacatacacatctacattggatttagatttaacaagataccgagaaaatgaattgatttatgacaataatcctctaaaaggaggactcaattgcaatatctcatttgataacccatttttccaaggcaaacagctgaacattatagaagatgaccttattcgactgcctcacttatctggatgggagctagctaagaccatcatgcaatcaattatttcagatagcaataattcgtctacagacccaattagcagtggagaaacaagatcattcactacccatttcttaacttatcccaaaataggacttctgtacagttttggggcctttgtaagttattatcttggcaatacaattcttcggactaagaaattaacacttgacaattttttatattacttaactacccaaattcataatctaccacatcgctcattgcgaatacttaagccaacattcaaacatgcaagcgttatgtcacgattaatgagtattgatccccatttttctatttacataggcggtgctgcaggtgacagaggactctcagatgcggccaggttatttttgagaacgtccatttcatcttttcttacatttgtaaaggaatggataattaatcgcggaacaattgtccctttatggatagtatatccattagagggtcaaaatccaacacctgttaataatttcctccatcagatcgtagaactgctggtgcatgattcatcaagacaccaggcttttaaaactaccataaatgatcatgtacatcctcacgacaatcttgtttacacatgtaagagtacagccagcaatttcttccatgcgtcattggcgtactggaggagcaggcacagaaacagcaaccgaaaagacttgacaagaaactcttcaactggatcaagcacaaacaacagtgatggtcatattaagagaagtcaagaacaaaccaccagagatccacatgatggcactgaacggagtctagtcctgcaaatgagccatgaaataaaaagaacgacaattccacaagagaacacgcaccagggtccgtcgttccagtcatttctaagtgactctgcttgcggtacagcaaacccaaaactaaatttcgatagatcgagacacaatgtgaaatctcaggatcataactcagcatccaagagggaaggtcatcaaataatctcacatcgtctagtcctacctttctttacattatctcaagggacacgccaattaacgtcatccaatgagtcacaaacccaagatgagatatcaaagtacttacggcaattgagatccgtcattgataccacagtttattgtaggtttaccggtatagtctcgtccatgcattacaaacttgatgaggtcctttgggaaatagagaattttaagtcggctgtgacgctggcagagggagaaggtgctggtgccttactattgattcagaaataccaagttaagaccttattcttcaacacgctagctactgagtccagtatagagtcagaaatagtatcaggaatgactactcctaggatgcttctacctgttatgtcaaaattccataatgaccaaattgagattattcttaacaactcagcaagccaaataacagacataacaaatcctacttggtttaaagaccaaagagcaaggctacctaggcaagtcgaggttataaccatggatgcagagacgacagagaatataaacagatcgaaattgtacgaagctgtacataaattgatcttacaccatgttgatcccagcgtattgaaagcagtggtccttaaagtctttctaagtgataccgagggtatgttatggctaaatgataatctagccccgttttttgccactgggtatttaattaagccaataacgtcaagtgccaggtctagtgagtggtatctttgtctgacgaacttcttatcaactacacgtaagatgccacaccaaaaccatctcagttgtaagcaggtaatacttacggcattgcaactgcaaattcaacggagcccatactggctaagtcatttaactcagtatgctgactgcgatttacatttaagctatatccgccttggttttccatcattagagaaagtactataccacaggtataaccttgtcgattcaaaaagaggtccactagtctctgtcactcagcacttagcacatcttagggcagagattcgagaattgaccaatgattataatcaacagcgacaaagtcggactcaaacatatcactttattcgtactgcaaaaggacgaatcacaaaactagtcaatgattatttaaaattctttcttattgtacaagcattaaaacataatgggacatggcaagctgagtttaagaaattaccagagttgattagtgtgtgcaataggttctatcatattagagattgtaattgtgaagaacgtttcttagttcaaaccttatatttacatagaatgcaggattctgaagttaagcttatcgaaaggctgacagggcttctgagtttatttccagatggtctctacaggttcgattgaataaccgtgcatagtattttgatacttgtaaaggttggttatcaacatacagattataaaaaactcataaattgctctcatacatcatcttgatctgatttcaataaataactatttagataacgaaaggagtccttacattatacactatatttggcctctctccctgcgtgataatcaaaaaattcacaatacagcatgtgtgacatattactgctgcaatgagtctaacgcaacataataaactccgcactctttataattaagctttaacgataggtctgggctcatattgttattgatatagtaatgttgtatcaatatcttgccagatggaatagtgctttggttgataacacgacttcttaaaacaaaactgatctttaagattaagttttttataattgtcattgctttaatttgtcgatttaaaaatggtgatagccttaatctttgtgtaaaataagagattaggtgtaataactttaacatttttgtctagtaagctactattccattcagaatgataaaattaaaagaaaagacatgactgtaaaatcagaaataccttctttacaatatagcagactagataataatcttcgtgttaatgataattaaggcattgaccacgctcatcagaaggctcactagaataaacgttgcaaaaaggatccctggaaaaatggtcgcacacaaaaatttaaaaataaatctatttcttcttttttgtgtgtcc

SEQ ID NO: 93 - DNA, 18958 bp, Ebola virus
cggacacacaaaaagaaagaagaatttttaggatcttttgtgtgcgaataactatgaggaagattaataattttcctctcattgaaatttatatcggaatttaaattgaaattgttactgtaatcatacctggtttgtttcagagccatatcaccaagatagagaacaacctaggtctccggagggggcaagggcatcagtgtgctcagttgaaaatcccttgtcaacatctaggccttatcacatcacaagttccgccttaaactctgcagggtgatccaacaaccttaatagcaacattattgttaaaggacagcattagttcacagtcaaacaagcaagattgagaattaactttgattttgaacctgaacacccagaggactggagactcaacaaccctaaagcctggggtaaaacattagaaatagtttaaagacaaattgctcggaatcacaaaattccgagtatggattctcgtcctcagaaagtctggatgacgccgagtctcactgaatctgacatggattaccacaagatcttgacagcaggtctgtccgttcaacaggggattgttcggcaaagagtcatcccagtgtatcaagtaaacaatcttgaggaaatttgccaacttatcatacaggcctttgaagctggtgttgattttcaagagagtgcggacagtttccttctcatgctttgtcttcatcatgcgtaccaaggagattacaaacttttcttggaaagtggcgcagtcaagtatttggaagggcacgggttccgttttgaagtcaagaagtgtgatggagtgaagcgccttgaggaattgctgccagcagtatctagtgggagaaacattaagagaacacttgctgccatgccggaagaggagacgactgaagctaatgccggtcagttcctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttttgatcaaatttcttctaatacaccaagggatgcacatggttgccggacatgatgccaacgatgctgtgatttcaaattcagtggctcaagctcgtttttcaggtctattgattgtcaaaacagtacttgatcatatcctacaaaagacagaacgaggagttcgtctccatcctcttgcaaggaccgccaaggtaaaaaatgaggtgaactccttcaaggctgcactcagctccctggccaagcatggagagtatgctcctttcgcccgacttttgaacctttctggagtaaataatcttgagcatggtcttttccctcaactgtcggcaattgcactcggagtcgccacagcccacgggagcaccctcgcaggagtaaatgttggagaacagtatcaacagctcagagaggcagccactgaggctgagaagcaactccaacaatatgcggagtctcgtgaacttgaccatcttggacttgatgatcaggaaaagaaaattcttatgaacttccatcagaaaaagaacgaaatcagcttccagcaaacaaacgcgatggtaactctaagaaaagagcgcctggccaagctgacagaagctatcactgctgcatcactgcccaaaacaagtggacattacgatgatgatgacgacattccctttccaggacccatcaatgatgacgacaatcctggccatcaagatgatgatccgactgactcacaggatacgaccattcccgatgtggtagttgaccccgatgatggaggctacggcgaataccaaagttactcggaaaacggcatgagtgcaccagatgacttggtcctattcgatctagacgaggacgacgaggacaccaagccagtgcctaacagatcgaccaagggtggacaacagaaaaacagtcaaaagggccagcatacagagggcagacagacacaatccacgccaactcaaaacgtcacaggccctcgcagaacaatccaccatgccagtgctccactcacggacaatgacagaagaaacgaaccctccggctcaaccagccctcgcatgctgaccccaatcaacgaagaggcagacccactggacgatgccgacgacgagacgtctagccttccgcccttagagtcagatgatgaagaacaggacagggacggaacttctaaccgcacacccactgtctccccaccggctcccgtatacagagatcactccgaaaagaaagaactcccgcaagatgaacaacaagatcaggaccacattcaagaggccaggaaccaagacagtgacaacacccagccagaacattcttttgaggagatgtatcgccacattctaagatcacaggggccatttgatgccgttttgtattatcatatgatgaaggatgagcctgtagttttcagtaccagtgatggtaaagagtacacgtatccggactcccttgaagaggaatatccaccatggctcactgaaaaagaggccatgaatgatgagaatagatttgttacactggatggtcaacaattttattggccagtaatgaatcacaggaataaattcatggcaatcctgcaacatcatcagtgaatgagcatgtaataatgggatgatttaatcgacaaatagctaacattaaatagtcaaggaacgcaaacaggaagaatttttgatgtctaaggtgtgaattattatcacaataaaagtgattcttagttttgaatttaaagctagcttattattactagccgtttttcaaagttcaatttgagtcttaatgcaaataagcgttaagccacagttatagccataatggtaactcaatatcttagccagcgatttatctaaattaaattacattatgcttttataacttacctactagcctgcccaacatttacacgatcgttttataattaagaaaaaactaatgatgaagattaaaaccttcatcatccttacgtcaattgaattctctagcactagaagcttattgtcttcaatgtaaaagaaaagctggcctaacaagatgacaactagaacaaagggcaggggccatactgtggccacgactcaaaacgacagaatgccaggccctgagctttcgggctggatctctgagcagctaatgaccggaaggattcctgtaaacgacatcttctgtgatattgagaacaatccaggattatgctacgcatcccaaatgcaacaaacgaagccaaacccgaagatgcgcaacagtcaaacccaaacggacccaatttgcaatcatagttttgaggaggtagtacaaacattggcttcattggctactgttgtgcaacaacaaaccatcgcatcagaatcattagaacaacgcattacgagtcttgagaatggtctaaagccagtttatgatatggcaaaaacaatctcctcattgaacagggtttgtgctgagatggttgcaaaatatgatcttctggtgatgacaaccggtcgggcaacagcaaccgctgcggcaactgaggcttattgggctgaacatggtcaaccaccacctggaccatcactttatgaagaaagtgcgattcggggtaagattgaatctagagatgagactgtccctcaaagtgttagggaggcattcaacaatctagacagtaccacttcactaactgaggaaaattttgggaaacctgacatttcggcaaaggatttgagaaacattatgtatgatcacttgcctggttttggaactgctttccaccaattagtacaagtgatttgtaaattgggaaaagatagcaattcattggacattattcatgctgagttccaggccagcctggctgaaggagactcccctcaatgtgccctaattcaaattacaaaaagagttccaatcttccaagatgctgctccacctgtcatccacatccgctctcgaggtgacattccccgagcttgccagaagagcttgcgtccagtcccaccatcacccaagattgatcgaggttgggtatgtgtttttcagcttcaagatggtaaaacacttggactcaaaatttgagccaatctcttttccctccgaaagaggcaactaatagcagaggcttcaactgctgaactatagggtatgttacattaatgatacacttgtgagtatcagccctagataatataagtcaattaaacaaccaagataaaattgttcatatcccgctagcagctttaaagataaatgtaataggagctatacctctgacagtattataattaattgttattaagtaacccaaaccaaaaatgatgaagattaagaaaaacctacctcgactgagagagtgttttttcattaaccttcatcttgtaaacgttgagcaaaattgttaaaaatatgaggcgggttatattgcctactgctcctcctgaatatatggaggccatataccctgccaggtcaaattcaacaattgctaggggtggcaacagcaatacaggcttcctgacaccggagtcagtcaatggagacactccatcgaatccactcaggccaattgctgatgacaccatcgaccatgccagccacacaccaggcagtgtgtcatcagcattcatcctcgaagctatggtgaatgtcatatcgggccccaaagtgctaatgaagcaaattccaatttggcttcctctaggtgtcgctgatcaaaagacctacagctttgactcaactacggccgccatcatgcttgcttcatatactatcacccatttcggcaaggcaaccaatccgcttgtcagagtcaatcggctgggtcctggaatcccggatcaccccctcaggctcctgcgaattggaaaccaggctttcctccaggagttcgttcttccaccagtccaactaccccagtatttcacctttgatttgacagcactcaaactgatcactcaaccactgcctgctgcaacatggaccgatgacactccaactggatcaaatggagcgttgcgtccaggaatttcatttcatccaaaacttcgccccattcttttacccaacaaaagtgggaagaaggggaacagtgccgatctaacatctccggagaaaatccaagcaataatgacttcactccaggactttaagatcgttccaattgatccaaccaaaaatatcatgggtatcgaagtgccagaaactctggtccacaagctgaccggtaagaaggtgacttccaaaaatggacaaccaatcatccctgttcttttgccaaagtacattgggttggacccggtggctccaggagacctcaccatggtaatcacacaggattgtgacacgtgtcattctcctgcaagtcttccagctgtggttgagaagtaattgcaataattgactcagatccagttttacagaatcttctcagggatagtgataacatctttttaataatccgtctactagaagagatacttctaattgatcaatatactaaaggtgctttacaccattgtctcttttctctcctaaatgtagagcttaacaaaagactcataatatacctgtttttaaaagattgattgatgaaagatcatgactaataacattacaaacaatcctactataatcaatacggtgattcaaatgtcaatctttctcattgcacatactctttgtccttatcctcaaattgcctacatgcttacatctgaggacagccagtgtgacttggattggagatgtggaggaaaaatcggggcccatttctaagttgttcacaatctaagtacagacattgctcttctaattaagaaaaaatcggcgatgaagattaagccgacagtgagcgtaatcttcatctctcttagattatttgtcttccagagtaggggtcatcaggtccttttcaattggataaccaaaataagcttcactagaaggatattgtgaggcgacaacacaatgggtgttacaggaatattgcagttacctcgtgatcgattcaagaggacatcattctttctttgggtaattatccttttccaaagaacattttccatcccgcttggagttatccacaatagtacattacaggttagtgatgtcgacaaactagtttgtcgtgacaaactgtcatccacaaatcaattgagatcagttggactgaatctcgaggggaatggagtggcaactgacgtgccatctgtgactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccatgtgccggagactttgccttccacaaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtcgagtggctattattctaccacaattagatatcaggctaccggttttggaactaatgagacagagtacttgttcgaggttgacaatttgacctacgtccaacttgaatcaagattcacaccacagtttctgctccagctgaatgagacaatatatgcaagtgggaagaggagcaacaccacgggaaaactaatttggaaggtcaaccccgaaattgatacaacaatcggggagtgggccttctgggaaactaaaaaaacctcactagaaaaattcgcagtgaagagttgtctttcacagctgtatcaaacggacccaaaaacatcagtggtcagagtccggcgcgaacttcttccgacccagagaccaacacaacaaatgaagaccacaaaatcatggcttcagaaaattcctctgcaatggttcaagtgcacagtcaaggaaggaaagctgcagtgtcgcatctgacaacccttgccacaatctccacgagtcctcaacctcccacaaccaaaacaggtccggacaacagcacccataatacacccgtgtataaacttgacatctctgaggcaactcaagttggacaacatcaccgtagagcagacaacgacagcacagcctccgacactccccccgccacgaccgcagccggacccttaaaagcagagaacaccaacacgagtaagagcgctgactccctggacctcgccaccacgacaagcccccaaaactacagcgagactgctggcaacaacaacactcatcaccaagataccggagaagagagtgccagcagcgggaagctaggcttaattaccaatactattgctggagtagcaggactgatcacaggcgggagaaggactcgaagagaagtaattgtcaatgctcaacccaaatgcaaccccaatttacattactggactactcaggatgaaggtgctgcaatcggattggcctggataccatatttcgggccagcagccgaaggaatttacacagaggggctaatgcacaaccaagatggtttaatctgtgggttgaggcagctggccaacgaaacgactcaagctctccaactgttcctgagagccacaactgagctgcgaaccttttcaatcctcaaccgtaaggcaattgacttcctgctgcagcgatggggtggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggtgttataattgcagttatcgctttattctgtatatgcaaatttgtcttttagtctttcttcagattgtttcacggcaaaactcaacctcaaatcaatgaaactaggatttaattatatgaatcacttgaatctaagattacttgacaaatgataacataatacactggagcttcaaacatagccaatgtgattctaactcctttaaactcacagttaatcataaacaaggtttgacatcaatctagctatatctttaagaatgataaacttgatgaagattaagaaaaaggtaatctttcgattatctttagtcttcatccttgattctacaatcatgacagttgtctttaatgaaaaaggaaaaaagcctttttattaagttgtaataatcagatctgcaaaccggtagaatttagttgtaacctaacacacacaaagcattggtaaaaaagtcaatagaaatttaaacagtgagtgcagacaactcttaaatggaagcttcatatgagagaggacgcccccgagctgccagacagcattcaagggatggacacgaccaccatgttcgagcacgatcatcatccagagagaattatcgaggtgagtaccgtcaatcaaggagcgcctcacaagtgcgcgttcctactgtatttcataagaagagagttgaaccattaacagttcctccagcacctaaagacatatgtccgaccttgaaaaaaggatttttgtgtgacagtagtttttgcaaaaaagaccaccagttagaaagtttaactgatagggaattactcctactaatcgcccgtaagacttgtggatcagtagaacaacaattaaatataactgcacccaaggactcgcgcttagcaaatccaacggctgatgatttccagcaagaggaaggtcccaaaattaccttgttgacactgatcaagacggcagaacactgggcgagacaagacatccgaaccatagaggattccaaattaagggcattgttaactctatgtgctgtgatgacgaggaaattctcaaaatcccagctgagtcttttgtgtgagacacacctaaggcgcgaagggcttgggcaagatcaggcagaacccgttctcgaagtatatcaacgattacacagtgataaaggaggcagttttgaagctgcactatggcaacaatgggaccgacaatccctaattatgtttatcactgcattcttgaatatcgctctccagttaccgtgtgaaagttctgctgtcgttgtttcagggttaagaacattggttcctcaatcagataatgaggaagcttcaaccaacccggggacatgctcatggtctgatgagggtaccccttaataaggctgactaaaacactatataaccttctacttgatcacaatactccgtatacctatcatcatatatttaatcaagacgatatcctttaaaacttattcagtactataatcactctcatttcaaattgataagatatgcataattgccttaatatataaagaggtatgatataacccaaacattgaccaaagaaaatcataatctcgtatcgctcgcaatataacctgccaagcatacctcttgcacaaagtgattcttgtacacaaataatgtttgactctacaggaggtagcaacgatccatctcatcaaaaaataagtattttatgatttactaatgatctcttaaaatattaagaaaaactgacggaacataaattctttctgcttcaagttgtggaggaggtctatggtattcgctattgttatattacaatcaataacaagcttgtaaaaatattgttcttgtttcaggaggtatattgtgaccggaaaagctaaactaatgatgaagattaatgcggaggtctgatgagaataaaccttattattcagattaggccccaagaggcattcttcatctccttttagcaaaatactatttcaggatagtccagctagtgacacgtcttttagctgtataccagttgcccctgagatacgccacaaaagtgtctctgagctaaagtggtctgtacacatctcatacattgtattaggggcaataatatctaattgaacttagccatttaaaatttagtgcataaatctgggctaactccaccaggtcaactccattggctgaaaagaagcccacctacaacgaacattactttgagcgccctcacaattaaaaaataagagcgtcgttcctacaatcgagcgcaaggttacaaggttgaactgagagtgtctagacaacaaaatatcgatactccagacaccaagcaagacctgagaaaaaaccatggccaaagctacgggacgatacaatctaatatcgcccaaaaaggacctggagaaaggggttgtcttaagcgacctctgcaacttcttagttagtcaaactattcaagggtggaaagtttattgggctggtattgagtttgatgtgactcacaaaggaatggccctattgcatagactgaaaactaatgactttgcccctgcatggtcaatgacaaggaacctatttccccatttatttcaaaatccgaattccactattgaatcaccgctgtgggcactgagagtcatccttgcagcagggatacaggaccagttaattgaccagtctttgattgaacccttagcaggagcccttggtctgatctctgattggctgctaacaaccaacactaaccatttcaacatgcgaacacaacgtgtcaaggaacaattgagcctaaaaatgctgtcgttgattcgatccaatattctcaagtttattaacaaattggatgctctacatgtcgtgaactacaatggattattgagcagtattgaaattggaactcaaaatcatacaatcatcataactcgaactaacatgggttttctggtggagctccaagaacccgacaaatcggcaatgaaccgcaagaagcctgggccggcgaaattttccctccttcatgagtccacactgaaagcatttacacaagggtcctcgacacgaatgcaaagtttaattcttgaattcaatagctctcttgctatctaactaagatggaatacttcatattgggctaactcatatatgctgactcaatagttaacttgacatctctgccttcataatcagatatataagcataataaataaatactcatatttcttgataatttgtttaaccacagataaatcctcactgtaagccagcttccaagttgacacccttacaaaaaccaggactcagaatccctcaaataagagattccaagacaacatcatagaattgctttattatattaataagcattttatcactagaaatccaatatacgaaatggttaattgtaactaaacccgcaggtcatgtgtgttaggtttcacaaattatatatattactaactccatactcgtaactaacattagataagtaggttaagaaaaaagcttgaggaagattaagaaaaactgcttattgggtctttccgtgttttagatgaagcagttgacattcttcctcttgatattaaatggctacacaacatacccaatacccagacgccaggctatcatcaccaattgtattggaccaatgtgaccttgtcactagagcttgcgggttgtattcatcatactcccttaatccgcaactacgcaactgtaaactcccgaaacatatataccgtttaaaatatgatgtaactgttaccaagttcttaagtgatgtaccagtggcgacattgcccatagatttcatagtcccaattcttctcaaggcactatcaggcaatgggttctgtcctgttgagccgcggtgccaacagttcttagatgaaattattaagtacacaatgcaagatgctctcttcctgaaatattatctcaaaaatgtgggtgctcaagaagactgtgttgatgaccactttcaagaaaaaatcttatcttcaattcagggcaatgaatttttacatcaaatgtttttctggtatgacctggctattttaactcgaaggggtagattaaatcgaggaaactctagatcaacgtggtttgttcatgatgatttaatagacatcttaggctatggggactatgttttttggaagatcccaatttcactgttaccactgaacacacaaggaatcccccatgctgctatggattggtatcagacatcagtattcaaagaagcggttcaagggcatacacacattgtttctgtttctactgccgatgtcttgataatgtgcaaagatttaattacatgtcgattcaacacaactctaatctcaaaaatagcagaggttgaggacccagtttgctctgattatcccaattttaagattgtgtctatgctttaccagagcggagattacttactctccatattagggtctgatgggtataaaatcattaagtttctcgaaccattgtgcttggctaaaattcaattgtgctcaaagtacaccgagaggaagggccgattcttaacacaaatgcatttagctgtaaatcacaccctggaagaaattacagaaatacgtgcactaaagccttcacaggctcacaagatccgtgaattccatagaacattgataaggctggagatgacgccacaacaactttgtgagctattttccatacaaaaacactgggggcatcctgtgctacatagtgaaacagcaatccaaaaagttaaaaaacatgctacggtgctaaaagcattacgccctatcgtgattttcgagacatattgtgtttttaaatatagcattgcaaaacattattttgatagtcaaggatcttggtacagtgttacctcagatagaaatctaacaccaggtcttaattcttatatcaaaagaaatcaattccctccgttgccaatgattaaagaactgctatgggaattttaccaccttgaccatcctccacttttctcaaccaaaattattagtgacttaagtatttttataaaagacagagctactgcagtagaaaggacatgctgggatgcagtattcgagcctaatgttctgggatataatccacctcacaaattcagtaccaaacgtgtaccggaacaatttttagagcaagaaaacttttctattgagaatgttctttcctacgcgcaaaaactcgagtatctactaccacaatatcggaatttttctttctcattgaaagagaaagagttgaatgtaggtagaactttcggaaaattgccttacccgactcgcaatgttcaaacactttgtgaagctctgttagctgatggtcttgctaaagcatttcctagcaatatgatggtagttacggaacgtgaacaaaaagaaagcttattgcatcaagcatcatggcaccacacaagtgatgatttcggtgagcatgccacagttagagggagtagctttgtaactgatttagagaaatacaatcttgcatttaggtatgagtttacagcaccttttatagaatattgcaaccgttgctatggtgttaagaatgtttttaattggatgcattatacaatcccacagtgttatatgcatgtcagtgattattataatccaccgcataacctcacactggaaaatcgaaacaacccccctgaagggcctagttcatacaggggtcatatgggagggattgaaggactgcaacaaaaactctggacaagtatttcatgtgctcaaatttctttagttgaaattaagactggttttaagttgcgctcagctgtgatgggtgacaatcagtgcattaccgttttatcagtcttccccttagagactgatgcaggcgagcaggaacagagcgccgaggacaatgcagcgagggtggccgccagcctagcaaaagttacaagtgcctgtggaatctttttaaaacctgatgaaacatttgtacattcaggttttatctattttggaaaaaaacaatatttgaatggggtccaattgcctcagtcccttaaaacggctacaagaatggcaccattgtctgatgcaatttttgatgatcttcaagggaccctggctagtataggtactgcttttgagcgatccatctctgagacacgacatatctttccttgcagaataaccgcagctttccatacgttcttttcggtgagaatcttgcaatatcatcacctcggatttaataaaggttttgaccttggacagttaacactcggcaaacctctggatttcggaacaatatcattggcactagcggtaccgcaggtgcttggagggttatccttcttgaatcctgagaaatgtttctaccggaatctaggagatccagttacctcaggtttattccagttaaaaacttatctccgaatgattgagatggatgatttattcttacctttaattgcgaagaaccctgggaactgcactgccattgactttgtgctaaatcctagcggattaaatgttcctgggtcgcaagacttaacttcatttctgcgccagattgtacgtaggactatcaccctaagtgcgaaaaacaaacttattaataccttatttcatgcatcagctgacttcgaagacgaaatggtttgtaagtggctcttatcatcaactcctgttatgagtcgtttcgcagccgatatattttcacgcacgccgagcgggaagcgattgcaaattctaggatacttggaaggaacacgcacattattagcctctaagatcatcaacaataatacagagacgccggttttggacagactgaggaagataacattgcaaaggtggagtctatggtttagttatcttgatcattgtgataatatcctggcggaggctttaacccaaataacttgcacagttgatttagcacagatcctgagggaatattcatgggcacatattttagaggggagacctcttattggagccacactcccatgtatgattgagcaattcaaagtggtttggctgaaaccctacgaacaatgtccgcagtgttcaaatgccaagcaacctggtgggaaaccattcgtgtcagtagcagtcaagaaacatattgttagtgcatggccaaatgcatcccgaataagctggactatcggggatggaatcccatacattggatcaaggacagaagataagatagggcaacctgctattaaaccaaaatgtccttccgcagccttaagagaggccattgaattggcgtcccgtttaacatgggtaactcaaggcagttcgaacagtgacttgctaataaaaccatttttggaagcacgagtaaatttaagtgttcaagaaatacttcaaatgaccccttcacattactcgggaaatattgttcataggtacaacgatcaatacagtcctcattctttcatggccaatcgtatgagtaactcagcaacgcgattgattgtttctacaaacactttaggtgagttttcaggaggtggccaatcggcacgcgacagcaatattattttccagaatgttataaattatgcagttgcactgttcgatattaaatttagaaacactgaggctacagatatccagtataatcgtgctcaccttcatctaactaagtgttgcacccgggaggtaccagctcagtacttaacatacacatctacattggatttagatttaacaagataccgagaaaatgaattgatttatgacaataatcctctaaaaggaggactcaattgcaatatctcatttgataacccatttttccaaggcaaacagctgaacattatagaagatgaccttattcgactgcctcacttatctggatgggagctagctaagaccatcatgcaatcaattatttcagatagcaataattcgtctacagacccaattagcagtggagaaacaagatcattcactacccatttcttaacttatcccaaaataggacttctgtacagttttggggcctttgtaagttattatcttggcaatacaattcttcggactaagaaattaacacttgacaattttttatattacttaactacccaaattcataatctaccacatcgctcattgcgaatacttaagccaacattcaaacatgcaagcgttatgtcacgattaatgagtattgatccccatttttctatttacataggcggtgctgcaggtgacagaggactctcagatgcggccaggttatttttgagaacgtccatttcatcttttcttacatttgtaaaggaatggataattaatcgcggaacaattgtccctttatggatagtatatccattagagggtcaaaatccaacacctgttaataatttcctccatcagatcgtagaactgctggtgcatgattcatcaagacaccaggcttttaaaactaccataaatgatcatgtacatcctcacgacaatcttgtttacacatgtaagagtacagccagcaatttcttccatgcgtcattggcgtactggaggagcaggcacagaaacagcaaccgaaaagacttgacaagaaactcttcaactggatcaagcacaaacaacagtgatggtcatattaagagaagtcaagaacaaaccaccagagatccacatgatggcactgaacggagtctagtcctgcaaatgagccatgaaataaaaagaacgacaattccacaagagaacacgcaccagggtccgtcgttccagtcatttctaagtgactctgcttgcggtacagcaaacccaaaactaaatttcgatagatcgagacacaatgtgaaatctcaggatcataactcagcatccaagagggaaggtcatcaaataatctcacatcgtctagtcctacctttctttacattatctcaagggacacgccaattaacgtcatccaatgagtcacaaacccaagatgagatatcaaagtacttacggcaattgagatccgtcattgataccacagtttattgtaggtttaccggtatagtctcgtccatgcattacaaacttgatgaggtcctttgggaaatagagaattttaagtcggctgtgacgctggcagagggagaaggtgctggtgccttactattgattcagaaataccaagttaagaccttattcttcaacacgctagctactgagtccagtatagagtcagaaatagtatcaggaatgactactcctaggatgcttctacctgttatgtcaaaattccataatgaccaaattgagattattcttaacaactcagcaagccaaataacagacataacaaatcctacttggtttaaagaccaaagagcaaggctacctaggcaagtcgaggttataaccatggatgcagagacgacagagaatataaacagatcgaaattgtacgaagctgtacataaattgatcttacaccatgttgatcccagcgtattgaaagcagtggtccttaaagtctttctaagtgataccgagggtatgttatggctaaatgataatctagccccgttttttgccactgggtatttaattaagccaataacgtcaagtgccaggtctagtgagtggtatctttgtctgacgaacttcttatcaactacacgtaagatgccacaccaaaaccatctcagttgtaagcaggtaatacttacggcattgcaactgcaaattcaacggagcccatactggctaagtcatttaactcagtatgctgactgcgatttacatttaagctatatccgccttggttttccatcattagagaaagtactataccacaggtataaccttgtcgattcaaaaagaggtccactagtctctgtcactcagcacttagcacatcttagggcagagattcgagaattgaccaatgattataatcaacagcgacaaagtcggactcaaacatatcactttattcgtactgcaaaaggacgaatcacaaaactagtcaatgattatttaaaattctttcttattgtacaagcattaaaacataatgggacatggcaagctgagtttaagaaattaccagagttgattagtgtgtgcaataggttctatcatattagagattgtaattgtgaagaacgtttcttagttcaaaccttatatttacatagaatgcaggattctgaagttaagcttatcgaaaggctgacagggcttctgagtttatttccagatggtctctacaggttcgattgaataaccgtgcatagtattttgatacttgtaaaggttggttatcaacatacagattataaaaaactcataaattgctctcatacatcatcttgatctgatttcaataaataactatttagataacgaaaggagtccttacattatacactatatttggcctctctccctgcgtgataatcaaaaaattcacaatacagcatgtgtgacatattactgctgcaatgagtctaacgcaacataataaactccgcactctttataattcagctttaacgataggtctgggctcatattgttattgatatagtaatgttgtatcaatatcttgccagatggaatagtgctttggttgataacacgacttcttaaaacaaaactgatctttaagattaagttttttataattgtcattgctttaatttgtcgatttaaaaatggtgatagccttaatctttgtgtaaaataagagattaggtgtaataactttaacatttttgtctagtaagctactattccattcagaatgataaaattaaaagaaaagacatgactgtaaaatcagaaataccttctttacaatatagcagactagataataatcttcgtgttaatgataattaaggcattgaccacgctcatcagaaggctcactagaataaacgttgcaaaaaggatccctggaaaaatggtcgcacacaaaaatttaaaaataaatctatttcttcttttttgtgtgtcc

SEQ ID NO: 94 - DNA, 399 bp, Mus musculus; identical to SEQ ID NO: 5

SEQ ID NO: 95 - DNA, 399 bp, Artificial Sequence
  A synthetic oligonucleotide
gggtccgaggtgaagctgcaggagtctggacctggactggtggcaccatctcaaagcctgagcatcacttgtaccgttagtggcttctcattttcccgatacaccgtccattgggtcagacagcctcccggtaaagggctggaatggctgggcatgatatggggtggaggatccactgattacaatagcgcactgaaaagccgcctgtctatttccaaggacaattccaaaagtcaggtgtttctcgaaatgaacagcctgcagacagatgacacagcaatgtattattgcgttcggagtggaaactggaacgcgatggactactggggccagggaacctcagtgacagtttccagcgctaagactacgcccccaagcgtgtacgggggcggagggtct

SEQ ID NO: 96 - DNA, 399 bp, Artificial Sequence
  A synthetic oligonucleotide
ggcagcgaggtcaaactgcaggaatccggcccaggccttgtcgccccctcccaatcactgagcatcacctgcacggttagtggcttctcctttagtagatatacggttcactgggtccgacagcccccgggaaagggactggagtggcttggtatgatttgggggggcggctccacagactacaactctgcactcaagagtagactgtcaatcagcaaggataacagcaagtcccaggtcttcttggagatgaactcccttcagacagacgatactgccatgtactactgcgtgagatccggaaactggaatgctatggattattggggacagggaaccagtgtgacagttagctctgcaaaaacaacccccccttccgtctatggcgggggaggtagc

SEQ ID NO: 97 - DNA, 399 bp, Artificial Sequence
  A synthetic oligonucleotide
ggcagcgaagtgaagctgcaagagagcgggccagggctggtcgcaccttcccagagtctctccattacctgcactgtctccgggttctccttctctcgatacactgtgcattgggtccgacagccacctggaaagggcctggagtggctcgggatgatttggggcggcggctccaccgattataatagcgccctcaaatccagactgagtatatctaaggataattctaagagtcaggtattcctcgagatgaattccctgcagacggacgacactgcaatgtactattgcgttagatctggcaattggaacgctatggactactggggacagggtacgtcagtgacggtctcaagcgccaagaccactccgcctagtgtctacggcgggggcggtagt